(19) United States
(12) Patent Application Publication
(10) Pub. No.: US 2022/0101948 A1
(43) Pub. Date: Mar. 31, 2022

(54) METHODS OF IDENTIFYING GENETIC VARIANTS

(71) Applicants: The University of Sydney, Sydney (AU); The Sydney Children's Hospitals Network (Randwick and Westmead), Westmead (AU)

(72) Inventors: Sandra Cooper, Naremburn (AU); Himanshu Joshi, Peakhurst (AU)

(73) Assignees: The University of Sydney, Sydney (AU); The Sydney Children's Hospitals Network (Randwick and Westmead), Westmead (AU)

(21) Appl. No.: 17/319,986

(22) Filed: May 13, 2021

Related U.S. Application Data
(63) Continuation of application No. PCT/AU2019/000141, filed on Nov. 15, 2019.

(30) Foreign Application Priority Data
Nov. 15, 2018 (AU) 2018904348

(51) Int. Cl. G16B 30/10; G16B 50/30; C12Q 1/6869

(52) U.S. Cl. CPC G16B 30/10; C12Q 1/6869; G16B 50/30

(57) ABSTRACT

The present invention relates to identification of an abnormal splice site. Provided are methods of identifying an abnormal splice site. Methods of classifying the risk of abnormal splicing of a splice site are also provided. Databases for use in the methods provided herein are also disclosed.

### RELATED APPLICATIONS

This application is a continuation of International Patent Application No. PCT/AU2019/000141, filed Nov. 15, 2019, entitled “Methods of Identifying Genetic Variants”. Foreign priority benefits are claimed under 35 U.S.C. § 119(a)-(d) or 35 U.S.C. § 365(b) of Australian Application No. 2018904348, filed Nov. 15, 2018. The contents of each of these applications are incorporated herein by reference in their entirety.

### FIELD OF THE INVENTION

The present invention relates to identification of an abnormal splice site. In particular, provided are methods of identifying an abnormal splice site. Methods of classifying the risk of abnormal splicing of a splice site are also provided. Databases for use in the methods provided herein are also disclosed.

### BACKGROUND OF THE INVENTION

Any discussion of the prior art throughout the specification should in no way be considered as an admission that such prior art is widely known or forms part of common general knowledge in the field.

Splicing of pre-mRNA in eukaryotes involves recognition of exons and introns. During splicing, the borders of introns are recognized, cleaved, and exons are then ligated together. A splicing event requires the assembly of splicing machinery in spliceosome complexes on consensus elements present in the splice site (e.g., the donor splice site, the branch site, the acceptor splice site). Genetic variants affecting a splice site (an abnormal splice site) disrupt splicing processes leading to aberrant splicing and causing diseases, including inherited diseases (genetic disorders) and cancer.

Many abnormal splice sites remain unclassified (variant of unknown significance (VUS)), meaning their clinical significance also remains unclassified. Thus, patients with, for example, an inherited disease (genetic disorder) may not receive a genetic diagnosis. An understanding of the genetic cause of a disease is important to guide clinical management and enable personalised and precision medicine. Accordingly, determining the clinical significance of an abnormal splice site may lead to a genetic diagnosis to direct the clinical care and application and development of therapies.

It is an object of the present invention to overcome or ameliorate at least one of the disadvantages of the prior art, or to provide a useful alternative.

### SUMMARY OF THE INVENTION

The inventors recognized that variants of splice sites, which are not present in any splice site of the human genome, have a high likelihood of exhibiting abnormal splicing (eg reducing splicing, non-splicing, exon skipping, or any splicing event associated with a pathogenic phenotype) and are referred to herein as abnormal splice sites. Thus, herein provided are methods of identifying an abnormal splice site based on a determination of the presence or absence of a sample splice site, or a portion thereof, in any splice site in a reference human genome. This determination may be referred to herein as Native Intron Frequency. Thereby a risk of abnormal splicing of a sample splice site may be determined. A sample splice site that is absent from the human genome has a high risk of abnormal splicing. A sample splice site that is infrequently used in the human genome may have a high risk of abnormal splicing. The inventors recognized that the relative shift in frequency of a sample splice site, as determined by a comparison of frequency of a sample splice site with the frequency of the originating splice site (the spice site correlating to the sample splice site in the human genome (referred to herein as a reference splice site sequence)), may be used to determine a risk of abnormal splicing. The relative shift in frequency may be compared to a reference dataset comprising variant splice sites (with their corresponding relative shift in frequency in comparison to a reference human genome) and their classification (abnormal splice site or benign variant splice site). Thereby, a risk of abnormal splicing of a sample splice site may be determined.

Other factors may be used in conjunction with the measure of frequency of a splice site in the human genome to determine a risk of abnormal splicing of a sample splice site. One additional factor, which may be referred to as a previous classification factor, considers whether the splice site, or a portion thereof, has previously been classified clinically as an abnormal splice site or a benign variant splice site. A previous classification factor may be determined by comparing a sample splice site to a reference dataset of splice sites with a known clinical classification (e.g., abnormal splice site or benign variant splice site). Another additional factor, which may be referred to as a similar splice site frequency shift factor or (similar NIF-shift factor), considers the clinical classification (e.g., abnormal splice site or benign variant splice site) of variant splice sites having similar relative shifts in Native Intron Frequency to a sample splice site.

It will be appreciated that in the method herein described identification of an abnormal splice site in a sample splice site from a subject may comprise or consist of a determination of a risk of abnormal splicing of the sample splice site. Thereby, a risk of abnormal splicing of a sample splice site may be considered as a risk that a sample splice site is an abnormal splice site.

In a first embodiment, provided is a method of identifying an abnormal splice site in a sample splice site from a subject, said method comprising:
(a) obtaining a first sample splice site sequence comprised in the sample splice site from the subject; and
(b) determining a Native Intron Frequency of the first sample splice site sequence (NIFᵥₐᵣ₋₁); wherein a NIFᵥₐᵣ₋₁ of 0 (zero) indicates that the sample splice site is abnormal.

In further embodiments related to the first embodiment, the sample splice site may be a donor splice site. In certain embodiments, the sample splice site sequence comprises 4 to 12 nucleotides of a donor splice site. In certain embodiments, the sample splice site sequence comprises 4, 5, 6, 7, 8, 9, 10, 11, or 12 consecutive nucleotides of a donor splice site. In certain embodiments, the sample splice site sequence comprises 4 to 15 nucleotides of a donor splice site. In certain embodiments, the sample splice site sequence comprises 4, 5, 6, 7, 8, 9, 10, 11, 12, 13, 14 or up to 15 consecutive nucleotides of a donor splice site. In certain embodiments, the sample splice site sequence comprises 30 or more nucleotides of a donor splice site. In certain embodiments, the sample splice site sequence comprises 30 or more consecutive nucleotides of a donor splice site. In certain embodiments, the sample splice site sequence comprises 9 consecutive nucleotides of a donor splice site. In certain embodiments, the splice site is a donor splice site, steps (a) and (b) are repeated with a second sample splice site sequence comprised in the same sample splice site, and NIFᵥₐᵣ₋₂ is determined, wherein a NIFᵥₐᵣ of 0 (zero) for any sample splice site sequence indicates that the sample splice site is abnormal. In certain embodiments, the sample splice site is a donor splice site, and steps (a) and (b) are repeated with up to five additional sample donor splice site sequences comprised in the same sample splice site, and NIFᵥₐᵣ₋₂, NIFᵥₐᵣ₋₃, NIFᵥₐᵣ₋₄, NIFᵥₐᵣ₋₅, up to NIFᵥₐᵣ₋₆ are determined and correspond to the NIFᵥₐᵣ for each of the second, third, fourth, fifth, and up to the sixth sample donor splice site sequence, respectively, wherein a NIFᵥₐᵣ of 0 (zero) for any sample donor splice site sequence indicates that the sample donor splice site is abnormal. In certain embodiments, the sample splice site is a donor splice site, and steps (a) and (b) are repeated with up to five additional sample donor splice site sequences, wherein each sample donor splice site sequence comprises 9 non-identical consecutive nucleotides of the same sample donor splice site, and wherein one or more of the sample donor splice site sequences may comprise overlapping consecutive nucleotides of the donor splice site. In a related embodiment comprising at least six sample splice site sequences from the same sample splice site, the sample splice site sequences correspond to at least nucleotide positions E⁻⁵ to D⁺⁴, E⁻⁴ to D⁺⁵, E⁻³ to D⁺⁶, E⁻² to D⁺⁷, E⁻¹ to D⁺⁸, and D⁺¹ to D⁺⁹ of a donor splice site. In a related embodiment comprising at least four sample splice site sequences from the same sample splice site, the sample splice site sequences correspond to at least nucleotide positions E⁻⁴ to D⁺⁵, E⁻³ to D⁺⁶, E⁻² to D⁺⁷ and E⁻¹ to D⁺⁸ of a donor splice site, wherein the nomenclature E⁻⁴ to E⁻¹ corresponds to the last four nucleotides of an exon and D⁺¹ to D⁺⁸ correspond the first eight nucleotides of the intron.

In further embodiments related to the first embodiment, the sample splice site is a donor splice site. In certain embodiments, the sample splice site sequence comprises 6 to 15 nucleotides of a donor splice site. In certain embodiments, the sample splice site sequence comprises 12 consecutive nucleotides of a donor splice site that is analysed as a collective of multiple, overlapping donor reference splice site sequences, wherein the median of NIFᵥₐᵣ₋₁, NIFᵥₐᵣ₋₂, NIFᵥₐᵣ₋₃, NIFᵥₐᵣ₋₄ and up to NIFᵥₐᵣ₋₆, corresponding to NIFᵥₐᵣ for each of the first, second, third, fourth and up to sixth sample donor splice site sequences is determined. In certain embodiments, the sample splice site is a donor splice site of 12 nucleotides divided into four sample splice site sequences comprised of 9 non-identical sequences of consecutive nucleotides corresponding to nucleotide positions E⁻⁴ to D⁺⁵, E⁻³ to D⁺⁶, E⁻² to D⁺⁷ and E⁻¹ to D⁺⁸ of a donor splice site. The median NIFᵥₐᵣ₋ₓ is calculated as median (NIFᵥₐᵣ₋₁; NIFᵥₐᵣ₋₂; NIFᵥₐᵣ₋₃; NIFᵥₐᵣ₋₄) wherein a median NIFᵥₐᵣ₋ₓ of 0 (zero) for any sample donor splice site sequence indicates that the sample donor splice site is abnormal.

In further embodiments related to the first embodiment, the sample splice site is a donor splice site. In certain embodiments, the sample splice site sequence comprises 6 to 15 nucleotides of a donor splice site. In certain embodiments, the sample splice site sequence comprises 12 consecutive nucleotides of a donor splice site that is analysed as a collective of multiple, overlapping donor reference splice site sequences, wherein the percentile for each of NIFᵥₐᵣ₋₁, NIFᵥₐᵣ₋₂, NIFᵥₐᵣ₋₃, NIFᵥₐᵣ₋₄ and up to NIFᵥₐᵣ₋₆, corresponding to NIFᵥₐᵣ for each of the first, second, third, fourth and up to sixth sample donor splice site sequences is determined. In certain embodiments, the sample splice site is a donor splice site of 12 nucleotides divided into four sample splice site sequences comprised of 9 non-identical sequences of consecutive nucleotides corresponding to nucleotide positions E⁻⁴ to D⁺⁵, E⁻³ to D⁺⁶, E⁻² to D⁺⁷ and E⁻¹ to D⁺⁸ of a donor splice site. The median percentile NIFᵥₐᵣ₋ₓ is calculated as median (NIFᵥₐᵣ₋₁ percentile; NIFᵥₐᵣ₋₂ percentile; percentile of NIFᵥₐᵣ₋₃ percentile; NIFᵥₐᵣ₋₄ percentile) wherein a median percentile NIFᵥₐᵣ₋ₓ of 0 (zero) for any sample donor splice site sequence indicates that the sample donor splice site is abnormal.

In further embodiments related to the first embodiment, the sample splice site sequence comprises 12 consecutive nucleotides of a donor splice site that is analysed as a collective of multiple, overlapping donor reference splice site sequences, wherein the median NIFᵥₐᵣ₋ₓ is converted to a percentile value. For example, a sample splice site with a median NIFᵥₐᵣ₋ₓ of 0 (zero) lies within the zeroth percentile of a frequency distribution of median NIF_{ref-x} among all donor splice sites in the reference human genome. A sample donor splice site with median NIFᵥₐᵣ₋ₓ in the zeroth percentile indicates that the sample donor splice site is abnormal

In related embodiments, the use of median NIFᵥₐᵣ₋ₓ described in Section [0012] may be substituted for mean NIFᵥₐᵣ₋ₓ calculated as mean (NIFᵥₐᵣ₋₁; NIFᵥₐᵣ₋₂; NIFᵥₐᵣ₋₃; NIFᵥₐᵣ₋₄) and a mean NIFᵥₐᵣ₋ₓ of 0 (zero) for any sample donor splice site sequence indicates that the sample donor splice site is abnormal.

In related embodiments, the use of median NIFᵥₐᵣ₋ₓ converted to a percentile value described in Section [0013] may be substituted for mean (percentile of NIFᵥₐᵣ₋₁; percentile of NIFᵥₐᵣ₋₂; percentile of NIFᵥₐᵣ₋₃; percentile of NIFᵥₐᵣ₋₄) wherein a median percentile NIFᵥₐᵣ₋ₓ of 0 (zero) for any sample donor splice site sequence indicates that the sample donor splice site is abnormal.

In a second embodiment, provided is a method of identifying an abnormal splice site in a sample splice site from a subject, said method comprising:
(a) obtaining a first sample splice site sequence comprised in the sample splice site from the subject;
(b) determining a measure of Native Intron Frequency of the first sample splice site sequence (NIFᵥₐᵣ₋₁);
(c) determining a Percentile (NIFᵥₐᵣ₋₁) of the first sample splice site sequence;
(d) determining a measure of Native Intron Frequency of a first reference splice site sequence (NIF_{ref-1}); wherein the first reference splice site sequence and the first sample splice site sequence each originate from the same corresponding region of a gene;
(e) determining a Percentile (NIF_{ref-1}) of the first reference splice site sequence; and
(f) determining a risk of abnormal splicing for the sample splice site by comparing Percentile (NIFᵥₐᵣ₋₁) with Percentile (NIF_{ref-1}) against a Clinical Splice Predictor (CSP) reference database.

In a further embodiment related to the second embodiment, provided is a method of identifying an abnormal splice site in a sample splice site from a subject, said method comprising:
(a) obtaining a first sample splice site sequence comprised in the sample splice site from the subject;
(b) determining a measure of Native Intron Frequency of the first sample splice site sequence (NIFᵥₐᵣ₋₁);
(c) determining a measure of Native Intron Frequency of a first reference splice site sequence (NIF_{ref-1}); wherein the first reference splice site sequence and the first sample splice site sequence each originate from the same corresponding region of a gene; and
(d) determining a risk of abnormal splicing for the sample splice site by comparing NIFᵥₐᵣ₋₁ with NIF_{ref-1} against a CSP reference database.

In embodiments related to the second embodiment, the sample splice site may be a donor splice site. In certain embodiments, the sample splice site sequence comprises 4 to 12 nucleotides of a donor splice site. In certain embodiments, the sample splice site sequence comprises 4, 5, 6, 7, 8, 9, 10, 11, or 12 consecutive nucleotides of a donor splice site. In certain embodiments, the sample splice site sequence comprises 4 to 15 nucleotides of a donor splice site. In certain embodiments, the sample splice site sequence comprises 4, 5, 6, 7, 8, 9, 10, 11, 12, 13, 14 or up to 15 consecutive nucleotides of a donor splice site. In certain embodiments, the sample splice site sequence comprises 30 or more nucleotides of a donor splice site. In certain embodiments, the sample splice site sequence comprises 30 or more consecutive nucleotides of a donor splice site. In certain embodiments, the sample splice site sequence comprises 9 consecutive nucleotides of a donor splice site. In certain embodiments, the method is repeated with one or more sample splice site sequences comprised in the same sample splice site; wherein a risk of abnormal splicing is determined by comparing each NIFᵥₐᵣ₋ₓ with a corresponding NIF_{ref-x} against a CSP reference database. In certain embodiments the sample splice site is a donor splice site, the method is repeated with a second sample donor splice site sequence comprised in the same sample splice site and a corresponding second reference donor splice site sequence, and NIFᵥₐᵣ₋₂ and NIF_{ref-2} are determined. In certain embodiments, the sample splice site is a donor splice site, the method is repeated with up to five additional sample donor splice site sequences comprised in the same sample splice site, and five respective donor reference splice site sequences, wherein NIFᵥₐᵣ₋₂, NIFᵥₐᵣ₋₃, NIFᵥₐᵣ₋₄, NIFᵥₐᵣ₋₅, up to NIFᵥₐᵣ₋₆, corresponding to NIFᵥₐᵣ for each of the second, third, fourth, fifth, and up to sixth sample donor splice site sequence, and NIF_{ref-2}, NIF_{ref-3}, NIF_{ref-4}, NIF_{ref-5}, and up to NIF_{ref-6}, corresponding to NIF_{ref} for each of the second, third, fourth, fifth, and up to sixth reference donor splice site sequences. In certain embodiments, the splice site is a donor splice site, and the steps are repeated with up to five additional sample donor splice site sequences comprised in the same sample splice site, wherein each sample donor splice site sequence comprises 9 non-identical consecutive nucleotides of the donor splice site, and wherein the sample donor splice site sequences may comprise overlapping consecutive nucleotides of the sample donor splice site. In a related embodiment comprising at least six sample splice site sequences from a sample splice site, the sample splice site sequences correspond to at least nucleotide positions E⁻⁵ to D⁺⁴, E⁻⁴ to D⁺⁵, E⁻³ to D⁺⁶, E⁻² to D⁺⁷, E⁻¹ to D⁺⁵, and D⁺¹ to D⁺⁹ of a donor splice site. In a related embodiment comprising at least four sample splice site sequences from a sample splice site, the sample splice site sequences correspond to at least nucleotide positions E⁻⁴ to D⁺⁵, E⁻³ to D⁺⁶, E⁻² to D⁺⁷ and E⁻¹ to D⁺⁸ of a donor splice site.

In embodiments related to the second embodiment, the sample splice site is a donor splice site. In certain embodiments, the sample splice site sequence comprises 6 to 15 nucleotides of a donor splice site. In certain embodiments, the sample splice site sequence comprises 12 consecutive nucleotides of a donor splice site that is analysed as a collective of multiple, overlapping donor reference splice site sequences, wherein the median of NIFᵥₐᵣ₋₁, NIFᵥₐᵣ₋₂, NIFᵥₐᵣ₋₃, NIFᵥₐᵣ₋₄ and up to NIFᵥₐᵣ₋₆, corresponding to NIFᵥₐᵣ for each of the first, second, third, fourth and up to sixth sample donor splice site sequences, is compared with the median of NIF_{ref-1}, NIF_{ref-2}, NIF_{ref-3}, NIF_{ref-4} and up to NIF_{ref-6}, corresponding to NIF_{ref} for each of the first, second, third, fourth and up to sixth reference donor splice site sequences. In certain embodiments, the sample splice site is a donor splice site of 12 nucleotides divided into four sample splice site sequences comprised of 9 non-identical sequences of consecutive nucleotides corresponding to nucleotide positions E⁻⁴ to D⁺⁵, E⁻³ to D⁺⁶, E⁻² to D⁺⁷ and E⁻¹ to D⁺⁸ of a donor splice site. The median NIFᵥₐᵣ₋ₓ is calculated as median (NIFᵥₐᵣ₋₁; NIFᵥₐᵣ₋₂; NIFᵥₐᵣ₋₃; NIFᵥₐᵣ₋₄) and the median NIF_{ref-x} is calculated as median (NIF_{ref-1}; NIF_{ref-2}; NIF_{ref-3}; NIF_{ref-4}), wherein each analagous variant and reference donor splice site sequence NIFᵥₐᵣ₋₁ and NIF_{ref-1}, NIFᵥₐᵣ₋₂ and NIF_{ref-2}, NIFᵥₐᵣ₋₃ and NIF_{ref-3}, NIFᵥₐᵣ₋₄ and NIF_{ref-4} originate from the same corresponding region of a gene and respectively encompass nucleotide positions E⁻⁴ to D⁺⁵, E⁻³ to D⁺⁶, E⁻² to D⁺⁷ and E⁻¹ to D⁺⁸.

In further embodiments related to the second embodiment, the sample splice site is a donor splice site. In certain embodiments, the sample splice site sequence comprises 6 to 15 nucleotides of a donor splice site. In certain embodiments, the sample splice site sequence comprises 12 consecutive nucleotides of a donor splice site that is analysed as a collective of multiple, overlapping donor reference splice site sequences, wherein the median percentile NIFᵥₐᵣ₋ₓ is calculated as median (NIFᵥₐᵣ₋₁ percentile; NIFᵥₐᵣ₋₂ percentile; percentile of NIFᵥₐᵣ₋₃ percentile; NIFᵥₐᵣ₋₄ percentile) wherein a median percentile NIFᵥₐᵣ₋ₓ of 0 (zero) for any sample donor splice site sequence indicates that the sample donor splice site is abnormal. For example, a hypothetical site with percentile NIFᵥₐᵣ₋₁=0.2499, percentile NIFᵥₐᵣ₋₂=0.5904, percentile NIFᵥₐᵣ₋₃=0.7172, percentile NIFᵥₐᵣ₋₄=0.9065 has a median percentile NIFvar-x of 0.6538. For the same hypothetical example, a site with percentile NIFᵥₐᵣ₋₁=0.0077, percentile NIFᵥₐᵣ₋₂=0.0295, percentile NIFᵥₐᵣ₋₃=0.0493, percentile NIFᵥₐᵣ₋₄=0.0635 has a median percentile NIFvar-x of 0.0394 Therefore, the net percentile change in median NIF for the hypothetical sample splice site is 0.0602 (0.0394/0.6538).

In embodiments related to the second embodiment, provided is a method of identifying an abnormal splice site in a sample splice site from a subject, said method comprising:
a) obtaining a sample splice site sequence from the subject and determining the median NIFᵥₐᵣ₋ₓ. In certain embodiments, the sample splice site sequence comprises 12 nucleotides of a donor splice site. In a related embodiment, NIFᵥₐᵣ₋₁, NIFᵥₐᵣ₋₂, NIFᵥₐᵣ₋₃, NIFᵥₐᵣ₋₄ comprise four sample splice site sequences of nine consecutive nucleotides from a sample splice site and the median NIFᵥₐᵣ₋ₓ is calculated as [median(NIFᵥₐᵣ₋₁; NIFᵥₐᵣ₋₂; NIFᵥₐᵣ₋₃; NIFᵥₐᵣ₋₄)].
b) obtaining a reference splice site sequence; wherein the reference splice site sequence and the sample splice site sequence each originate from the same corresponding region of a gene. In certain embodiments, the reference splice site sequence comprises 12 nucleotides of a donor splice site. In a related embodiment, NIF_{ref-1}, NIF_{ref-2}, NIF_{ref-3} and NIF_{ref-4} comprise four reference splice site sequences of nine consecutive nucleotides from a reference splice site and the median NIF_{ref-x} is calculated as [median (NIF_{ref-1}; NIF_{ref-2}; NIF_{ref-3}; NIF_{ref-4})].
c) determining a risk of abnormal splicing for the sample splice site by comparing the median NIFᵥₐᵣ₋ₓ with the median NIF_{ref-x} against a Clinical Splice Predictor (CSP) reference database.

In further embodiments related to the second embodiment, provided is a method of identifying an abnormal splice site in a sample splice site from a subject, said method comprising:
a) obtaining a sample splice site sequence from the subject, determining the median percentile NIFᵥₐᵣ₋ₓ calculated as [median(percentile NIFᵥₐᵣ₋₁; percentile NIFᵥₐᵣ₋₂; percentile NIFᵥₐᵣ₋₃; percentile NIFᵥₐᵣ₋₄)].
b) obtaining a reference splice site sequence; wherein the reference splice site sequence and the sample splice site sequence each originate from the same corresponding region of a gene. Determining the median percentile NIF_{ref-x} calculated as [median (percentile NIF_{ref-1}; percentile NIF_{ref-2}; percentile NIF_{ref-3}; percentile NIF_{ref-4})].
c) determining a risk of abnormal splicing for the sample splice site by comparing the net percentile change in median NIF between the sample splice and the reference splice site against a Clinical Splice Predictor (CSP) reference database.

In further embodiments related to the second embodiment, the use of median NIFᵥₐᵣ₋ₓ described in Section [0019] and Section [0021] may be substituted for mean NIFᵥₐᵣ₋ₓ calculated as mean (NIFᵥₐᵣ₋₁; NIFᵥₐᵣ₋₂; NIFᵥₐᵣ₋₃; NIFᵥₐᵣ₋₄).

In further embodiments related to the second embodiment, the use of median NIFᵥₐᵣ₋ₓ converted to a percentile value described in Section [0020] and Section [0022] may be substituted for mean percentile NIFᵥₐᵣ₋ₓ.

In a third embodiment, provided is a method of identifying an abnormal splice site in a sample splice site from a subject, said method comprising:

(a) obtaining a first sample splice site sequence comprised in the sample splice site from the subject;
(b) determining a clinical classification(s) associated with the nucleotide sequence of the first sample splice site sequence;
(c) determining a clinical classification(s) associated with the nucleotide sequence of the first reference splice site sequence; and
(d) determining a risk of abnormal splicing for the sample splice site by assessing the clinical classification(s) associated with the nucleotide sequence of the first sample splice site sequence determined in step (c).

In an embodiment related to the third embodiment, provided is a method of identifying an abnormal splice site in a sample splice site from a subject, said method comprising:

(a) obtaining a first sample splice site sequence comprised in the sample splice site from the subject;
(b) obtaining a first reference splice site sequence; wherein the first reference splice site sequence and the first sample splice site sequence each originate from the same corresponding region of a gene;
(c) determining a clinical classification(s) associated with the nucleotide sequence of the first sample splice site sequence;
(d) determining a clinical classification(s) associated with the nucleotide sequence of the first reference splice site sequence; and
(e) determining a risk of abnormal splicing for the sample splice site by assessing the clinical classification(s) associated with the nucleotide sequence of the first sample splice site sequence determined in step (c) and the clinical classification(s) associated with the nucleotide sequence of the first reference splice site sequence determined in step (d).

In further embodiments related to the third embodiment, the sample splice site may be a donor splice site. In certain embodiments, the sample splice site sequence comprises 4 to 12 nucleotides of a donor splice site. In certain embodiments, the sample splice site sequence comprises 4, 5, 6, 7. 8, 9, 10, 11, or 12 consecutive nucleotides of a donor splice site. In certain embodiments, the sample splice site sequence comprises 4 to 15 nucleotides of a donor splice site. In certain embodiments, the sample splice site sequence comprises 4, 5, 6, 7, 8, 9, 10, 11, 12, 13, 14 or up to 15 consecutive nucleotides of a donor splice site. In certain embodiments, the sample splice site sequence comprises 30 or more nucleotides of a donor splice site. In certain embodiments, the sample splice site sequence comprises 30 or more consecutive nucleotides of a donor splice site. In certain embodiments, the sample splice site sequence comprises 9 consecutive nucleotides of a donor splice site. In certain embodiments comprising determining a clinical classification(s) associated with a sample splice site sequence, and (optionally) a reference splice site sequence, the sample splice site is a donor splice site, the steps are repeated with up to five sample splice site sequences comprised in the same sample splice site and (optionally) corresponding respective reference splice site sequences, and determining a risk of abnormal splicing for the sample splice site includes assessing the clinical classification(s) associated with the nucleotide sequence of each sample splice site sequence and (optionally) each corresponding reference splice site sequence. In embodiments related to the third embodiment, a clinical classification(s) as recited may be determined by querying a CSP database for the respective nucleotide sequence of the sample splice site sequence and/or the nucleotide sequence of the corresponding reference splice site sequence. A risk of abnormal splicing for a sample splice site may be determined by considering the number of times the nucleotide sequence of each sample splice site sequence has been identified as an abnormal splice site.

In an embodiment related to the third embodiment, provided is a method of identifying an abnormal splice site in a sample splice site from a subject, said method comprising:
(a) obtaining a sample splice site sequence from the subject and deriving median NIFᵥₐᵣ₋ₓ;
(b) obtaining a reference splice site sequence and deriving median NIF_{ref-x}; wherein the reference splice site sequence and the sample splice site sequence each originate from the same corresponding region of a gene;
(c) obtaining other variant splice site sequence(s) from the CSP reference database that affect the same donor splice site from the same corresponding region of a gene and derive the median NIFᵥₐᵣ₋ₓ;
(d) calculating the net change in median NIFᵥₐᵣ₋ₓ/median NIF_{ref-x} for the sample splice site sequence and the other variant splice site sequence(s) from the CSP reference database that affect the same donor splice site; and
(e) determining a risk of abnormal splicing for the sample splice site by assessing the clinical classification(s) associated with a net change in median NIFᵥₐᵣ₋ₓ/median NIF_{ref-x} for other variant splice site sequence(s) from the CSP reference database that affect the same donor splice site as determined in step (d).

In a further embodiment related to the third embodiment, provided is a method of identifying an abnormal splice site in a sample splice site from a subject, said method comprising:
(a) obtaining a sample splice site sequence from the subject, deriving median NIFᵥₐᵣ₋ₓ and converting this to a percentile value;
(b) obtaining a reference splice site sequence, deriving median NIF_{ref-x} and converting this to a percentile value; wherein the reference splice site sequence and the sample splice site sequence each originate from the same corresponding region of a gene;
(c) obtaining other variant splice site sequence(s) from the CSP reference database that affect the same donor splice site from the same corresponding region of a gene, deriving the median NIFᵥₐᵣ₋ₓ and converting this to a percentile value;
(d) calculating the net change in the percentile median NIFᵥₐᵣ₋ₓ/percentile median NIF_{ref-x} for the sample splice site sequence, as well as the other variant splice site sequence(s) from the CSP reference database that affect the same donor splice site; and
(e) determining a risk of abnormal splicing for the sample splice site by assessing the clinical classification(s) associated with a net change in percentile median NIFᵥₐᵣ₋ₓ/percentile median NIF_{ref-x} for other variant splice site sequence(s) from the CSP reference database that affect the same donor splice site as determined in step (d).

In further embodiments, calculation of the median NIFᵥₐᵣ₋ₓ described in Section [0028] may be substituted for the mean NIFᵥₐᵣ₋ₓ.

In further embodiments, calculation of the median percentile NIFᵥₐᵣ₋ₓ in Section [0029 may be substituted for the mean percentile NIFᵥₐᵣ₋ₓ.

In a fourth embodiment, provided is a method of identifying an abnormal splice site in a sample splice site from a subject, said method comprising:

(a) obtaining a first sample splice site sequence comprised in the sample splice site from the subject;
(b) determining a measure of Native Intron Frequency of the first sample splice site sequence (NIFᵥₐᵣ₋₁);
(c) determining a Percentile (NIFᵥₐᵣ₋₁) of the first sample splice site sequence;
(d) determining a measure of Native Intron Frequency of a first reference splice site sequence (NIF_{ref-1}); wherein the first reference splice site sequence and the first sample splice site sequence originate from the same corresponding region of a gene;
(e) determining a Percentile (NIF_{ref-1}) of the first reference splice site sequence;
(f) calculating a lower bound and an upper bound for Percentile (NIFᵥₐᵣ₋₁) and calculating a lower bound and an upper bound for Percentile (NIF_{ref-1});
(g) determining a range of NIF-shift by comparing the lower and upper bounds for Percentile (NIFᵥₐᵣ₋₁) with the lower and upper bounds for Percentile (NIF_{ref-1}) calculated in (f);
(h) identifying (a) similar NIF-shift variant(s), wherein a similar NIF-shift variant refers to a splice site sequence with a NIF-shift within the range of NIF-shift determined in (g);
(i) determining (a) clinical classification(s) associated with each similar NIF-shift variant identified in step (h); and
(j) determining a risk of abnormal splicing for the sample splice site by assessing the clinical classification(s) determined in step (i) for each similar NIF-shift variant identified in step (h).

In an embodiment related to the fourth embodiment, provided is a method of identifying an abnormal splice site in a sample splice site from a subject, said method comprising:

(a) obtaining a first sample splice site sequence comprised in the sample splice site from the subject;
(b) determining a measure of Native Intron Frequency of the first sample splice site sequence (NIFᵥₐᵣ₋₁);
(c) determining a measure of Native Intron Frequency of a first reference splice site sequence (NIF_{ref-1}); wherein the first reference splice site sequence and the first sample splice site sequence originate from the same corresponding region of a gene;
(d) calculating a lower bound and an upper bound for NIFᵥₐᵣ₋₁ and calculating a lower bound and an upper bound for NIF_{ref-1};
(e) determining a range of NIF-shift by comparing the lower and upper bounds for NIFᵥₐᵣ₋₁ with the lower and upper bounds for NIF_{ref-1} calculated in (d);
(f) identifying (a) similar NIF-shift variant(s), wherein a NIF-shift variant refers to a splice site sequence with a NIF-shift within the range of NIF-shift determined in (e);
(g) determining (a) clinical classification(s) associated with each similar NIF-shift variant identified in step (f); and
(h) determining the risk of abnormal splicing for the sample splice site by assessing the clinical classification(s) determined in step (g) for each similar NIF-shift variant identified in step (f).

In embodiments related to the fourth embodiment, the sample splice site may be a donor splice site. In certain embodiments, the sample splice site sequence comprises 4 to 12 nucleotides of a donor splice site. In certain embodiments, the sample splice site sequence comprises 4, 5, 6, 7, 8, 9, 10, 11, or 12 consecutive nucleotides of a donor splice site. In certain embodiments, the sample splice site sequence comprises 4 to 15 nucleotides of a donor splice site. In certain embodiments, the sample splice site sequence comprises 4, 5, 6, 7, 8, 9, 10, 11, 12, 13, 14 or up to 15 consecutive nucleotides of a donor splice site. In certain embodiments, the sample splice site sequence comprises 30 or more nucleotides of a donor splice site. In certain embodiments, the sample splice site sequence comprises 30 or more consecutive nucleotides of a donor splice site. In certain embodiments, the sample splice site sequence comprises 9 consecutive nucleotides of a donor splice site. In certain embodiments, the sample splice site is a donor splice site, the steps are repeated with up to five sample splice site sequences comprised in the same sample splice site and corresponding reference splice site sequences, and the method includes assessing the clinical classification(s) associated with each similar NIF-shift variant identified. In certain embodiments, the sample splice site is a donor splice site, and the steps are repeated with up to five additional sample donor splice site sequences, wherein each sample donor splice site sequence comprises 9 non-identical consecutive nucleotides of the same sample donor splice site, and wherein the sample donor splice site sequences may comprise overlapping consecutive nucleotides of the donor splice sites. In a related embodiment comprising at least six sample splice site sequences from the same sample splice site, the sample splice site sequences correspond to at least nucleotide positions E⁻⁵ to D⁺⁴, E⁻⁴ to D⁺⁵, E⁻³ to D⁺⁶, E⁻² to D⁺⁷, E⁻¹ to D⁺⁸, and D⁺¹ to D⁺⁹ of a donor splice site. In a related embodiment comprising at least four sample splice site sequences from the same sample splice site, the sample splice site sequences correspond to at least nucleotide positions E⁻⁴ to D⁺⁵, E⁻³ to D⁺⁶, E⁻² to D⁺⁷ and E⁻¹ to D⁺⁸ of a donor splice site.

In embodiments related to the fourth embodiment, suitable upper and lower bounds of a NIF or Percentile (NIF) may be calculated based on a percentage (e.g., 10%, 5%, 2.5%, 2%) of a logarithmic distribution of NIF or Percentile (NIF), median NIF or Percentile median NIF, mean NIF or Percentile mean NIF, wherein the upper and lower bounds are whole numbers rounded to the nearest whole numbers.

In a fifth embodiment, provided is a method of identifying an abnormal splice site in a sample splice site from a subject, said method comprising

(a) obtaining a first sample splice site sequence comprised in the sample splice site from the subject;
(b) determining a measure of Native Intron Frequency of the first sample splice site sequence (NIFᵥₐᵣ₋₁);
(c) determining a Percentile (NIFᵥₐᵣ₋₁) of the first sample splice site sequence;
(d) determining a measure of Native Intron Frequency of a first reference splice site sequence (NIF_{ref-1}); wherein the first reference splice site sequence and the first sample splice site sequence each originate from the same corresponding region of a gene;
(e) determining a Percentile (NIF_{ref-1}) of the first reference splice site sequence; (f) determining (a) clinical classification(s) associated with the nucleotide sequence of the first sample splice site sequence;
(g) optionally determining (a) clinical classification(s) associated with the nucleotide sequence of the first reference splice site sequence;
(h) calculating a lower bound and an upper bound for Percentile (NIFᵥₐᵣ₋₁) and calculating a lower bound and an upper bound for Percentile (NIF_{ref-1});
(i) determining a range of NIF-shift by comparing the lower and upper bounds for Percentile (NIFᵥₐᵣ₋₁) and the lower and upper bounds for Percentile (NIF_{ref-1}) calculated in (h);
(j) identifying (a) similar NIF-shift variant(s), wherein a similar NIF-shift variant refers to a splice site sequence with a NIF-shift within the range of NIF-shift determined in (i);
(k) determining (a) clinical classification(s) associated with each similar NIF-shift variant identified in step (j); and
(l) determining a risk of abnormal splicing for the sample splice site by (1) comparing the Percentile (NIFᵥₐᵣ₋₁) with the Percentile (NIF_{ref-1}) against a CSP reference database, (2) assessing the clinical classification(s) associated with the nucleotide sequence of the first sample splice site sequence determined in step (f); and (3) assessing the clinical classification determined in step (k) for each NIF-shift variant identified in step (j).

In a related embodiment, step (g) is carried out; and step (l) may further comprise as part of (2), analysing the clinical classification(s) associated with the nucleotide sequence of the first reference splice site sequence determined in step (g).

In an embodiment related to the fifth embodiment, provided is a method of identifying an abnormal splice site in a sample splice site from a subject, said method comprising

(a) obtaining a first sample splice site sequence comprised in the sample splice site from the subject;
(b) determining a measure of Native Intron Frequency of the first sample splice site sequence (NIFᵥₐᵣ₋₁);
(c) determining a measure of Native Intron Frequency of a first reference splice site sequence (NIF_{ref-1}); wherein the first reference splice site sequence and the first sample splice site sequence each originate from the same corresponding region of a gene;
(d) determining (a) clinical classification(s) associated with the nucleotide sequence of the first sample splice site sequence;
(e) optionally determining (a) clinical classification(s) associated with the nucleotide sequence of the first reference splice site sequence;
(f) calculating a lower bound and an upper bound for NIFᵥₐᵣ₋₁ and calculating a lower bound and an upper bound for NIF_{ref-1};
(g) determining a range of NIF-shift by comparing the lower and upper bounds for NIFᵥₐᵣ₋₁ and the lower and upper bounds for NIF_{ref-1} calculated in (f);
(h) identifying (a) similar NIF-shift variant(s), wherein a similar NIF-shift variant refers to a splice site sequence with a NIF-shift within the range of NIF-shift determined in (g);
(i) determining (a) clinical classification(s) associated with each similar NIF-shift variant identified in step (h); and
(j) determining a risk of abnormal splicing for the sample splice site by (1) comparing the NIFᵥₐᵣ₋₁ with the NIF_{ref-1} against a CSP reference database, (2) assessing the clinical classification(s) associated with the nucleotide sequence of the first sample splice site sequence determined in step (d); and (3) assessing the clinical classification determined in step (i) for each similar NIF-shift variant identified in step (h).

In a related embodiment, step (e) is carried out; and step (j) may further comprise as part of (2), analysing the clinical classification(s) associated with the nucleotide sequence of the first reference splice site sequence determined in step (e).

In further embodiments related to the fifth embodiment, the sample splice site may be a donor splice site. In certain embodiments, the sample splice site sequence comprises 4 to 12 nucleotides of a donor splice site. In certain embodiments, the sample splice site sequence comprises 4, 5, 6, 7, 8, 9, 10, 11, or 12 consecutive nucleotides of a donor splice site. In certain embodiments, the sample splice site sequence comprises 4 to 15 nucleotides of a donor splice site. In certain embodiments, the sample splice site sequence comprises 4, 5, 6, 7, 8, 9, 10, 11, 12, 13, 14 or up to 15 consecutive nucleotides of a donor splice site. In certain embodiments, the sample splice site sequence comprises 30 or more nucleotides of a donor splice site. In certain embodiments, the sample splice site sequence comprises 30 or more consecutive nucleotides of a donor splice site. In certain embodiments, the sample splice site sequence comprises 9 consecutive nucleotides of a donor splice site. In certain embodiments, the sample splice site is a donor splice site, and the method is repeated with up to five sample splice site sequences comprised in the same sample splice site and corresponding respective reference splice site sequences. In certain embodiments, the splice site is a donor splice site, and the steps are repeated with up to five additional sample donor splice site sequences comprised in the same sample splice site, wherein each sample donor splice site sequence comprises 9 non-identical consecutive nucleotides of the donor splice site, and wherein the sample donor splice site sequences may comprise overlapping consecutive nucleotides of the donor splice site. In a related embodiment comprising at least six sample splice site sequences from the same sample splice site, the sample splice site sequences correspond to at least nucleotide positions E⁻⁵ to D⁺⁴, E⁻⁴ to D⁺⁵, E⁻³ to D⁺⁶, E⁻² to D⁺⁷, E⁻¹ to D⁺⁸, and D⁺¹ to D⁺⁹ of a donor splice site. In a related embodiment comprising at least four sample splice site sequences from the same sample splice site, the sample splice site sequences correspond to at least nucleotide positions E⁻⁴ to D⁺⁵, E⁻³ to D⁺⁶, E⁻² to D⁺⁷ and E⁻¹ to D⁺⁸ of a donor splice site.

In an embodiment related to the fifth embodiment, provided is a method of identifying an abnormal splice site in a sample splice site from a subject, said method comprising:
a) obtaining a sample splice site sequence from the subject;
b) determining a measure of the median Native Intron Frequency of the sample splice site sequence (median; NIFᵥₐᵣ₋ₓ)
c) determining a Percentile value for the median NIFᵥₐᵣ₋ₓ of the sample splice site sequence;
d) determining a measure of the median Native Intron Frequency of the reference splice site sequence (median; NIF_{ref-x}); wherein the reference splice site sequence and the sample splice site sequence originate from the same corresponding region of a gene;
e) determining a Percentile value for the median NIF_{ref-x} of the reference splice site sequence;
f) calculating a lower bound and an upper bound for Percentile (median NIFᵥₐᵣ₋ₓ) and calculating a lower bound and an upper bound for Percentile (median NIF_{ref-x});
g) determining a range of NIF-shift by comparing the lower and upper bounds for Percentile (median NIFᵥₐᵣ₋ₓ) with the lower and upper bounds for Percentile (median NIF_{ref-x}) calculated in (f);
h) identifying (a) similar NIF-shift variant(s), wherein a similar NIF-shift variant refers to a splice site sequence with a NIF-shift within the range of NIF-shift determined in (g);
i) determining (a) clinical classification(s) associated with each similar NIF-shift variant identified in step (h); and
j) determining a risk of abnormal splicing for the sample splice site by assessing the clinical classification(s) determined in step (i) for each similar NIF-shift variant identified in step (h).

In an embodiment related to the fifth embodiment, provided is a method of identifying an abnormal splice site in a sample splice site from a subject, said method comprising:
a) obtaining a sample splice site sequence from the subject;
b) determining a measure of the mean Native Intron Frequency of the sample splice site sequence (mean; NIFᵥₐᵣ₋ₓ);
c) determining a Percentile value for the mean NIFᵥₐᵣ₋ₓ of the sample splice site sequence;
d) determining a measure of the mean Native Intron Frequency of the reference splice site sequence (mean; NIF_{ref-x}); wherein the reference splice site sequence and the sample splice site sequence originate from the same corresponding region of a gene;
e) determining a Percentile value for the mean NIF_{ref-x} of the reference splice site sequence;
f) calculating a lower bound and an upper bound for Percentile (mean NIFᵥₐᵣ₋ₓ) and calculating a lower bound and an upper bound for Percentile (mean NIF_{ref-x});
g) determining a range of NIF-shift by comparing the lower and upper bounds for Percentile (mean NIFᵥₐᵣ₋ₓ) with the lower and upper bounds for Percentile (mean NIF_{ref-x}) calculated in (f);
h) identifying (a) similar NIF-shift variant(s), wherein a similar NIF-shift variant refers to a splice site sequence with a NIF-shift within the range of NIF-shift determined in (g);
i) determining (a) clinical classification(s) associated with each similar NIF-shift variant identified in step (h); and
j) determining a risk of abnormal splicing for the sample splice site by assessing the clinical classification(s) determined in step (i) for each similar NIF-shift variant identified in step (h).

In a sixth embodiment provided is a method of identifying an abnormal splice site in a sample splice site from a subject, said method comprising:
a) obtaining a sample splice site sequence from the subject;
b) determining a measure of the median Native Intron Frequency of the sample splice site sequence (median; NIFᵥₐᵣ₋ₓ);
c) determining a measure of the median Native Intron Frequency of the reference splice site sequence (median; NIF_{ref-x}); wherein the first reference splice site sequence and the sample splice site sequence originate from the same corresponding region of a gene;
a) determining a measure of the median Native Intron Frequency of a cryptic donor splice site(s) (median NIF_{CSS-x}) within 150 nucleotides of the reference splice site (plus or minus 150 nucleotides). In certain embodiments, a cryptic donor splice site sequence is defined by any GT (or GC) within 150 nucleotides of a reference splice site, wherein the GT (or GC) represent the nucleotides comprising the essential splice site at positions D⁺¹ and D⁺². In certain embodiments, a cryptic donor splice site sequence comprises 4, 5, 6, 7, 8, 9, 10, 11, 12 or up to 15 consecutive nucleotides of a cryptic donor splice site. In certain embodiments, a cryptic donor splice site sequence consists of 12 nucleotides comprised of four overlapping sequences of nine consecutive nucleotides, corresponding to nucleotide positions E⁻⁴ to D⁺⁵, E⁻³ to D⁺⁶, E⁻² to D⁺⁷ and E⁻¹ to D⁺⁸, wherein the GT (or GC) represent the nucleotides comprising the essential splice site at positions D⁺¹ and D⁺² of the cryptic donor splice site;
d) determining a risk of abnormal splicing for the sample splice site by assessing the median NIFᵥₐᵣ₋ₓ determined in (b), relative to median NIF_{ref-x} determined in (c);
e) determining a risk of abnormal splicing for the sample splice site by assessing the median NIFᵥₐᵣ₋ₓ determined in (b), relative to median NIF_{css-x} determined in (d);
f) determining a risk of abnormal splicing for the reference splice site by assessing the median NIF_{ref-x} determined in (c), relative to median NIF_{css-x} determined in (d).

In an embodiment related to the sixth embodiment is a method of identifying an abnormal splice site in a sample splice site from a subject, said method comprising:
b) obtaining a sample cryptic donor splice site sequence from the subject. In certain embodiments, a cryptic donor splice site sequence is defined by any GT (or GC) within 150 nucleotides of a reference splice site, wherein the GT (or GC) represent the nucleotides comprising the essential splice site at positions D⁺¹ and D⁺². In certain embodiments, a cryptic donor splice site sequence comprises 4, 5, 6, 7, 8, 9, 10, 11, 12 or up to 15 consecutive nucleotides of a cryptic donor splice site. In certain embodiments, a cryptic donor splice site sequence consists of 12 nucleotides comprised of four overlapping sequences of nine consecutive nucleotides, corresponding to nucleotide positions E⁻⁴ to D⁺⁵, E⁻³ to D⁺⁶, E⁻² to D⁺⁷ and E⁻¹ to D⁺⁸, wherein the GT (or GC) represent the nucleotides comprising the essential splice site at positions D⁺¹ and D⁺² of the cryptic donor splice site;
c) determining a measure of the median Native Intron Frequency of the reference splice site sequence (median; NIF_{ref-x}), whereby the reference splice site is correctly positioned at the exon-intron junction and the cryptic donor splice site lies within 150 nucleotides upstream or downstream of the same exon-intron junction. In certain embodiments, the reference splice site sequence comprises 4, 5, 6, 7, 8, 9, 10, 11, 12 or up to 15 consecutive nucleotides of a donor splice site. In certain embodiments, the reference splice site sequence consists of 12 nucleotides comprised of four overlapping sequences of nine consecutive nucleotides, corresponding to nucleotide positions E⁻⁴ to D⁺⁵, E⁻³ to D⁺⁶, E⁻² to D⁺⁷ and E⁻¹ to D⁺⁸, wherein the GT (or GC) represent the nucleotides comprising the essential splice site at positions D⁺¹ and D⁺² of the reference donor splice site;
d) determining a risk of abnormal splicing for the reference splice site by assessing the median NIF_{ref-x} determined in (c), relative to median NIF_{css-x} determined in (a).

Methods of identifying an abnormal splice site in a sample splice site further relate to combinations of any method or any embodiment herein disclosed, including combinations of embodiments related to the first, second, and third embodiments or embodiments related to the first, second and fourth embodiments. Combinations of embodiments related to the first, second, third, and/or fourth embodiments are also envisioned. Certain embodiments relate to a combination of the second, third, fourth, fifth and sixth embodiments. Certain embodiments relate to a combination of the second and fourth embodiments. It will be appreciated that in relation to combinations of embodiments, there is no requirement to carry out the combination of embodiments and/or steps of an embodiment in any particular order. Methods comprising determining a measure of frequency of a sample splice site in combination with a previous classification factor and/or similar splice site frequency shift factor (similar NIF-shift factor) and/or competitive cryptic splice site factor are envisioned.

### Definitions

Unless the context clearly requires otherwise, throughout the description and the claims, the words “comprise”, “comprising” and the like are to be construed in an inclusive sense as opposed to an exclusive or exhaustive sense; that is to say, in the sense of “including, but not limited to”.

As used herein, the term “about” can mean within 1 or more standard deviation per the practice in the art. Alternatively, “about” can mean a range of up to 20%, up to 10%, or up to 5%. In certain embodiments, “about” can mean to 5%.

As used herein and in the appended claims, the singular form of “a”, “an”, and “the” may include the plural referents unless the context clearly dictates otherwise. It is further noted that the claims may be drafted to exclude any optional element.

As used herein, the term “splice site” refers to a consensus element in an exon and/or an intron of genomic DNA, including, but not limited to, a donor splice site, a branch site, and an acceptor splice site.

As used herein, the term “splice site sequence” refers to a region of nucleotides in a splice site. A splice site sequence may comprise one or more regions of consecutive nucleotides of a sample splice site. In certain embodiments, a splice site sequence may comprise one or more regions of consecutive nucleotides with one or more groups consisting of a single nucleotide. A splice site sequence may comprise nucleotides from an exon, an intron, or both an exon and an intron. In one embodiment, a splice site sequence comprises or consists of nucleotides of an intron. In one embodiment, a splice site sequence is a donor splice site sequence comprising nucleotides of an exon and intron.

As used herein, the term “donor splice site” refers to a consensus element located near the 5′ end of an intron and also referred to as an “exon-intron boundary”. In one embodiment, a donor splice site comprises or consists of nucleotides of an intron. In one embodiment, a donor splice site comprises nucleotides of an exon-intron boundary comprising at least one nucleotide from the 3′ end of an exon and at least 4 nucleotides of the 5′ end of an intron. In one embodiment, a “donor splice site” comprises the five-3′end nucleotides of the exon (E⁻⁵ to E⁻¹) and the eight-5′end nucleotides of the intron (D⁺¹ to D⁺⁸). In one embodiment, a “donor splice site” comprises the five-3′end nucleotides of the exon (E⁻⁵ to E⁻¹) and the nine-5′end nucleotides of the intron (D⁺¹ to D⁺⁹). In certain embodiments, the GT (or GC) nucleotides corresponding to the essential splice site that encompass the first two nucleotides of the intron, are denoted as positions D⁺¹ and D⁺² of the donor splice site.

As used herein, the term “donor splice site sequence” refers to nucleotides comprised in a donor splice site. In certain embodiments, a donor splice site sequence comprises 4 to 12 nucleotides of a donor splice site. In one embodiment, a donor splice site sequence comprises 4, 5, 6, 7, 8, 9, 10, 11, or 12 consecutive nucleotides of a donor splice site. In certain embodiments, the sample splice site sequence comprises 4 to 15 nucleotides of a donor splice site. In certain embodiments, the sample splice site sequence comprises 4, 5, 6, 7, 8, 9, 10, 11, 12, 13, 14 or up to 15 consecutive nucleotides of a donor splice site. In certain embodiments, the sample splice site sequence comprises 30 or more nucleotides of a donor splice site. In certain embodiments, the sample splice site sequence comprises 30 or more consecutive nucleotides of a donor splice site. In certain embodiments, a donor splice site sequence comprises 9 consecutive nucleotides of a donor splice site. In certain embodiments, a donor splice site sequence comprises or consists of nucleotides of an intron. In certain embodiments, a donor splice site sequence comprises at least one nucleotide of an exon. In certain embodiments, a donor splice site sequence comprises nucleotides of an exon and nucleotides of an intron.

As used herein, the term “essential donor splice site” refers to the first two nucleotides of the intron, denoted as positions D⁺¹ (first nucleotide of the intron) and D⁺² (second nucleotide of the intron). The skilled person will be familiar that the essential donor splice site is comprised of GT (guanine, thymine) nucleotides at the first and second position of the intron for ˜99% of human introns.

As used herein, the term “branch site” refers to a consensus element located near the 3′ end of an intron and is upstream of the polypyrimidine tract.

As used herein, the term polypyrimidine tract refers to a consensus element located near the 3′ end of an intron that is enriched in pyrimidine nucleotides cytosine (C) and thymine (T).

As used herein, the term “branch site sequence” refers to nucleotides comprised in a branch site. In certain embodiments, a branch site sequence comprises 6 to 9 nucleotides of a branch site that includes the branchpoint A (adenosine or adenine). In certain embodiments, a branch site sequence comprises 6, 7, 8, or 9 consecutive nucleotides of a branch site. In certain embodiments, a branch splice site sequence comprises 7 consecutive nucleotides of a branch site.

As used herein, the term “acceptor splice site” refers to a consensus element located near the 3′ end of an intron also referred to as the “intron-exon boundary”. In one embodiment, an acceptor splice site comprises nucleotides of an intron-exon boundary comprising at least two nucleotides from the 3′ end of an intron and at least one nucleotide of the 5′ end of an exon.

As used herein, the term “acceptor essential splice site” refers to the last two nucleotides of the intron, denoted as positions A⁻² (second to last nucleotide of the intron) and A⁻¹ (last nucleotide of the intron). The skilled person will be familiar that the essential acceptor splice site is comprised of AG (adenine, guanine) nucleotides at the second last and last nucleotides of the intron, respectively, for ˜99% of human introns.

As used herein, the term “acceptor splice site sequence” refers to nucleotides comprised in an acceptor splice site. The skilled person will be familiar that the acceptor splice site sequence encompasses the branchpoint, the polypyrimidine tract and the acceptor essential splice site. In certain embodiments, an acceptor splice site sequence comprises 6 to 60 nucleotides of an acceptor splice site. In one embodiment, an acceptor splice site sequence comprises 6, 7, 8, or 9 consecutive nucleotides of an acceptor splice site. In certain embodiments, an acceptor splice site sequence comprises 9 consecutive nucleotides of an acceptor splice site.

As used herein, the term “cryptic donor splice site sequence” refers to a cryptic donor splice site sequence that is defined by any GT (or GC) that may constitute the consensus nucleotides of a donor essential splice site, wherein the cryptic donor splice site is not positioned correctly at the exon-intron junction. The skilled person will be familiar that abnormal splicing due to use of cryptic donor splice sites can occur in subjects with variants affecting the authentic reference donor splice site. The skilled person will also be familiar that abnormal splicing due to use of cryptic donor splice sites can occur in subjects with variants affecting (e.g. strengthening) cryptic donor splice sites. In certain embodiments, a cryptic donor splice site sequence comprises 4, 5, 6, 7, 8, 9, 10, 11, 12 or up to 15 consecutive nucleotides of a cryptic donor splice site. In certain embodiments, a cryptic donor splice site sequence consists of 12 nucleotides comprised of four overlapping sequences of nine consecutive nucleotides, corresponding to nucleotide positions E⁻⁴ to D⁺⁵, E⁻³ to D⁺⁶, E⁻² to D⁺⁷ and E⁻¹ to D⁺⁸, wherein the GT (or GC) represent the nucleotides comprising the essential splice site at positions D⁺¹ and D⁺² of the cryptic donor splice site;

As used herein the term “sample splice site” refers to a sample from the genome of a subject. The skilled person will be familiar with sequencing of the genome of a subject, including but not limited to a human adult, juvenile, infant, foetus, embryo, or gamete. A sample splice site may comprise a splice site comprising a splice site sequence obtained from the genome of a subject. It will be understood that a single gene may comprise multiple splice sites. It will be understood that a sample splice site may be derived from an identified region of an identified gene. In one embodiment, a sample splice site may be obtained from whole genome sequencing. In one embodiment, a sample splice site may be obtained from whole exome sequencing. In one embodiment, a sample splice site may be obtained from sequencing a panel of genes. In one embodiment, a sample splice site may be obtained from sequencing a single gene. Exemplary sample splice sites, include, but are not limited to, a donor splice site, a branch site, and an acceptor splice site.

As used herein, the term “subject”, includes, but is not limited to, a human suspected of suffering from or carrying a genetic disorder (autosomal dominant, autosomal recessive, X-linked dominant, X-linked recessive, Y-linked, mitochondrial, or somatic), a human at risk of cancer, or a human suspected of having an abnormal splice site.

As used herein, the term “sample splice site sequence” refers to nucleotides comprised in a sample splice site. A sample splice site sequence may comprise one or more regions of consecutive nucleotides of a sample splice site. In certain embodiments, a sample splice site sequence may comprise one or more regions of consecutive nucleotides with one or more groups consisting of a single nucleotide. In one embodiment, a sample splice site sequence comprises 4 to 12 nucleotides of a sample splice site. In one embodiment, a sample splice site sequence comprises 4, 5, 6, 7, 8, 9, 10, 11, or 12 consecutive nucleotides of a sample splice site. In certain embodiments, the sample splice site sequence comprises 4 to 15 nucleotides of a donor splice site. In certain embodiments, the sample splice site sequence comprises 4, 5, 6, 7, 8, 9, 10, 11, 12, 13, 14 or up to 15 consecutive nucleotides of a donor splice site. In certain embodiments, the sample splice site sequence comprises 30 or more nucleotides of a donor splice site. In certain embodiments, the sample splice site sequence comprises 30 or more consecutive nucleotides of a donor splice site. In certain embodiments, a sample splice site sequence comprises 9 consecutive nucleotides of a sample splice site. In one embodiment, a sample splice site sequence comprises nucleotides comprised in a donor splice site, a branch site, or an acceptor site. In certain embodiments, a sample splice site sequence comprises 4 to 12 nucleotides comprised in a donor splice site. In certain embodiments, a sample splice site sequence comprises 4, 5, 6, 7, 8, 9, 10, 11, or 12 consecutive nucleotides of a donor splice site. In certain embodiments, a sample splice site sequence comprises 8, 9, or 10 consecutive nucleotides of a donor splice site. In certain embodiments, a sample splice site sequence comprises 9 consecutive nucleotides of a donor splice site. In certain embodiments, the sample splice site sequence comprises 4 to 15 nucleotides of a donor splice site. In certain embodiments, the sample splice site sequence comprises 4, 5, 6, 7, 8, 9, 10, 11, 12, 13, 14 or up to 15 consecutive nucleotides of a donor splice site. In certain embodiments, the sample splice site sequence comprises 30 or more nucleotides of a donor splice site. In certain embodiments, the sample splice site sequence comprises 30 or more consecutive nucleotides of a donor splice site.

In certain embodiments, more than one sample splice site sequence(s) from a sample splice site are analysed in determining a risk of abnormal splicing of a sample splice site, wherein the sample splice site sequences are each comprised in the same sample splice site. The terms “non-identical” or “not identical” may be used with reference to two or more sample splice site sequences that are obtained from different regions of the same sample splice site and refer to the respective nucleotide positions of the sample splice site. For example, the consecutive nucleotide sequences of E⁻⁵ to D⁺⁴ and E⁻⁴ to D⁺⁵ of a sample donor splice site are non-identical or not identical nucleotide positions of a sample donor splice site sequence, the consecutive nucleotide sequences of E⁻⁵ to D⁺⁴, E⁻⁴ to D⁺⁵, and E⁻³ to D⁺⁶ of a sample donor splice site are non-identical or not identical nucleotide positions of a sample donor splice site sequence, and so on. In other words, non-identical or not identical refers to the sample splice site sequence as a whole, considering each nucleotide comprised in each sample splice site sequence. The term “overlapping” may be used with reference to two or more sample splice site sequences obtained from different regions of the same sample splice site and refers to sample splice site sequences comprising non-identical or not identical nucleotide positions, wherein at least one nucleotide of each of the two or more sample splice site sequences corresponds to the same nucleotide position from the sample splice site. For example, the consecutive nucleotide sequences of E⁻⁵ to D⁺⁴ and E⁻⁴ to D⁺⁵ of a sample donor splice site are non-identical or not identical nucleotide positions of a sample donor splice site sequence and also comprise overlapping nucleotide positions of the sample donor splice site sequence. Likewise, each of the consecutive nucleotide sequences of E⁻⁵ to D⁺⁴, E⁻⁴ to D⁺⁵, and E⁻³ to D⁺⁶ of a sample donor splice site are non-identical or not identical nucleotide positions of a sample donor splice site sequence and also comprise overlapping nucleotide position of the sample donor splice site sequence. In certain embodiments, comprising two or more sample splice site sequences from the same sample splice site, each sample splice site sequence may be envisioned as derived from a window sliding along a sample splice site. Various embodiments of sample splice site sequences derived from the same sample splice site considering a sliding window are depicted in Table 1 (below). In certain embodiments comprising two or more sample splice site sequences from the same sample splice site, each sample splice site sequence comprises a different number of nucleotides. In certain embodiments comprising two or more sample splice site sequences from the same sample splice site, each sample splice site sequence comprises the same number of nucleotides. In certain embodiments, a sliding window comprises 9 consecutive nucleotides along a sample splice site. In certain embodiments, the sample splice site sequence corresponds to nucleotide position E⁻⁵ to D⁺⁴, E⁻⁴ to D⁺⁵, E⁻³ to D⁺⁶, E⁻² to D⁺⁷, E⁻¹ to D⁺⁸, or D⁺¹ to D⁺⁹ of a donor splice site. In certain embodiments, the sample splice site sequence corresponds to nucleotide position E⁻⁴ to D⁺⁵, E⁻³ to D⁺⁶, E⁻² to D⁺⁷ and E⁻¹ to D⁺⁸ of a donor splice site. In certain embodiments, the method comprises one or more sample splice site sequence(s) from a sample splice site wherein the one or more sample splice site sequence(s) corresponds to one or more of the nucleotide positions E⁻⁵ to D⁺⁴, E⁻⁴ to D⁺⁵, E⁻³ to D⁺⁶, E⁻² to D⁺⁷, E⁻¹ to D⁺⁸, or D⁺¹ to D⁺⁹ of a donor splice site. In certain embodiments, the method comprises one or more sample splice site sequence(s) from a sample splice site wherein the one or more sample splice site sequence(s) corresponds to one or more of the nucleotide positions E⁻⁴ to D⁺⁵, E⁻³ to D⁺⁶, E⁻² to D⁺⁷ and E⁻¹ to D⁺⁸ of a donor splice site. Four exemplary embodiments relating to embodiments comprising at least six sample donor splice site sequences from a sample donor splice site are depicted below in Table 1 wherein the nucleotides of a sample donor splice site are indicated as nucleotide positions E⁻⁵ to D⁺⁹ and an “x” indicates that that nucleotide is included in a sample donor splice site sequence and wherein the left most column in the table is the arbitrary number assigned the sample splice site sequence (1 is the first sample splice site sequence, 2 is the second splice site sequence, and so on).

TABLE 1

| | E⁻⁵ | E⁻⁴ | E⁻³ | E⁻² | E⁻¹ | D⁺¹ | D⁺² | D⁺³ | D⁺⁴ | D⁺⁵ | D⁺⁶ | D⁺⁷ | D⁺⁸ | D⁺⁹ |
|---|---|---|---|---|---|---|---|---|---|---|---|---|---|---|
| 1 | x | x | x | x | x | x | x | x | x | | | | | |
| 2 | | x | x | x | x | x | x | x | x | x | | | | |
| 3 | | | x | x | x | x | x | x | x | x | x | | | |
| 4 | | | | x | x | x | x | x | x | x | x | x | | |
| 5 | | | | | x | x | x | x | x | x | x | x | x | |
| 6 | | | | | | x | x | x | x | x | x | x | x | x |
| 1 | x | x | x | x | x | x | x | x | x | | | | | |
| 2 | x | x | x | x | x | x | x | x | x | x | | | | |
| 3 | x | x | x | x | x | x | x | x | x | x | x | | | |
| 4 | x | x | x | x | x | x | x | x | x | x | x | x | | |
| 5 | x | x | x | x | x | x | x | x | x | x | x | x | x | |
| 6 | x | x | x | x | x | x | x | x | x | x | x | x | x | x |
| 1 | | | | | | x | x | x | x | x | x | x | x | x |
| 2 | | | | | x | x | x | x | x | x | x | x | x | x |
| 3 | | | | x | x | x | x | x | x | x | x | x | x | x |
| 4 | | | x | x | x | x | x | x | x | x | x | x | x | x |
| 5 | | x | x | x | x | x | x | x | x | x | x | x | x | x |
| 6 | x | x | x | x | x | x | x | x | x | x | x | x | x | x |
| 1 | | | | x | x | x | x | x | x | | | | | |
| 2 | | | x | x | x | x | x | x | x | x | | | | |
| 3 | | x | x | x | x | x | x | x | x | x | x | | | |
| 4 | x | x | x | x | x | x | x | x | x | x | x | x | | |
| 5 | x | x | x | x | x | x | x | x | x | x | x | x | x | |
| 6 | x | x | x | x | x | x | x | x | x | x | x | x | x | x |

As used herein, the term “reference splice site sequence” refers to a splice site sequence from a sequenced human genome, referred to herein as a reference human genome sequence. Exemplary reference human genome sequences include, but are not limited to, the “Genome Reference Consortium Build 37” also referred to as “hg19” (<https://www.ncbi.nlm.nih.gov/assembly/GCF_000001405.13>), Genome Reference Consortium Human Build 38 patch release 12 (GRCh38.p12) (<https://www.ncbi.nlm.nih.gov/assembly/GCF_000001405.38>), or any sequenced human genome from an individual or individuals not exhibiting or carrying a genetic disorder. In one embodiment, a reference human genome is the human genome sequence of the “Genome Reference Consortium Build 37” also referred to as “hg19” (<https://www.ncbi.nlm.nih.goviassembly/GCF_000001405.13>). In one embodiment, a reference human genome is the human genome sequence of the Genome Reference Consortium Human Build 38 patch release 12 (GRCh38.p12) (<https://www.ncbi.nlm.nih.gov/assembly/GCF_000001405.38>). In one embodiment, a reference human genome is a combination of the human genome sequence of the “Genome Reference Consortium Build 37” also referred to as “hg19” (<https://www.ncbi.nlm.nih.goviassembly/GCF_000001405.13>) and the human genome sequence of the Genome Reference Consortium Human Build 38 patch release 12 (GRCh38.p12) (<https://www.ncbi.nlm.nih.gov/assembly/GCF_000001405.38>).

As used herein, the term “corresponding” with regard to the terms “corresponding gene”, “same corresponding region of a gene”, “corresponding reference splice site”, and “corresponding reference splice site sequence”, and variations thereof, are used to denote that a sample splice site and a corresponding reference splice site are derived from the same region of the same gene, wherein the sample splice site comprises nucleotide sequences obtained from genomic sequencing of a subject and the corresponding reference splice site comprises nucleotides from a reference human genome sequence. For example, when the sample splice site comprises nucleotides E⁻⁵ to D⁺⁵ of the exon-intron boundary of exon 5 of gene X from a subject, the reference splice site comprises nucleotides E⁻⁵ to D⁺⁵ of the exon-intron boundary of exon 5 of gene X from a reference human genome sequence. Likewise, for example, a sample splice site sequence of nucleotides D⁺¹ to D⁺⁵ of the exon-intron boundary of exon 5 of gene X from a subject will have a reference splice site of nucleotides D⁺¹ to D⁺⁵ of the exon-intron boundary of exon 5 of gene X from a reference human genome sequence.

As used herein, the term “Native Intron Frequency” refers to frequency a particular nucleotide sequence appears in a splice site in a reference human genome sequence. One measure of Native Intron Frequency is the number of times a particular nucleotide sequence appears in a splice site in a reference human genome sequence, which may be represented by NIFᵥₐᵣ or NIF (count). In certain embodiments, a measure of Native Intron Frequency of a reference splice site sequence (NIF_{ref}) refers to the number of times the nucleotide sequence of the reference splice site sequence appears in splice sites in a reference human genome sequence; a measure of Native Intron Frequency of the sample splice site sequence (NIFᵥₐᵣ) refers to the number of times the nucleotide sequence of the sample splice site sequence appears in a splice site in a reference human genome sequence; a NIF equal to 0 (zero) (NIF=0) means that the nucleotide sequence does not appear in any splice site in a reference human genome sequence; a NIF equal to one (NIF=1) means that the nucleotide sequence appears in one splice site in a reference human genome sequence; an NIF equal to two (NIF=2) means that the nucleotide sequence appears in two splice sites in a reference human genome sequence, wherein each of the two splice sites is a unique splice site in the reference human genome; an NIF equal to three (NIF=3) means that the nucleotide sequence appears in three splice sites in a reference human genome sequence, wherein each of the three splice sites is a unique splice site in the reference human genome; and so on. “Unique” as used in this context refers to each splice sequence appearing in a different splice site in one gene or two different genes. For example, a sample donor splice site sequence having an NIF=2 means that the nucleotide sequence of the sample donor splice site sequence appears in two different donor splice sites (different exon-intron boundaries), wherein the two different splice sites may be from two splice sites within the same gene or two splice sites from two different genes. The symbol NIFᵥₐᵣ₋ₓ, where “x” is a whole number integer (1, 2, 3, 4, 5, and so on) refers to the measure of Native Intron Frequency determination for a sample splice site where more than one sample splice site sequence from the same sample splice site is analysed. For example, where two sample splice site sequences are analysed from the same splice site, an NIFᵥₐᵣ for the first sample splice site sequence may be referred to as NIFᵥₐᵣ₋₁ and an NIFᵥₐᵣ for the second sample splice site sequence may be referred to as NIFᵥₐᵣ₋₂; and so on. The corresponding two NIF_{ref} for each reference splice site sequence, one for the first splice site sequence and two for the second splice site sequence, may be referred to as NIF_{ref-1} and NIF_{ref-2}, respectively; and so on.

As used herein, the term “abnormal splice site” refers to the characterization of splice site as a genetic variant of the corresponding splice site of a reference human genome sequence, wherein the genetic variant exhibits aberrant splicing. Aberrant splicing includes, but is not limited to, reduced splicing, non-splicing, exon-skipping, intron retention, and the like. Aberrant splicing associated with an abnormal splice site may be causative of a pathogenic phenotype. An abnormal splice site may be further characterized as a pathogenic splice site wherein aberrant splicing associated with an abnormal splice site is causative of a pathogenic phenotype. An abnormal splice site may be characterized with a risk of abnormal splicing. In one embodiment, a risk of abnormal splicing is characterized by a value from 0 to 1, wherein the risk of abnormal splicing increases as the value approaches 1.

As used herein, the term “abnormal splice site sequence” refers to a splice site sequence that comprises a different nucleotide sequence when compared with the splice site sequence in the corresponding region of a gene in a reference human genome sequence. An abnormal splice site sequence may be further characterized as a pathogenic splice site sequence, wherein aberrant splicing associated with the abnormal splice site sequence is causative of a pathogenic phenotype. A genetic variant may comprise an abnormal splice site comprising an abnormal splice site sequence.

As used herein, the term “benign variant splice site” refers to a splice site sequence that comprises a different nucleotide sequence when compared with the splice site sequence in the corresponding region of a gene in a reference human genome sequence, and does not result in aberrant splicing.

As used herein, the term “clinical classification” refers to the classification assigned to a splice site. Clinical classification for a splice site may be determined from any available source wherein a genetic variant is assigned a clinical classification. Exemplary sources of variant splice sites with clinical classifications include, but are not limited to, ClinVar (<https://www.ncbi.nlm.nih.gov/clinvar/>) and the Human Gene Mutation Database (HGMD) (<http://www.hgmd.cf.ac.uk/ac/index.php>). The skilled person will be familiar with clinical classifications assigned to variant genes, variant splice sites, and variant splice site sequences. See, e.g., Richards et al, Genetics in Medicine (2015) 17(5): 405-424. Clinical classifications in ClinVar include pathogenic, likely pathogenic, benign, and likely benign among others. Entries included in the HMGD may be identified as gene lesions responsible for human inherited diseases and as such are classified as pathogenic. A region of a splice site, for example 4, 5, 6, 7, 8, 9, 10, 11, or 12 nucleotides of a splice site sequence, may appear in more than one splice site, with each appearance represents a genetic variant and each appearance may be assigned a clinical classification. A region of a splice site, for example 4, 5, 6, 7, 8, 9, 10, 11, 12 or up to 15 nucleotides of a splice site sequence, may appear in more than one splice site, with each appearance represents a genetic variant and each appearance may be assigned a clinical classification. A region of a splice site, for example up to 15 nucleotides or more of a splice site sequence, may appear in more than one splice site, with each appearance represents a genetic variant and each appearance may be assigned a clinical classification. A region of a splice site, for example up to 30 nucleotides or more of a splice site sequence, may appear in more than one splice site, with each appearance represents a genetic variant and each appearance may be assigned a clinical classification. A clinical classification associated with a nucleotide sequence of a splice site sequence (eg a sample splice site sequence or a reference splice site sequence) includes any clinical classification assigned to the nucleotide sequence in any splice site in any gene. A clinical classification of a splice site as pathogenic or likely pathogenic may be interpreted as an abnormal splice site (also referred to herein a pathogenic splice site). A clinical classification of a splice site as benign or likely benign may be interpreted as a benign variant splice site.

As used herein, the term “Percentile (NIF)” (alternatively herein referred to as “NIF percentile”) refers to the percentile within the percentile distribution of the frequency of a splice site sequence in a reference human genome sequence. A NIFᵥₐᵣ of 0 (zero) is assigned a 0th Percentile (NIFᵥₐᵣ). For example, a NIFᵥₐᵣ within the 2^{nd} Percentile indicates that, for splice site sequences comprised in a reference human genome sequence, <2% of splice site sequences have a NIF falling within this range; an exemplary NIF_{ref} of 653 lies within the 85^{th} percentile among a frequency distribution of splice site sequences in a reference human genome; and so on.

As used herein, median percentile NIF is calculated as median (NIFᵥₐᵣ₋₁ percentile; NIFᵥₐᵣ₋₂ percentile; percentile of NIFᵥₐᵣ₋₃ percentile; NIFᵥₐᵣ₋₄ percentile). For example, a hypothetical site with percentile NIFᵥₐᵣ₋₁=0.2499, percentile NIFᵥₐᵣ₋₂=0.5904, percentile NIFᵥₐᵣ₋₃=0.7172, percentile NIFᵥₐᵣ₋₄=0.9065 has a median percentile NIFᵥₐᵣ₋ₓ of 0.6538. This may also be represented generically by median (NIF_{ref-1}; NIF_{ref-2}; NIF_{ref-3}; NIF_{ref-4}).

As used herein, the Percentile value for median NIF is determined through calculation of the cumulative frequency distributions of median NIF_{ref-x} for all donor splice sites in the reference human genome (180,000 donor splice sites). For example, a donor splice site of 12 nucleotides with a median NIF_{ref1-4} of 1 lies within the first percentile of a frequency distribution of median NIF_{ref1-4} among all donor splice sites in the reference human genome. In a second example, a donor splice site with a median NIF_{ref1-4} of 327 lies within the fiftieth percentile of a frequency distribution of median NIF_{ref1-4} among all donor splice sites in the reference human genome

As used herein, the term “NIF-shift” refers to a measure of the relative change in NIF for a given splice site sequence with respect to a corresponding reference human genome sequence. In one embodiment, NIF-shift may be determined by comparing a measure of NIF for a given splice site sequence with a measure of NIF for the corresponding reference splice site sequence. In one embodiment, NIF-shift of a sample splice site sequence may be determined by comparing a measure of NIF of a sample splice site sequence (NIFᵥₐᵣ₋ₓ) with a measure of NIF of the corresponding reference splice site sequence (NIF_{ref-x}). In one embodiment, NIF-shift is determined by a comparison of Percentile (NIFᵥₐᵣ₋ₓ) with the corresponding Percentile (NIF_{ref-x}). In a second embodiment, median NIF-shift of a sample splice site sequence may be determined by comparing a measure of median NIF of a sample splice site sequences (median NIFᵥₐᵣ₋ₓ) with a measure of median NIF of the corresponding reference splice site sequences (median NIF_{ref-x}). In a related embodiment, percentile median NIF-shift of a sample splice site sequence may be determined by comparison of Percentile (median NIFᵥₐᵣ₋ₓ) with the corresponding Percentile (median NIF_{ref-x}). In certain embodiments, comparing, e.g. NIFᵥₐᵣ₋ₓ with corresponding NIF_{ref-x} or Percentile (NIFᵥₐᵣ₋ₓ) with corresponding Percentile (NIF_{ref-x}), to determine NIF-shift comprises a ratiometric analysis, e.g. NIFᵥₐᵣ₋ₓ/NIF_{ref-x}, Percentile (NIFᵥₐᵣ₋ₓ)/Percentile (NIF_{ref-x}), median (NIFᵥₐᵣ₋ₓ)/median (NIF_{ref-x}), Percentile (median NIFᵥₐᵣ₋ₓ)/Percentile (median NIF_{ref-x}), mean (NIFᵥₐᵣ₋ₓ)/mean (NIF_{ref-x}), Percentile (meanNIFᵥₐᵣ₋ₓ)/Percentile (mean NIF_{ref-x}). In certain embodiments, comparing, e.g. NIFᵥₐᵣ₋ₓ with corresponding NIF_{ref-x} or Percentile (NIFᵥₐᵣ₋ₓ) with corresponding Percentile (NIF_{ref-x}), to determine NIF-shift comprises subtracting, e.g. subtracting NIFᵥₐᵣ₋ₓ from NIF_{ref-x} or subtracting Percentile (NIFᵥₐᵣ₋ₓ) from Percentile (NIF_{ref-x}).

As used herein, the term “same NIF-shift” refers to two or more splice site sequences having about the same “NIF-shift” or the same “NIF-shift”. In certain embodiments, the term “same median NIF-shift” refers to two or more splice site sequences having about the same “median NIF-shift” or the same “median NIF-shift”. In related embodiments, the term “same mean NIF-shift” refers to two or more splice site sequences having about the same “mean NIF-shift” or the same “mean NIF-shift”.

As used herein, the term “similar NIF-shift variant” refers to a splice site sequence having a relative change (or shift) in NIF (or Percentile NIF), median NIF (or Percentile median NIF) or mean NIF (or Percentile mean NIF) with respect to a corresponding reference human genome sequence (referred to herein as a NIF-shift), which is similar to a relative change (or shift) in NIF with respect to a corresponding reference human genome sequence for another splice site sequence. Two or more splice site sequences are considered “similar NIF-shift variants”, when two or more splice site sequences have the same relative change (or shift) in NIF or fall within the same range of values around a NIF-shift of a sample splice site sequence. In certain embodiments, a range of values around a NIF-shift is ±about 2%, ±about 2.5%, ±about 5%, or ±about 10%. For example, for sample splice site sequence with median NIFᵥₐᵣ₋ₓ of 0 and a corresponding median NIF_{ref-x} of 653, similar median NIF-shift variants can have a NIFᵥₐᵣ of 0 and a corresponding NIF_{ref} of from 472-903. For a sample splice site sequence and its corresponding reference splice site sequence having Percentile (median NIFᵥₐᵣ₋ₓ)=0 and Percentile (median NIF_{ref-x})=0.85 (85^{th} percentile), a similar NIF-shift variant(s) would include, but would not be limited to, a splice site sequence and its corresponding reference splice site sequence having Percentile median NIFᵥₐᵣ₋ₓ=0 and a range of values around Percentile median NIF_{ref}=0.85. In certain embodiments, a range of median NIF-shift values may be calculated, wherein a lower bound and an upper bound may be determined for each median NIFᵥₐᵣ₋ₓ and corresponding median NIF_{ref-x} or Percentile (median NIFᵥₐᵣ₋ₓ) and corresponding Percentile (median NIF_{ref-x}), or calculated from a median NIF-shift, eg, ratiometric or subtraction of median NIF-shift, to calculate a range of median NIF-shift. For example, a ±about 2% NIF-shift range could be calculated considering ±about 2% NIFᵥₐᵣ₋ₓ and ±about 2% NIF_{ref-x}; and a similar NIF-shift variant will have a have a NIFᵥₐᵣ and NIF_{ref} with the calculated ranges. In certain embodiments, the range of NIF-shift may be determined by considering exponential upper and lower bounds. For example, a lower bound (e^{((log(NIFvar))*(1-NIF_shift percentage))}) and an upper bound (e^{((log(NIFvar))*(1+NIF_shift percentage))}) for NIFᵥₐᵣ and a lower bound (e^{((log(NIFref))*(1-NIF_shift_percentage))}) and an upper bound (e^{((log(NIFref))*(1+NIF_shift percentage))}) for NIF_{ref} may be used to calculate a range of NIF-shift for identifying similar NIF-shift variants. In this context, suitable NIF-shift percentages include about 2%, about 2.5%, about 5%, and about 10%.

As used herein, the term “Clinical Splice Predictor (CSP) reference database” refers to a database of variant splice sites with clinical classifications, for example abnormal splice site or benign variant splice site. Clinical classification for a splice site may be determined from any available source wherein a genetic variant is assigned a clinical classification. Exemplary sources of variant splice sites with clinical classifications include, but are not limited to, ClinVar (<https://www.ncbi.nlm.nih.gov/clinvar/>) and the Human Gene Mutation Database (HGMD) (<http://www.hgmd.cf.ac.uk/ac/index.php>). The skilled person will be familiar with clinical classifications assigned to variant genes, variant splice sites, and variant splice site sequences. See, eg, Richards et al, Genetics in Medicine (2015) 17(5): 405-424. Clinical classifications in ClinVar include pathogenic, likely pathogenic, benign, and likely benign among others. Entries included in the HMGD may be identified as genes lesions responsible for human inherited diseases and as such are classified as pathogenic. A clinical classification of a variant splice site as pathogenic or likely pathogenic may be interpreted as an abnormal splice site. A clinical classification of a variant splice site as benign or likely benign may be interpreted as a benign variant splice site. In one embodiment, a CSP reference database includes variant splice sites clinically classified as an abnormal splice site or a benign variant splice site. In certain embodiments, a CSP reference database comprises variants, wherein a variant splice site clinically classified as “pathogenic” or “likely pathogenic” is assigned as an “abnormal splice variants” and wherein a variant splice site clinically classified as “benign” or “likely benign” is assigned as a “benign variant splice site”. A CSP reference database may comprise variants affecting only a donor splice site, including exonic variants that are are non-code changing variants (synonymous exonic variants).

As used herein, the term “genetic disorder” includes a disorder that reflects inheritance of a single causative gene. Exemplary sources of genes underlying a genetic disorder include, but are not limited to, Online Genetic Inheritance in Man (OMIM, found at <https://www.omim.org/>. See Appendix A for a list of OMIM genes.

### BRIEF DESCRIPTION OF THE FIGURES

Embodiments of the invention will now be described, by way of example only, with reference to the accompanying drawings as follows.

FIG. 1: Embodiment of a Clinical Splice Predictor (CSP) Reference Database. A) Workflow used to amalgamate variant splice sites with clinical classifications from Clinvar and HGMD, filtering of variants to include only: single nucleotide polymorphisms (SNPs), variants with clinical classification as benign (for ClinVar variants; benign or likely benign) or pathogenic (for ClinVar variants; pathogenic or likely pathogenic), synonymous exonic variants. B) Workflow describing how the nucleotide sequence for sample and reference splice site is extracted from a human reference genome and appended with Native Intron Frequency metrics.

FIG. 2: Workflow describing determination of Native Intron Frequency (NIF) in relation to embodiments related to embodiment 2. A. Depicting predictive model. B. Embodiment related to embodiment 2 comprising determining NIFᵥₐᵣ and NIF_{ref}. C. Embodiment related to embodiment 2 comprising determining Percentile (NIFᵥₐᵣ) and Percentile (NIF_{ref}).

FIG. 3: Workflow describing determination of Previous Classification Factor determination. A. Depicting predictive model. B. Embodiment related to embodiment 3 comprising determining clinical classifications for a first reference splice site sequence and a corresponding first reference splice site sequence, the latter of which is optional in related embodiments.

FIG. 4: A. Workflow describing determination of Same NIF-Shift. B. Workflow describing determination of Similar NIF-Shift.

FIG. 5: Receiver Operator Characteristic curves. Clinical Splice Predictor v2. A) Clinical Splice Predictor (v2) method (CSP, magenta line) shows higher sensitivity and specificity than each of the predictive splicing methods run by Alamut®Visual biosoftware. ROC curves shown source 2,255 test variants from CSP Reference Database V2, for which predictions were offered by all five predictive methods within Alamut®Visual biosoftware. CSP Reference Database V2 is comprised of 4745 ClinVar sample splice site variants (positions D⁺¹ to D⁺⁶ of a donor splice site) with 30% variants (randomised) used for machine learning and 70% used as test variants. AUC: Area under curve. B) Diagnostic efficacy for extended splice donor variants (dashed lines; positions D⁺³ to D⁺⁶ of a donor splice site. NOTE: 1. Clinical Splice Predictor (v2) operates using five, 9 nucleotide windows, spanning E⁻⁵ (fifth to last base of the exon) to D⁺⁸ (eighth base into the intron). 2. Clinical Splice Predictor (v2) weights two binary inputs by logistic regression; Native intron frequency (NIF) and Previous Classifications in ClinVar as benign (benign variant splice site) or pathogenic (abnormal splice site). 3. Sensitivity is a measure of True Positive detection rate; i.e. for 100 pathogenic variants, how many are correctly identified as pathogenic. 4. Specificity is a measure of False Positive detection rate; i.e. for 100 benign variants, how many are incorrectly identified as pathogenic.

FIG. 6: Receiver Operator Characteristic curves of source binary inputs for Clinical Splice Predictor v2. A) Receiver Operator Characteristic (ROC) curves for extracted ClinVar donor splice site variants D⁺¹ to D⁺⁶ (n=4745), with 30% variants (randomised) used for machine learning and 70% used as test variants. NIF E3˜D6: Native Intron Frequency analysed as a measure. Analysis of one window of nine nucleotides (nt) spanning E⁻³ to D⁺⁶. Percentile (NIF) E3˜D6: Native Intron Frequency analysed as a percentile calculation. Analysis of one window of nine nucleotides spanning E⁻³ to D⁺⁶. Percentile (NIF) 9 nt sliding E5˜D8: Weights NIF percentile information from all windows the variant lies within (five, 9 nt sliding windows are examined, spanning E⁻⁵ to D⁺⁸). Previous Classifications, E3˜D6. Previous clinical classifications of the variant donor splice site spanning E⁻³ to D⁺⁶. Similar NIF-Shift variants. Previous clinical classifications of variant donor splice sites that show the same shift in NIF between the reference and variant donor splice site, independent of specific nucleotide sequence. Prev.Class^{fns} & % NIF sliding E5˜D8: Combines Previous Classifications (E⁻³ to D⁺⁶ window) and Percentile (NIF) using five sliding windows of 9 nucleotides spanning E⁻⁵ to D⁺⁸.

FIGS. 7A-7D: Clinical Splice Predictor V3: Histograms showing the effectiveness of each binary input to discriminate a benign variant splice site from abnormal splice site (labelled as “pathogenic”). CSP Reference database V3 sources 13,484 donor splice site variants extracted from ClinVar and HGMD from E⁻⁴ to D⁺⁸ (Pathogenic 10,210; Benign 3,274). A) E⁻⁴ to D⁺⁵ window of nine consecutive nucleotides of the donor splice site sequence. B) E⁻³ to D⁺⁶ window of nine consecutive nucleotides of the donor splice site sequence. C) E⁻² to D⁺⁷ window of nine consecutive nucleotides of the donor splice site sequence. D) E⁻¹ to D⁺⁷ window of nine consecutive nucleotides of the donor splice site sequence. i) Native Intron Frequency (NIF): Left: NIF for the reference splice site sequence (NIF_{ref}) for benign (benign variant splice site) (blue) and pathogenic (abnormal splice site) (red) variants. Right: NIF for the variant donor splice site (NIFᵥₐᵣ) for benign (benign variant splice site) (blue) and pathogenic (abnormal splice site) (red) variants. ii) Previous Classifications. Left: Frequency a given pathogenic 9 nucleotide donor splice site sequence (abnormal splice site sequence) has been classified previously as pathogenic (abnormal splice site) or benign (benign variant splice site). Right: Frequency a given benign 9 nucleotide donor splice site sequence (benign variant splice site) has been classified previously as pathogenic (abnormal splice site) or benign (benign variant splice site). ii) Similar NIF-Shift variants. The ratio of pathogenic (abnormal splice site)/benign (benign variant splice site) reports among variant donor splice sites that show a similar shift in NIF between the reference and variant donor splice site sequences. For each variant, similar NIF-shift variants are defined as those that fall within +/−5^{th} percentile on a Log₁₀ frequency distribution of NIF_{ref}, which are similarly transformed to +/−5^{th} percentile on a Log₁₀ frequency distribution of NIFᵥₐᵣ. Log₁₀ frequency distribution enables the greatest granularity in the important diagnostic range between NIF=0 and NIF=10.

FIG. 8: CSPv3 Test Run of ˜1,000 ‘likely benign’ donor splice site variants. A sample cohort greatly enriched for ‘benign variant splice sites’ were derived from gnomAD using the following filters: 1. Single nucleotide polymorphisms affecting positions E⁻⁴ to D⁺⁸ of a donor splice site. 2. Variants not already existing within the CSP Reference database V3. 3. Only synonymous exonic variants. 3. Variants with five or more homozygous individuals. 4. Variants in genes with; i) High loss-of-function constraint pLi=>0.9), ii) Genes where recessive null alleles in mouse models is associated with pre-weaning lethality (see Appendix B), iii) Genes where a dominant or recessive null allele(s) is associated with human lethal syndromes (perinatal or neonatal death <3 months of age, see Appendix C). A) Native Intron Frequency (NIF). Left: NIF of the reference donor splice site (NIF_{ref}) for CSPv3 pathogenic (abnormal splice site) (black), benign (benign variant splice site) (light grey) and gnomAD (dark grey) variants. Right: NIF of the variant donor splice site (NIFᵥₐᵣ) for CSPv3 pathogenic (abnormal splice site) (black), benign (benign variant splice site) (light grey) and gnomAD (dark grey) variants. B) Previous Classifications. Left: Frequency a given pathogenic splice site (abnormal splice site) has been classified previously as pathogenic, benign or benign-like (gnomAD). Right: Frequency a given benign variant splice site has been classified previously as pathogenic, benign or benign-like (gnomAD).

FIG. 9: Embodiment supporting the utility of NIF=0 for prediction of abnormal splice sites. Data sources CSP Reference database V3: 13,484 donor splice variants extracted from ClinVar and HGMD from E⁻⁴ to D⁺⁸ (Pathogenic 10,210; Benign 3,274). A) Variant splice sites with NIF of 0 are a strong biomarker of clinically classified pathogenic splice sites (abnormal splice sites). 65.0% of all pathogenic variants create a variant donor splice site where all four windows contain a combination of 9 consecutive nucleotides that do not exist at any donor splice site at an exon/intron boundary in the reference human genome sequence (hg19 build). In contrast, only 0.7% of benign variants have all four windows with NIF=0. B) Pie charts showing the relative percentage of variant splice sites with at least one 9 nucleotide window with NIF=0. On average, ˜75% pathogenic variants have at least one NIF=0, whereas only ˜2.5% benign variants have at least one NIF=0. C) Odds ratio analyses demonstrate NIF=0 is a potent biomarker of abnormal splicing. The odds that a sample splice site is a pathogenic splice site (abnormal splice site) increases incrementally with one or more windows with NIF=0. Variant sample splice sites with four windows with NIF=0 are 961 times more likely to be pathogenic than benign (compared to variant sample splice sites with no windows NIF=0). Whereas, genetic variants creating sample splice sites with a low NIF of 1-9, but not NIF=0, are only 9.4 times more likely to be pathogenic than benign. Conversely, variant sample splice sites that maintain or increase NIF (relative to the reference splice site) are 145 times more likely to benign than pathogenic. D) Receiver Operator Characteristic Curve NIF percentile: CSPv3. E) Receiver Operator Characteristic Curve NIF Count: CSPv3.

FIG. 10: Embodiment supporting predictive utility of Previous Classifications (PC). A) An example demonstrating how the same combination of nine nucleotides can be created by different variants affecting different positions of extended splice donor. B) Odds ratio analyses. Odds that a variant splice-site is pathogenic (i.e. induces abnormal splicing) increase by ˜200 fold when a variant splice site has at least one non-conflicting classification as pathogenic (P-only), or when pathogenic classifications outnumber benign classification (P>B) in any window. C) Odds ratio cross-validation was performed by ten, randomly sampled subsets of 1000 pathogenic variants compared with 1000 benign variants, extracted from the CSPv3 source database. Each sample of 1000 variants has varying ratios of benign versus pathogenic variants with at least one previous classification. Odds-ratios values listed below therefore represent the mean, plus or minus standard deviation, of ten random samples of 1000 variants. D) Graphical representation of Previous Classifications among random sample No. 1 (from FIG. 10B, above). The vast majority of benign variant splice sites (in windows of 9 consecutive nucleotides) have been classified previously only as benign (light grey bar, benign variants). Vice-versa, the vast majority of pathogenic splice sites (in windows of 9 consecutive nucleotides) have been classified previously only as pathogenic (black bar, pathogenic variants). D) Receiver operator characteristic curve: Previous Classifications Clinical Splice Predictor V3. NOTE: This ROC curve shows reduced sensitivity and specificity than shown in FIG. 6 with CSPv2, as CSPv2 factored every ClinVar submission for a given variant. For example, the specific variant ABCB4; NM_000443.3:c.2064+3A>T may have been reported by different submitters as pathogenic on thirteen occasions, and benign once. All fourteen submissions were weighted by CSPv2. In contrast, for CSPv3 to amalgamate ClinVar variants with HGMD variants, multiple ClinVar submissions were collapsed for a given variant to a single classification as benign, or pathogenic, based on the numerical excess of submissions in one clinical category.

FIG. 11: Odds ratio analyses demonstrate cumulative predictive power of combining native intron frequency and previous classifications. Odds that a variant splice-site is pathogenic increase substantially when NIF and Previous Classifications are combined. Odds ratio analyses were performed for ten, randomly sampled subsets of 1000 pathogenic variants compared with 1000 benign variants, extracted from the CSPv3 source database. Each sample of 1000 variants has varying ratios of benign versus pathogenic variants with previous classifications available. Odds-ratios values listed therefore represent the mean of ten random samples of 1000 variants.

FIG. 12: An exemplary embodiment of a method of identifying an abnormal splice site comprising generating a first, second, and third abnormal splicing factor.

FIGS. 13A-13B: A. Exemplification of a window of a sample splice site. B. Subset of sample splice site is exemplified.

FIG. 14: Examples of RNA Sequencing data confirming CSPv3 predictions in the Blinded Trial shown in Table 3. Sashimi plots depicting RNA sequencing of a subject. The coloured peaks represent RNA sequencing reads covering an exon. The connecting loops represent RNA reads bridging more than one exon and indicative of splicing from one exon to another. “Case 2”, “Case 10”, and so on, refers to cases described within Table 3. Red arrow(s): denote individual(s) carrying the variant at heterozygosity or homozygosity. Other RNA-sequencing traces in the screen shot are from disease controls; indicative of typical levels of normal splicing or abnormal splicing at a given exon-intron junction. Text boxes: Brief comments explaining strength of RNA sequencing read depth and consequences for pre-mRNA splicing observed to result from a genetic variant affecting the donor splice site.

FIG. 15: Plot representing cumulative frequency distribution of all human introns (GRCh37). X axis represents median NIFᵥₐᵣ₋ₓ; Y axis represents cumulative no. of introns. Vertical dotted lines represent the median percentile NIFᵥₐᵣ₋ₓ cutoffs.

FIG. 16: 5 plots representing Logistic regression performance summary (Receiver Operator Curve) for combination of binary inputs for Clinical Splice Predictor v7. The inputs can consist of Native Intron Frequency (NIF), Previous Classification Factor and Same NIF-Shift used independently or in combination.

FIG. 17: Embodiment supporting the utility of source binary inputs for Clinical Splice Predictor v7. Data sources the CSPv7 reference database of 14,875 variants affect 9,670 unique 5′ splice sites across 1984 clinically relevant OMIM genes. A) Native Intron Frequency and odds of mis-splicing. Data shown represents the net change in Percentile median Native Intron Frequency (median NIF, with net change calculated as Var/Ref) for pathogenic (red) or benign (blue) variants in the CSP database. Upper graph: Frequency distribution plot of the net change in Percentile median NIF relative to clinical classification as pathogenic or benign. Note: This graph only shows data for extended splice site variants with the CSPv7 database (˜5,000 variants). Essential splice site variants are omitted, as the vast majority create a net percentile change of zero (see source data presented in FIG. 8). Lower Graph: Odds a sample variant will be pathogenic or benign based on the net change in Percentile median NIF. Y-axis: odds ratio on a logarithmic scale. X-axis: Categories as defined by the net change in Percentile median NIF. Net change calculated as percentile (median NIFvar1-4)/percentile (median NIFref1-4). Data shown excludes D+1 and D+2 essential donor splice site variants in the CSPv7 reference database, as the overwhelming majority of essential donor splice site variants have median NIFvar1-4) in the zeroth percentile, rendering >7,000 variants on the y-axis and confounding interpretation of data for extended donor splice site variants, B) Previous Classification Factor binary and odds of mis-splicing. Note: Previous Classification Factor binary is termed Previous Clinical Variants (PCV)). PCV are clinical variants in the CSPv7 reference database that have resulted in the same combination of nine, consecutive nucleotides at the analogous position of the exon-intron junction as the sample variant. Variants classified as benign or likely benign are viewed collectively as benign. Variants classified as pathogenic or likely pathogenic are viewed collectively as pathogenic. Y-axis: odds ratio on a logarithmic scale. X-Axis: [1,2] corresponds to PCV at 1 or 2 genetic loci. (2,5] corresponds to PCV at 3-5 genetic loci. (5,10] corresponds to PCV at 6-10 genetic loci. (10,210] corresponds to PCV at 10-210 genetic loci. The three sections show the relative decrease in odds as PCVs with conflicting classifications occur. Data shown includes all donor splice site variants in the CSPv7 reference database. C) Similar NIF-Shift (SNS) binary and odds of mis-splicing. Upper graph: Frequency distribution plot of variants within the CSPv7 database and the corresponding percentage of pathogenic or benign SNS variants. For example, the extreme left hand side shows the number of CSPv7 variants with 100% of SNS variants classified as pathogenic, 99% of SNS variants classified as pathogenic, and so on as you move right, with the extreme right hand side showing number of CSPv7 variants with 100% of SNS variants classified as benign. Lower graph: The corresponding odds ratio supporting classification of a sample variant as pathogenic or benign, based on the percentage of pathogenic or benign SNS variants. Box bracket “[” depicts inclusive of value. Parenthesis “(” depicts exclusive of value. Similar NIF-shift variants calculated using upper and lower bounds of percentile (median NIFvar1-4 and percentile (median NIFref1-4). Data shown includes all donor splice site variants in the CSPv7 reference database.

FIG. 18: Source data informing Odds Ratio calculations for CSPv7. A) Represents odds of a variant being Pathogenic (i.e. splice altering) or Benign (i.e. non splice altering) based on Native Intron Frequency (NIF) binary. B) Represents odds of a variant being Pathogenic (i.e. splice altering) based on Previous Classification Factor binary. C) Represents odds of a variant being Pathogenic (i.e. splice altering) or Benign (i.e. non splice altering) based on Same NIF-Shift binary. Data sources the CSPv7 reference database of 14,875 variants affect 9,670 unique 5′ splice sites across 1984 clinically relevant OMIM genes.

FIGS. 19 to 55: Data supporting the utility of CSPv7 for prediction of abnormal splice sites in subjects with genetic disorders. CSPv7 was evaluated in a blinded Clinical Validation trial for 400 subject, results for 11 subjects are detailed in FIGS. 19 to 55 with putative splicing variants for whom experimental evidence supporting a prediction of mis-splicing or normal splicing is available. The subset of example cases presented herein demonstrate the interpretative utility and predictive accuracy of CSPv7. Each clinical case presents; 1) the CSPv7 prediction and 2) experimental testing that confirms mis-splicing or normal splicing, as detailed within a Splicing Diagnostic Report (with all confidential information redacted). Data sources the CSPv7 reference database of 14,875 variants affect 9,670 unique 5′ splice sites across 1984 clinically relevant OM IM genes.

FIG. 19: Amplified cDNA products encompassing exons 1-2 and 1-3 of CLN5 in the proband (P) compared to controls (C1, C2) and the parental samples (F, M)

FIG. 20: Sashimi plots showing RNA sequencing (RNAseq) coverage across CC2D2A exons 4-9 (NM_001080522) derived from tibial artery, sigmoid colon, gastroesophageal junction, tibial nerve, lung and cerebellum.

FIG. 21: RT-PCR of CC2D2A mRNA isolated from blood. RT-PCR was performed on mRNA extracted from the whole blood taken from the unaffected parent carriers of the c.438+1G>T variant

FIG. 22: Sanger sequencing of RT-PCR amplicons showed the abnormally sized Band #2 in the maternal and paternal samples was due to exon-7 skipping.

FIG. 23: Schematic of the splicing abnormality induced by the c.438+1G>T variant.

FIG. 24 The c.438+1G>T variant results in exon-7 skipping, an in-frame event. Exon-7 skipping removes 34 amino acids p. (Ser113_Glu146del) from the CC2D2A protein, of which 24 residues are conserved in mammals.

FIG. 25: RT-PCR of PIGN mRNA isolated from blood. FIG. 25 A No abnormal splicing was detected using 3 primer combinations. Intron 4 retention was detected in the patient and three controls (red arrows). FIG. 25 B GAPDH demonstrates similar cDNA loading. Lanes: Patient (P), control 1 (C1) (female, 26 years), control 2 (C2) (female, 27 years), control 3 (C3) (male, 3 weeks).

FIG. 26: Sanger sequencing of RT-PCR amplicons confirmed intron-4 retention in the patient and controls. Levels of intron-4 retention from the c.616+3G>A variant containing allele may be reduced due to the predicted strengthening of the exon-4 5′ splice site. No common SNPs were amplified by our RT-PCRs to investigate allele imbalance.

FIG. 27: Schematic of CACNA1E splicing in blood mRNA.

FIG. 28: Sashimi plots showing RNA sequencing coverage across ASNS exons 9-13 in RNA derived from two brain samples (red, female, 19 weeks; blue, female, 37 weeks); two blood samples (green, male, 49 years; brown, female, 30 years; purple, female, 11 years); and two skin samples (purple, male, 57 years; orange, male, 61 years). ASNS exon-12 is a canonical exon included in all predominant ASNS isoforms expressed in brain, blood and skin.

FIG. 29: RT-PCR of ASNS mRNA isolated from blood. A) Using primers flanking the c.1476+1G>A variant (exon-10 forward and exon-13 reverse) we detected two abnormally sized bands in the patient and parental samples, relative to three controls. Sanger sequencing (FIG. 4) confirmed Band #1 corresponds to use of a cryptic 5′ splice-site, 48 nucleotides upstream of the native 5′ splice-site; and Band #2 corresponds to exon 12 skipping. B) Using a forward primer in exon 12 and a reverse primer in the 3′UTR of ASNS, the proband shows exclusive use of the cryptic 5′ splice-site in exon 12 (Band #3). We find no evidence for normal exon 12 to exon 13 splicing in the affected neonate. Parental samples showed both; 1) normal exon 12 to exon 13 splicing (Band #4) and 2) use of the exon 12 cryptic 5′ splice-site (Band #3), consistent with heterozygosity of the c.1476+1G>A variant. C) Use of a reverse primer in intron 12 shows abnormal inclusion of intronic sequence in the patient, and parental samples, that was not detected in controls. Band #5 corresponds to intron 12 inclusion and Band #6 corresponds to the inclusion of intron 11 and intron 12. D) Amplification of GAPDH demonstrates similar cDNA loading. Lanes: Patient (P), mother (M), father (F), control 1 (C1) (male, 7 months), control 2 (C2) (male, 5 years), control 3 (C3) (Female, 43 years).

FIG. 30: Sanger sequencing of RT-PCR amplicons. A) Chromatogram showing the abnormal sized Band #2 in the patient and parental samples were due to exon-12 skipping. B) Chromatogram showing the abnormal sized Band #1 and #3 in the patient and parental samples were due to the use of the cryptic 5′ splice-site within exon 12. ASNS transcripts with normal splicing from exon 12 to exon 13 were detected in the parental samples, but not detected in the proband.

FIG. 31: Schematic of the splicing abnormalities induced by the c.1476+1G>A variant.

FIG. 32: Sashimi plots showing RNA sequencing (RNAseq) coverage across ARMC4 exons 11-14 in RNA derived from cerebellum, lung and sigmoid colon. ARMC4 exon-12 is included in the predominant isoform and exon-12 skipping is a normal low frequency event. RNAseq data obtained from the Genotype-Tissue Expression (GTEx) Project.

FIG. 33: RT-PCR of ARMC4 mRNA isolated from skin. A) Using two sets of primers flanking the c.1743+5G>C variant we detect three amplicons: Band #1: Normal exon-11-12-13 splicing (paternal and control samples). Band #2: Heteroduplex (controls only). Band #3: Exon-12 skipping (paternal and control samples).

FIG. 34: Sanger sequencing of RT-PCR amplicons. A) In the paternal sample: Band #1 corresponds to normal splicing Band #3 corresponds to exon-12 skipping B) and C) In control samples: Band #1 corresponds to normal splicing Band #2 is a heteroduplex of DNA consisting of normal splicing and exon-12 skipping Band #3 corresponds to exon-12 skipping Band #4 corresponds to intron-12 retention.

FIG. 35: Schematic of ARMC4 splicing and coordinates of the c.1743+5G>C variant. The predominant ARMC4 isoforms splice exon-10-11-12-13-14 sequentially.

FIG. 36: ARMC4 exon-12 amino acid conservation from mammals to fruitfly.

FIG. 37: RT-PCR of AHI1 mRNA isolated from blood. RT-PCR using primers in exons 16 and 19 of AHI1. The c.2492+5G>A variant induces exon 18 skipping (yellow arrow) and use of a cryptic donor (red arrow). Lanes: Patient (P), mother (M), father (F) control 1 (C1), control 2 (C2).

FIG. 38: Schematic of AH11 splicing

FIG. 39: RT-PCR of TAZ mRNA isolated from blood. A) Several abnormally sized bands were detected in the patient sample (P), relative to four control samples (C1-C4). No normally spliced products were detected in the patient sample (P) using a forward primer in exon-1 and a reverse primer in exon-4 of TAZ. B) No product was detected in the patient sample (P) using a forward primer in the 5′UTR and a reverse primer in exon-2 of TAZ, indicating exon-2 spliced into the TAZ at very low levels (exon-2 skipping). C) Amplification of GAPDH demonstrates similar cDNA loading. Lanes: Patient (P), mother (M), father (F) control 1 (C1) (male, 4 years), control 2 (C2) (male, 38 years), control 3 (C3) (female, adult), control 4 (C4) (female, 43 years).

FIG. 40: RT-PCR of TAZ mRNA isolated from myocardium. Several abnormally sized bands were detected in the patient sample (P), relative to two disease control samples (C5, C6). No normally spliced products were detected in the patient sample (P) using forward primers in the 5′UTR and exon-1, and a reverse primer in exon-4 of TAZ. Amplification of GAPDH demonstrates similar cDNA loading. Lanes: Patient (P), control 5 (C5) (32 years), control 6 (C6) (female, 10 years).

FIG. 41: Schematic of the splicing abnormalities induced by the c.238G>C variant.

FIG. 42: RT-PCR of LAMP2 mRNA isolated from blood. A) Using two sets of primers flanking the c.928+3A>T variant we detect a single band corresponding to exon-7 skipping in the proband and affected sibling mRNA (Band #1). In two controls we detect a single band corresponding to normal exon-6-7-8-splicing (Band #2). B) Using a forward primer in exon-4 and a reverse primer in exon-7 we are unable to detect any transcripts containing exon-7 in the proband or affected sibling. C) Using a reverse primer in intron-7, designed to detect use of a potential cryptic 5′ splice site upstream of the native exon-7 5′ splice site, we found no evidence of abnormal splicing. D) Amplification of GAPDH demonstrates cDNA loading. Lanes: Proband (P), Sibling (S) (male, 3 years), Control 1 (01) (male, 7 months), Control 2 (C2) (male, 5 years). Replicate samples were subject to PCR for 25 or 30 cycles in order to confirm the PCR cycling conditions were sub-saturating and able to detect lower levels or quality of a specimen.

FIG. 43: Sanger sequencing of RT-PCR amplicons.

FIG. 44: Schematic of splicing abnormality induced by the c.928+3A>T variant.

FIG. 45: RT-PCR of OPHN1 mRNA isolated from blood. A) Abnormally sized bands were detected in the patient and maternal samples relative to two control samples. B) No product was detected in the patient sample using a forward primer bridging the exon-7/exon-8 junction to specifically probe for normally spliced transcripts. C) Amplification of GAPDH demonstrates similar cDNA loading. Lanes: Patient (P), mother (M), control 1 (C1) (male, 5 years), control 2 (C2) (female, 26 years).

FIG. 46: Sanger sequencing of RT-PCR amplicons confirmed the abnormal sized bands in the patient and mother samples were due to exon-8 skipping. Normally spliced OPHN1 transcripts were also detected in the maternal sample.

FIG. 47: Schematic of exon-8 skipping induced by the c.702+4A>G variant.

FIG. 48: RT-PCR of HSD17B4 mRNA isolated from patient lymphoblasts. A)-C) Primers flanking the c.1333+1G>C variant amplified an abnormal lower band in the patient sample (red arrows). Sanger sequencing confirmed these amplicons correspond with exon-15 skipping. Yellow arrows: RT-PCR amplicon with normal exon-14-exon-15-exon-16 splicing was also detected in patient RNA, confirmed by Sanger sequencing, and presumably derived from the HSD17B4 allele bearing the c.46G>A variant. D) Using a forward primer (Ex14/16-F) designed to anneal with the exon-14-exon-16 junction we were able to specifically amplify HSD17B4 transcripts that skipped exon-15. Levels of exon-15 skipping are notably higher in the patient mRNA relative to two controls. E) GAPDH demonstrates similar cDNA loading. Lanes: Patient (P), control 1 (C1) (PBMC mRNA, female, 43 years), control 2 (C2) (PBMC mRNA, female, 37 years), control 3 (C3) (PHF mRNA, female, 7 years), control 4 (C4) (PHF mRNA, female, 53 years).

FIG. 49: Sanger sequencing of RT-PCR amplicons confirm exon-15 skipping in HSD17B4 transcripts of the patient mRNA.

FIG. 50: RT-PCR of ACE mRNA isolated from whole blood. A) Using primers flanking the c.1709+5G>C variant we detected 2 bands: Band #1 and Band #3: normally spliced ACE transcripts Band #2 and Band #4: exon 11 skipping (only detected in the maternal and paternal samples). B) We used a forward primer designed to anneal with the exon 10-exon 12 junction to specifically amplify ACE transcripts with exon 11 skipping. Exon 11 skipping was only observed in the maternal and paternal mRNA samples (Band #5), and was not detected in two controls. C) Amplification of GAPDH demonstrates cDNA loading. Lanes: Mother (M), Father (F), Control 1 (C1) (Female, 36 years), Control 2 (C2) (Male, 39 years). We also detect normal splicing of ACE transcripts in the maternal and paternal samples.

FIG. 51. Sanger sequencing of RT-PCR amplicons. Sequencing showed the abnormally sized Band #2 (FIG. 2A) in the maternal and paternal samples was due to exon 11 skipping.

FIG. 52: RT-PCR of ACE mRNA isolated from fibroblasts (i) and renal epithelia (ii). A) Using primers flanking the c.1709+5G>C variant we detected three bands: Band #1: normally spliced ACE transcripts (paternal sample and controls) Band #2 Heteroduplex amplicon (paternal sample only) DSMO: contains a mix of normally spliced transcripts and exon 11 skipping CHX: contains normally spliced transcripts, exon 11 skipping and use of a cryptic 5′-splice site Band #3: exon 11 skipping (only detected in the paternal sample). B) We used a forward primer designed to anneal with the exon 10-exon 12 junction to specifically amplify ACE transcripts with exon 11 skipping. Exon 11 skipping was only observed in the paternal mRNA samples (Band #4), and was not detected in two controls. C) Amplification of GAPDH demonstrates cDNA loading. Lanes: i) Father (F), Control 1 (C1) (Male, 52 years), Control 2 (C2) (Male, 49 years). ii) Father (F), Control 1 (C1) (Male, 30 years).

FIG. 53: Sanger sequencing of RT-PCR amplicons from fibroblasts (A) and renal epithelia (B).

FIG. 54: Schematic of splicing abnormalities induced by the c.1709+5G>C variant.

FIG. 55: ACE exon 11 amino acid conservation between mammals, birds, amphibians and fish.

FIG. 56: Embodiment supporting search of cryptic splice sites. Illustrated example represents search for consecutive cryptic site sequences having the essential splice site “GT” or “GC” bases and 12 nucleotides length within two adjacent regions of the genome (typically exon and intron). Potential use of cryptic splice site is evaluated by comparing cryptic splice site sequence's median NIFᵥₐᵣ₋ₓ or median percentile NIFᵥₐᵣ₋ₓ with authentic donor's median NIFᵥₐᵣ or median percentile NIFᵥₐᵣ.

FIG. 57: Embodiment supporting search for variants affecting same donor 5′ splice-site. Illustrated example represents search for CSP reference database variants that reside within a certain distance from the sample variant.

### BRIEF DESCRIPTION OF THE TABLES

Table 1: (above) Four exemplary embodiments relating to embodiments comprising at least six sample donor splice site sequences from a sample donor splice site are depicted in Table 1 wherein the nucleotides of a sample donor splice site are indicated as nucleotide positions E⁻⁵ to D⁺⁹ and an “x” indicates that that nucleotide is included in a sample donor splice site sequence.

Table 2: Blinded trial of Clinical Splice Predictor (V3) for BRCA1 or BRCA2 variants identified in individuals with breast cancer, with experimental confirmation of splicing outcomes. Clinical Splice Predictor reports were analysed blinded for thirty putative splice variants identified in cancer oncogenes BRCA1 and BRCA2. Genomic variants were classified according to defined criteria (see Table 4). Unblinding to published experimental outcomes reveals 100% predictive accuracy for BRCA1 and BRCA2 True Positive (abnormal splice sites) variant splice sites and True Negative (benign variant splice sites) variant splice sites.

TABLE 2
Blinded trial of Clinical Splice Predictor (V3): BRCA1 and BRCA2 variants with experimental confirmation of splicing outcomes.

| | | | | CSP | | | Experimentally-determined | |
|---|---|---|---|---|---|---|---|---|
| Case | Gene | Variant | Ref | Report | Class^{fcn}. | Definition | splicing outcomes | Agree |
| 1 | BRCA1 | NM_007294.3:c.4986 + 1G > T | [1] | 4266 | Class 5 | High confidence extreme | Use of cryptic splice site. | Yes |
| | | | | | | risk of abnormal splicing | Insertion 65 nt intron | |
| 2 | BRCA1 | NM_007294.3:c.4986 + 5G > T | * | 4267 | Class 5 | High confidence extreme | Use of cryptic splice site. | Yes |
| | | | | | | risk of abnormal splicing | Insertion 65 nt intron | |
| 3 | BRCA1 | NM_007294.3:c.4986 + 5G > A | [1] | 4268 | Class 5 | High confidence extreme | Use of cryptic splice site. | Yes |
| | | | [2] | | | risk of abnormal splicing | Insertion 65 nt intron | |
| 4 | BRCA1 | NM_007294.3:c.5152 + 1G > C | [2] | 4269 | Class 5 | High confidence extreme | Exon 18 skipping | Yes |
| | | | | | | risk of abnormal splicing | | |
| 5 | BRCA1 | NM_007294.3:c.441 + 2T > G | [1] | 4270 | Class 5 | High confidence extreme | Use of cryptic splice site. | Yes |
| | | | | | | risk of abnormal splicing | Skipping 62 bp 3′ end of exon | |
| | | | | | | | 7 | |
| 6 | BRCA1 | NM_007294.3:c.547 + 2T > A | [1] | 4271 | Class 5 | High confidence extreme | Exon 8 skipping | Yes |
| | | | | | | risk of abnormal splicing | | |
| 7 | BRCA1 | NM_007294.3:c.5332 + 1G > A | [1] | 4272 | Class 5 | High confidence extreme | Exon 21 skipping | Yes |
| | | | | | | risk of abnormal splicing | | |
| 8 | BRCA1 | NM_007294.3:c.4484G > T | [1] | 4275 | Class 3B | VUS with tangible risk of | Exon 14 skipping | Yes |
| | | | [3] | | | abnormal splicing | | |
| 9 | BRCA1 | NM_007294.3:c.4185G > A | [2] | 4277 | Class 5 | High confidence extreme | Exon 12 skipping. | Yes |
| | | | | | | risk of abnormal splicing | Synonymous Q1395Q | |
| 10 | BRCA1 | NM_007294.3:c.5193 + 2T > G | [2] | 4278 | Class 5 | High confidence extreme | Exon 19 skipping | Yes |
| | | | | | | risk of abnormal splicing | | |
| 11 | BRCA1 | NM_007294.3:c.5406 + 3A > T | [2] | 4279 | Class 4A | High risk of abnormal | Exon 22 skipping | Yes |
| | | | | | | splicing | | |
| 12 | BRCA1 | NM_007294.3:c.5406 + 4A > G | [2] | 4280 | Class 4B | Very high risk of abnormal | Exon 22 skipping | Yes |
| | | | | | | splicing | | |
| 13 | BRCA1 | NM_007294.3:c.4986 + 3G > C | [2] | 4281 | Class 4B | Very high risk of abnormal | Insertion 65 nt intron | Yes |
| | | | | | | splicing | | |
| 14 | BRCA1 | NM_007294.3:c.4986 + 4A > G | [2] | 4282 | Class 5 | High confidence extreme | Insertion 65 nt intron | Yes |
| | | | | | | risk of abnormal splicing | | |
| 15 | BRCA1 | NM_007294.3:c.4675G > A | [2] | 4283 | Class 4B | Very high risk of abnormal | Use of cryptic splice site. | Yes |
| | | | | | | splicing | Removes 11 nt 3′ of exon | |
| | | | | | | | 15. Missense E1559K | |
| 16 | BRCA1 | NM_007294.3:c.591C > T | [3] | 4285 | Class 3A | Evidence consistent with | Normal Splicing | Yes |
| | | | | | | normal splicing | | |
| 17 | BRCA2 | NM_000059.3:c.681 + 5G > C | # | 4286 | Class 5 | High confidence extreme | Abnormal Splicing | Yes |
| | | | | | | risk of abnormal splicing | | |
| 18 | BRCA2 | NM_000059.3:c.475 + 1G > A | [1] | 4287 | Class 5 | High confidence extreme | Exon 5 skipping | Yes |
| | | | | | | risk of abnormal splicing | | |
| 19 | BRCA2 | NM_000059.3:c.631G > A | [1] | 4288 | Class 4A | High risk of abnormal | Exon 7 skipping | Yes |
| | | | | | | splicing | | |
| 20 | BRCA2 | NM_000059.3:c.8754 + 3G > C | [1] | 4289 | Class 4A | High risk of abnormal | Use of cryptic splice site. | Yes |
| | | | | | | splicing | Retention 46 bp from 5′ | |
| | | | | | | | end of intron 21 | |
| 21 | BRCA2 | NM_000059.3:c.9116C > T | [1] | 4290 | Class 2 | Normal splicing likely | Normal Splicing | Yes |
| 22 | BRCA2 | NM_000059.3:c.9117G > A | [1] | 4291 | Class 4A | High risk of abnormal | Exon 23 skipping | Yes |
| | | | [4] | | | splicing | | |
| 23 | BRCA2 | NM_000059.3:c.8486A > T | [4] | 4292 | Class 3B | VUS with tangible risk | 80% Exon 19 skipping. | Yes |
| | | | | | | of abnormal splicing | 20% Normal splicing. | |
| 24 | BRCA2 | NM_000059.3:c.8754G > A | [4] | 4293 | Class 4A | High risk of abnormal | Use of cryptic splice site. | Yes |
| | | | | | | splicing | Ivs21- ins46 (100%) | |
| 25 | BRCA2 | NM_000059.3:c.8754 + 5G > T | [4] | 4294 | Class 4A | High risk of abnormal | Use of cryptic splice site. | Yes |
| | | | | | | splicing | Ivs21- ins46 (100%) | |
| 26 | BRCA2 | NM_000059.3:c.8754 + 5G > A | [4] | 4295 | Class 4A | High risk of abnormal | Use of cryptic splice site. | Yes |
| | | | | | | splicing | Ivs21- ins46 (100%) | |
| 27 | BRCA2 | NM_000059.3:c.8754 + 4A > G | [4] | 4296 | Class 4A | High risk of abnormal | Use of cryptic splice site. | Yes |
| | | | | | | splicing | Ivs21- ins46 (100%) | |
| 28 | BRCA2 | NM_000059.3:c.9501 + 3A > T | [4] | 4297 | Class 3B | VUS with tangible risk | 87% Normal Splicing. | Yes |
| | | | | | Class 4A | of abnormal splicing | 13% Exon 25 skipping. | |
| | | | | | | High risk of abnormal | | |
| | | | | | | splicing | | |
| 29 | BRCA2 | NM_000059.3:c.9256 + 1G > A | [4] | 4299 | Class 5 | High confidence extreme | 74% Exon 24 skipping. | Yes |
| | | | | | | risk of abnormal splicing | 26% Cryrptic splice site | |
| | | | | | | | Exon 24 del43. | |
| 30 | BRCA2 | NM_000059.3:c.8953 + 1G > T | [4] | 4298 | Class 5 | High confidence extreme | 44% Exon 22 skipping. | Yes |
| | | | | | | risk of abnormal splicing | 39% intron 22 retention. | |

[1] Colombo et al., doi: 10.1371/journal.pone.0057173; PMID: 23451180
[2] Wappenschimidt et al., doi: 10.1371/journal.pone.0050800; PMID: 23239986
[3] Santos et al., http://dx.doi.org/10.1016/j.jmoldx.2014.01.005; PMID: 24607278
[4] Acedo et al., DOI: 10.1002/humu.22725; PMID: 25382762
* PMID: 15604628; 17508274; 18163131; 18693280; 20301425; 23788249; 24366376; 24366402; 24432435; 27854360
# PMID: 23788249; 25394175; 26780556; 27854360

Overall Predictive accuracy:

### 30/30 True Positive and True Negative Predicted Accurately

### REFERENCES

1. Colombo, M., et al., Comparative in vitro and in silico analyses of variants in splicing regions of BRCA1 and BRCA2 genes and characterization of novel pathogenic mutations. PLoS One, 2013, 8(2): p. e57173.
2. Wappenschmidt, B., et al., Analysis of 30 putative BRCA1 splicing mutations in hereditary breast and ovarian cancer families identifies exonic splice site mutations that escape in silico prediction. PLoS One, 2012, 7(12): p. e50800.
3. Santos, C., et al., Pathogenicity evaluation of BRCA1 and BRCA2 unclassified variants identified in Portuguese breast/ovarian cancer families. J Mol Diagn, 2014, 16(3): p. 324-34.
4. Acedo, A., et al., Functional classification of BRCA2 DNA variants by splicing assays in a large minigene with 9 exons. Hum Mutat, 2015, 36(2): p. 210-21.

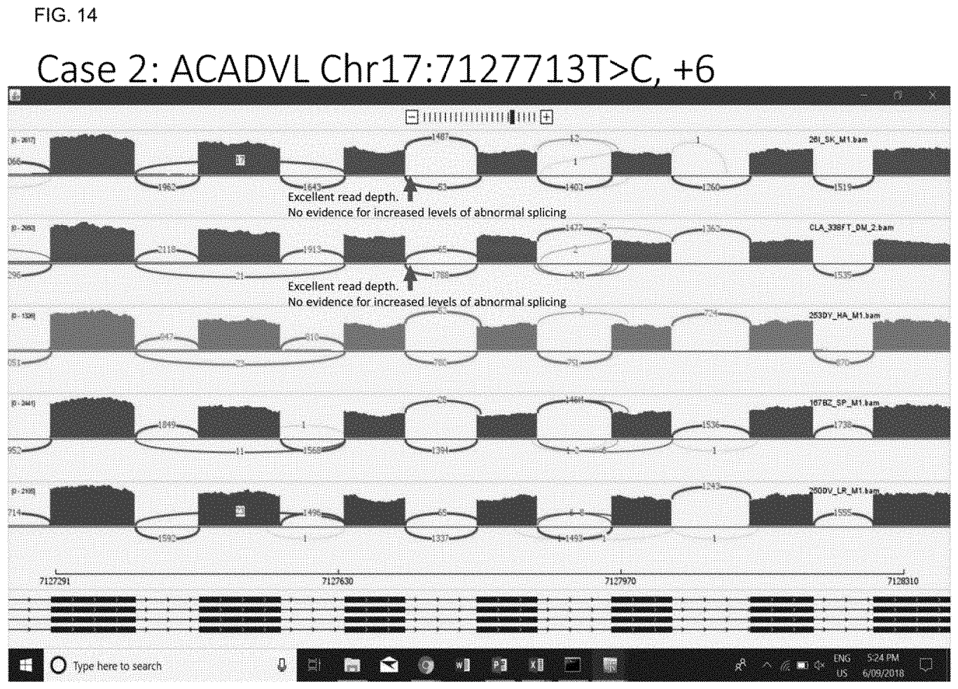

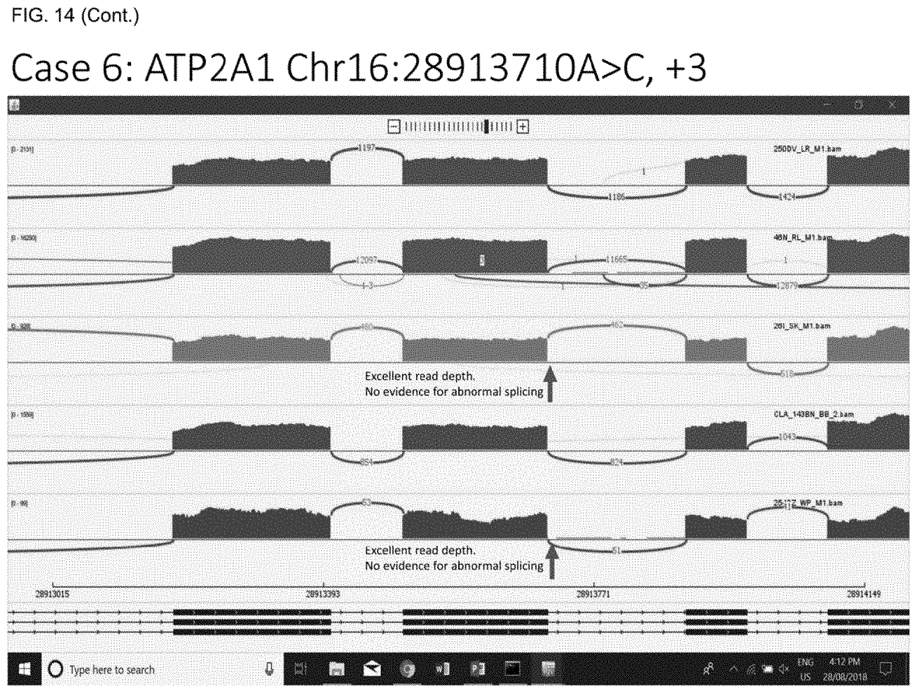

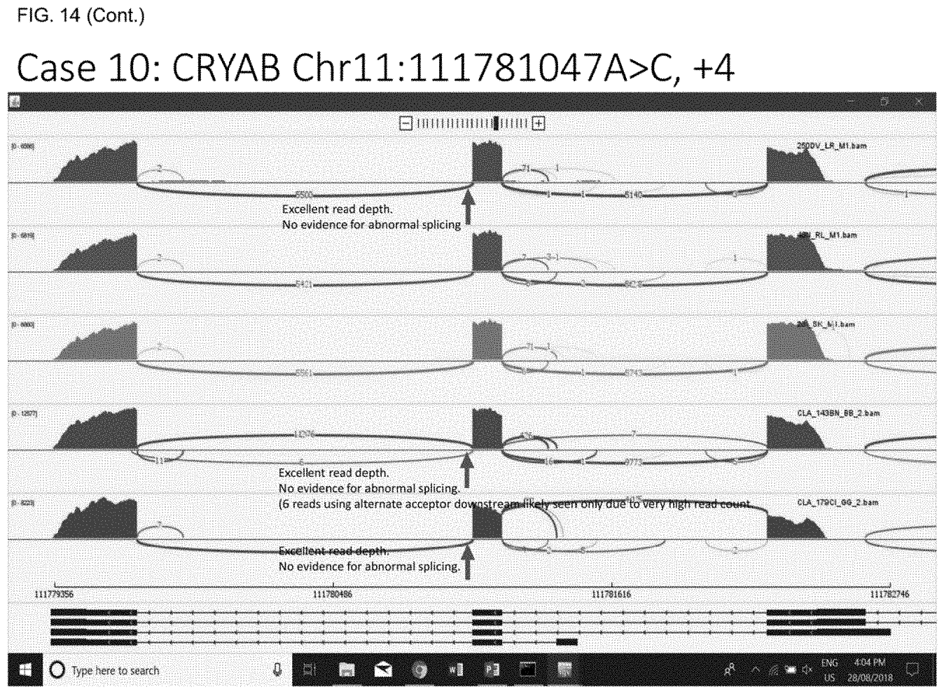

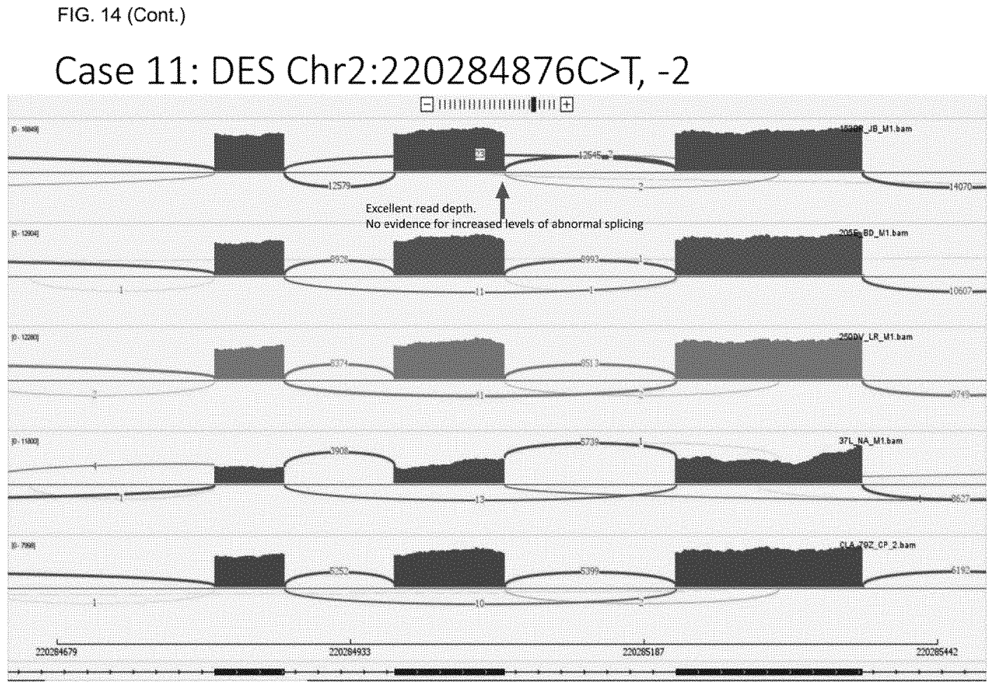

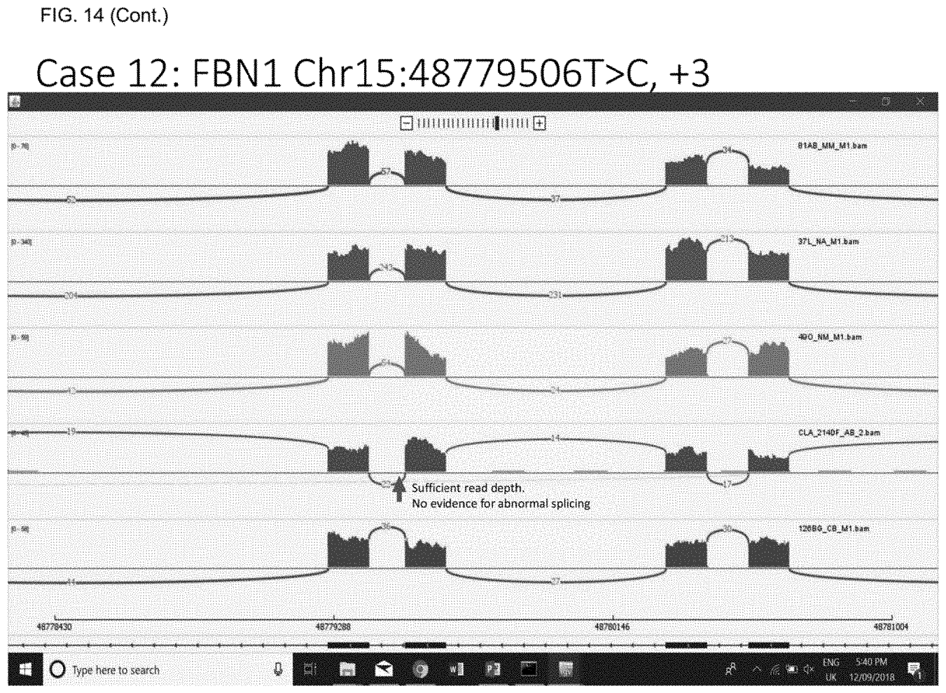

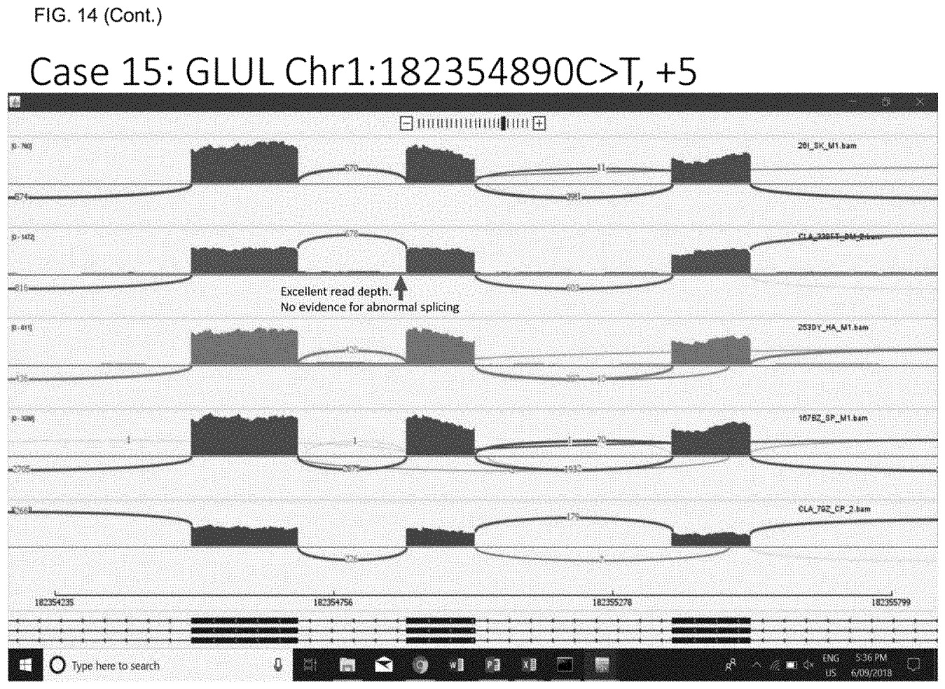

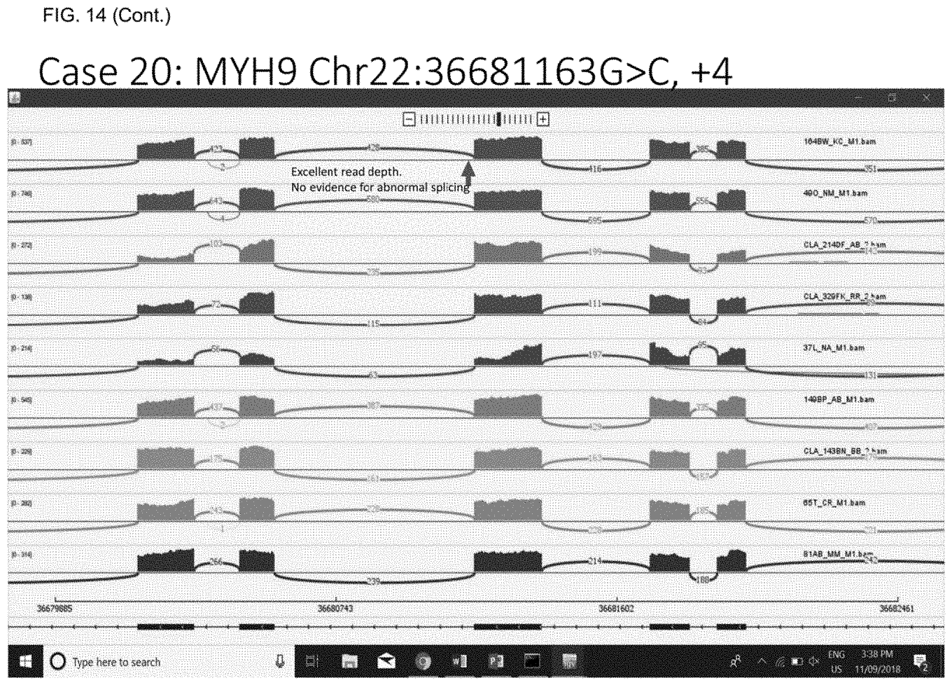

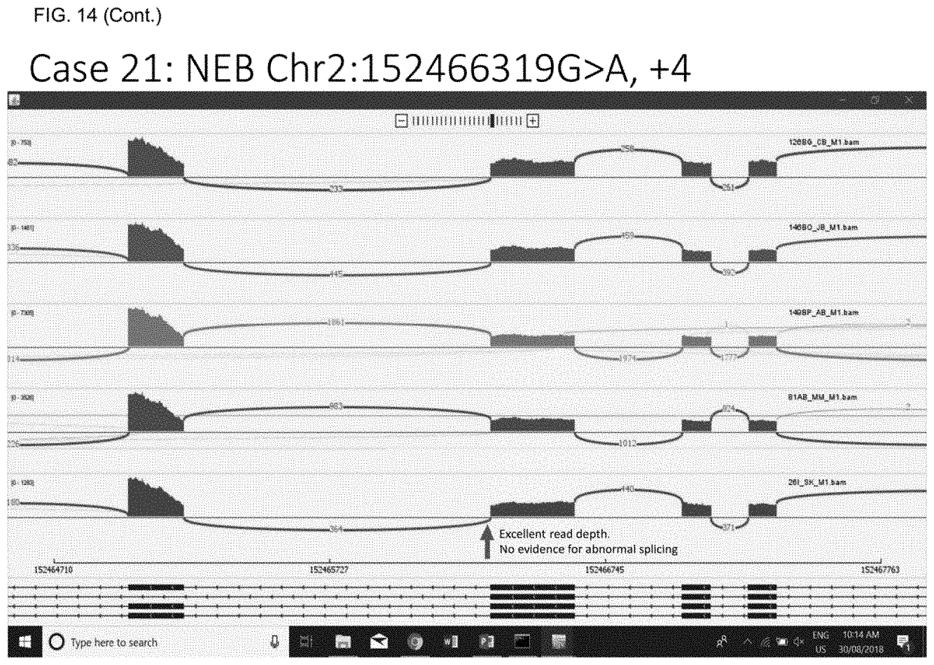

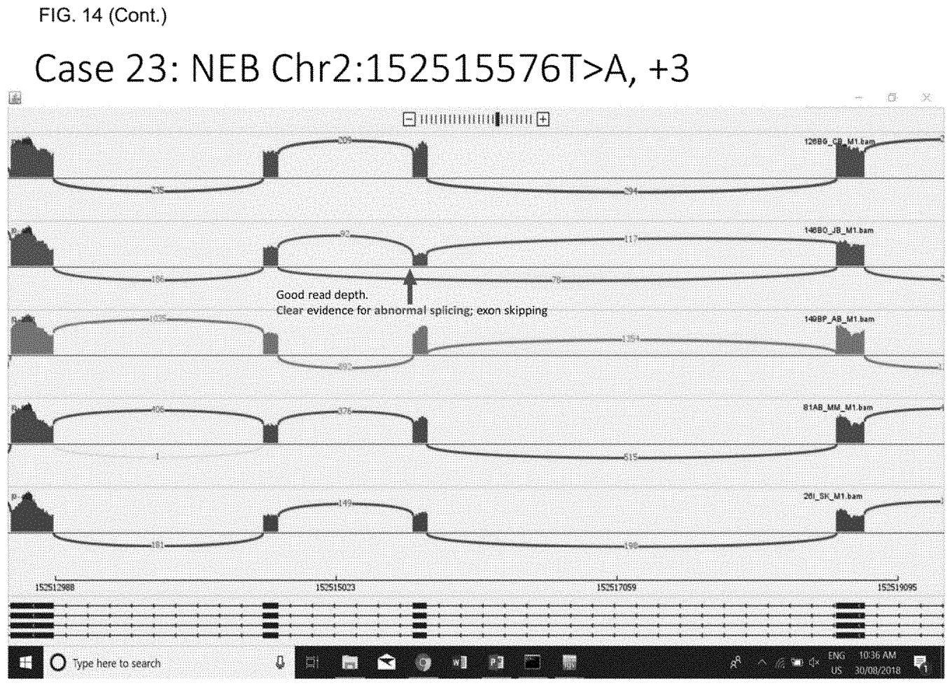

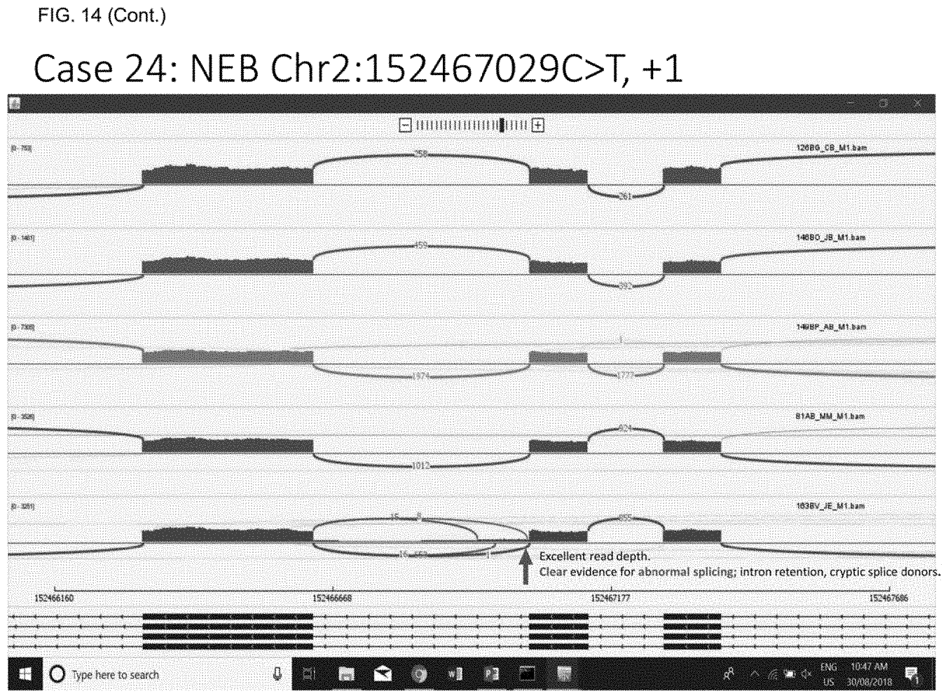

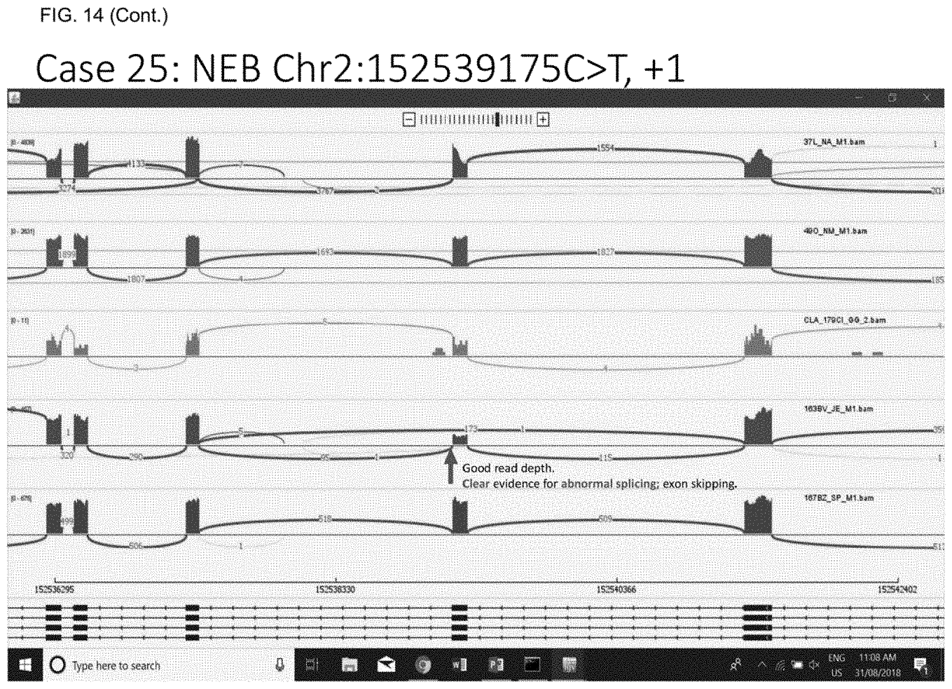

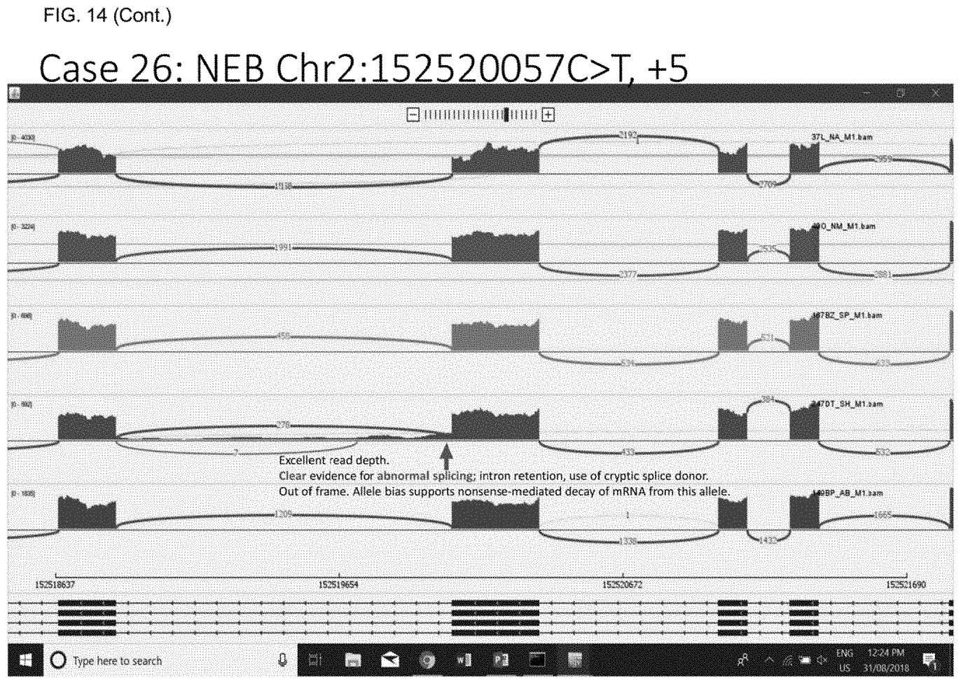

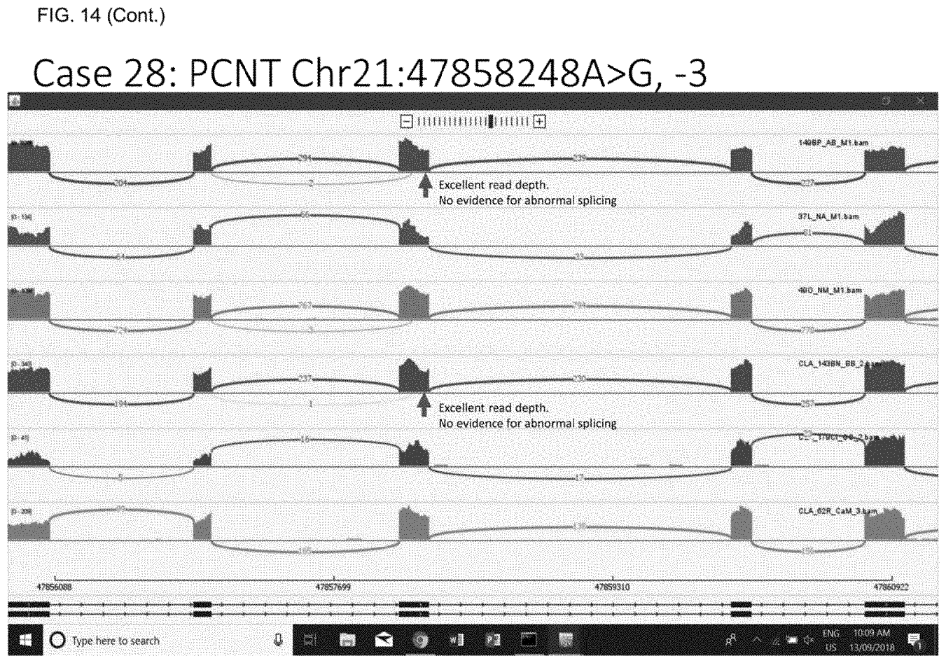

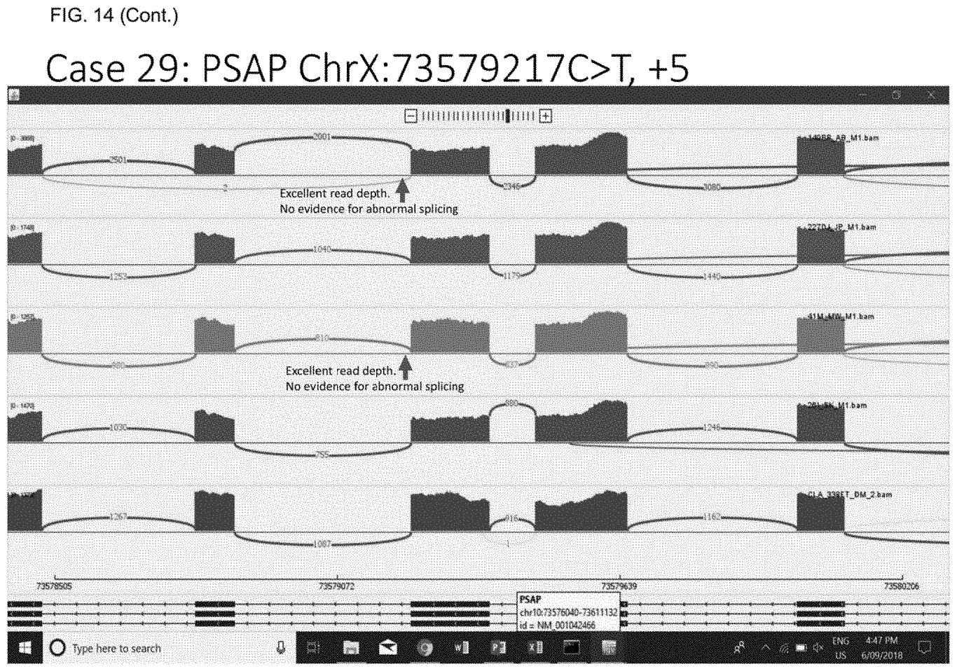

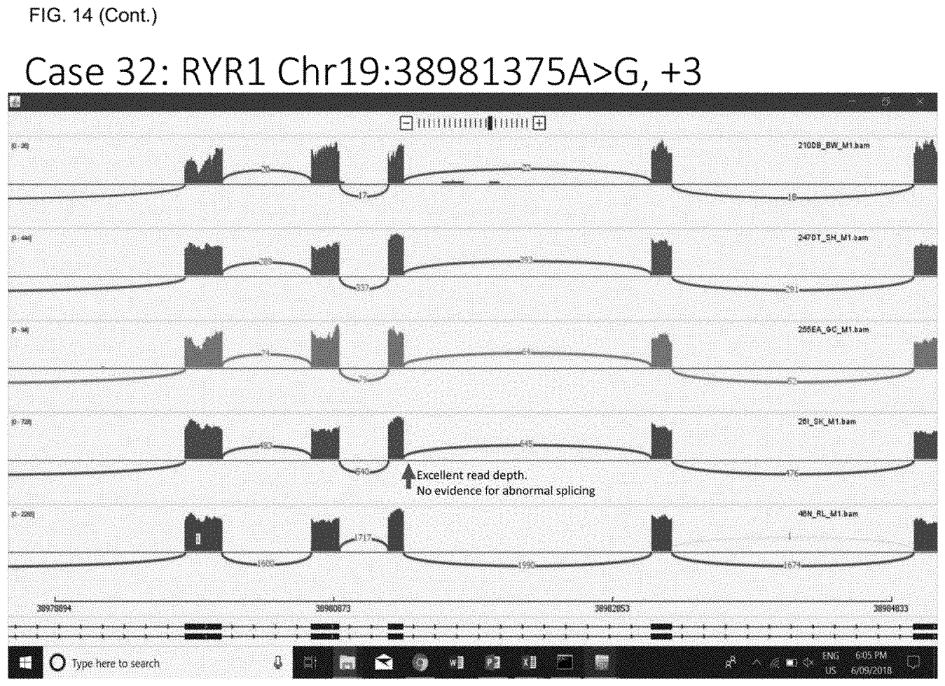

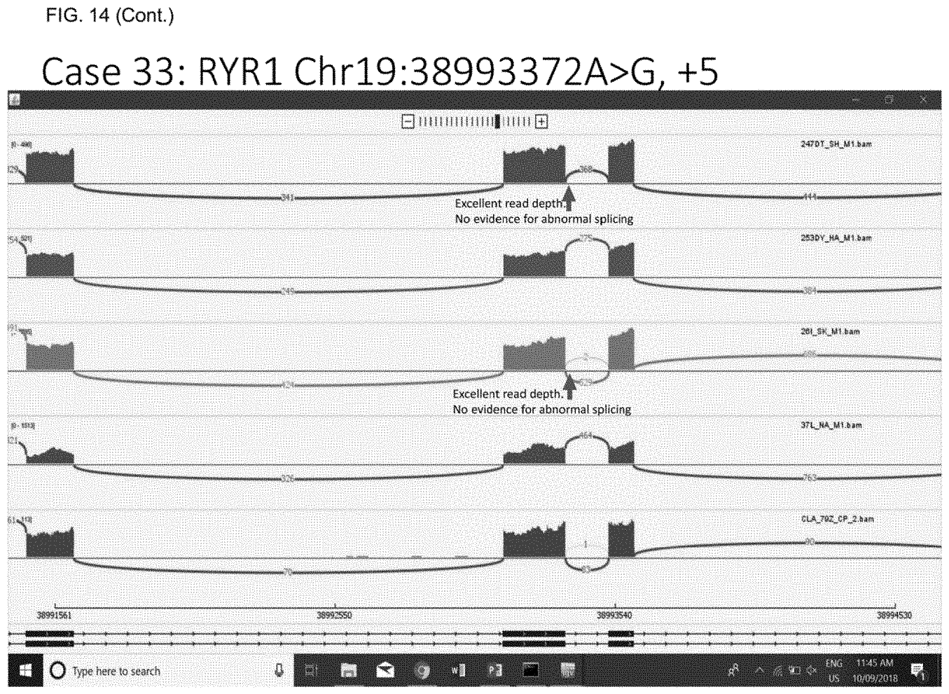

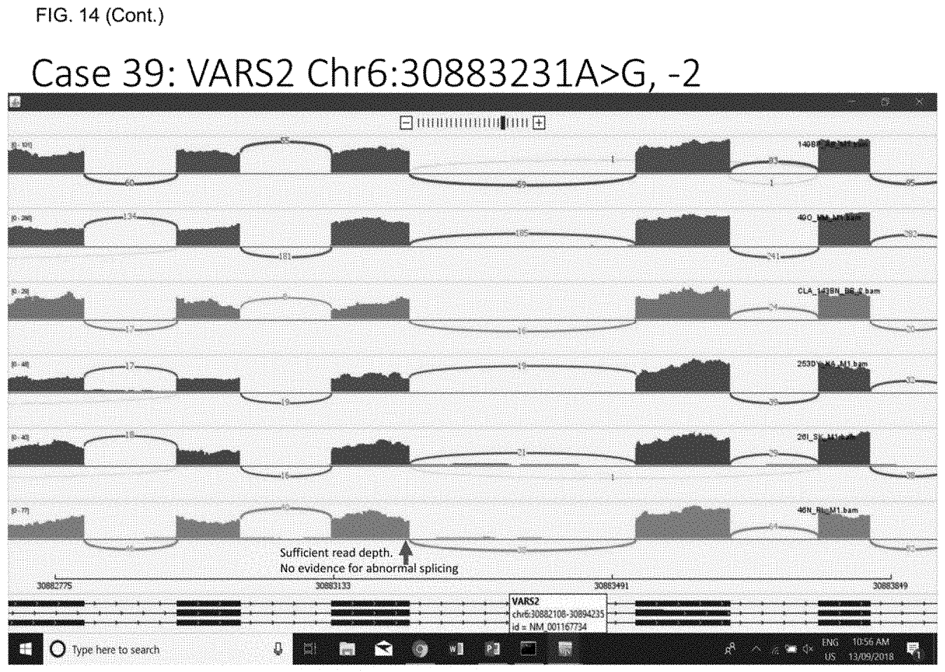

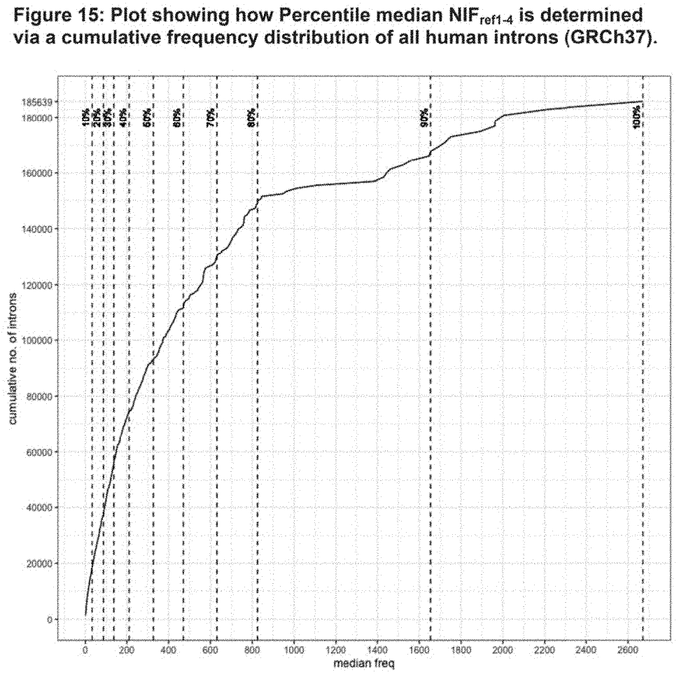

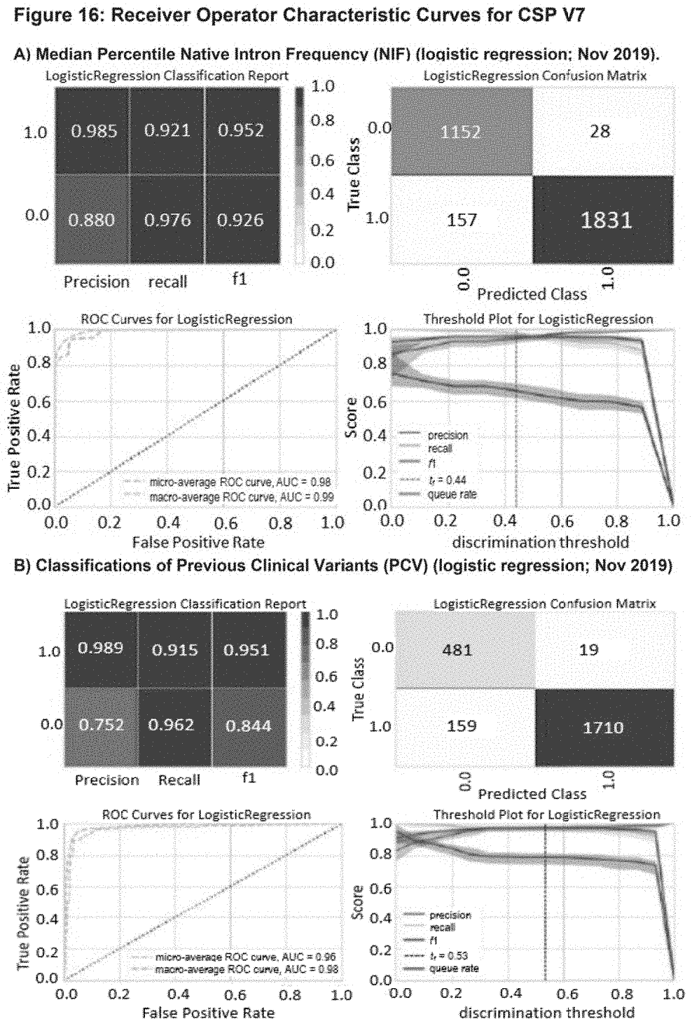

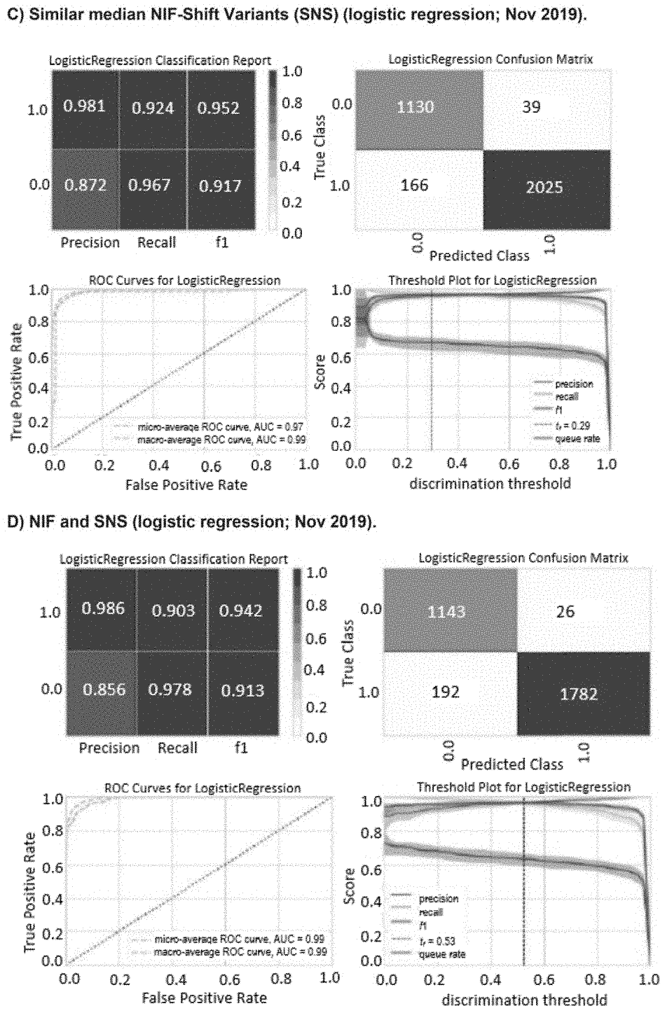

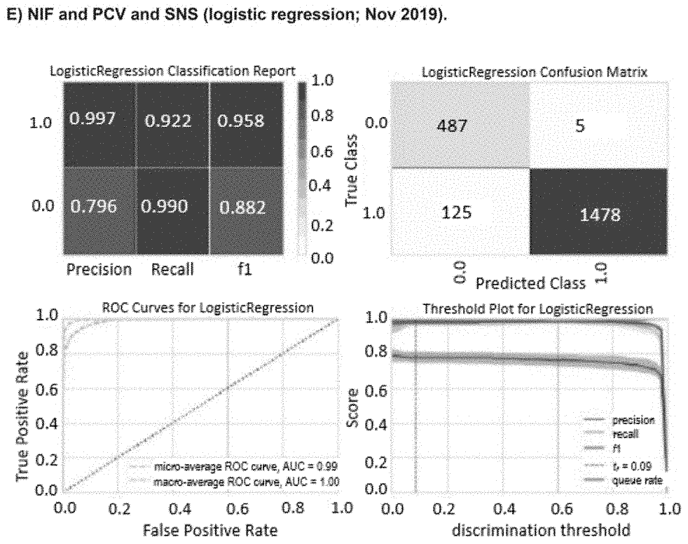

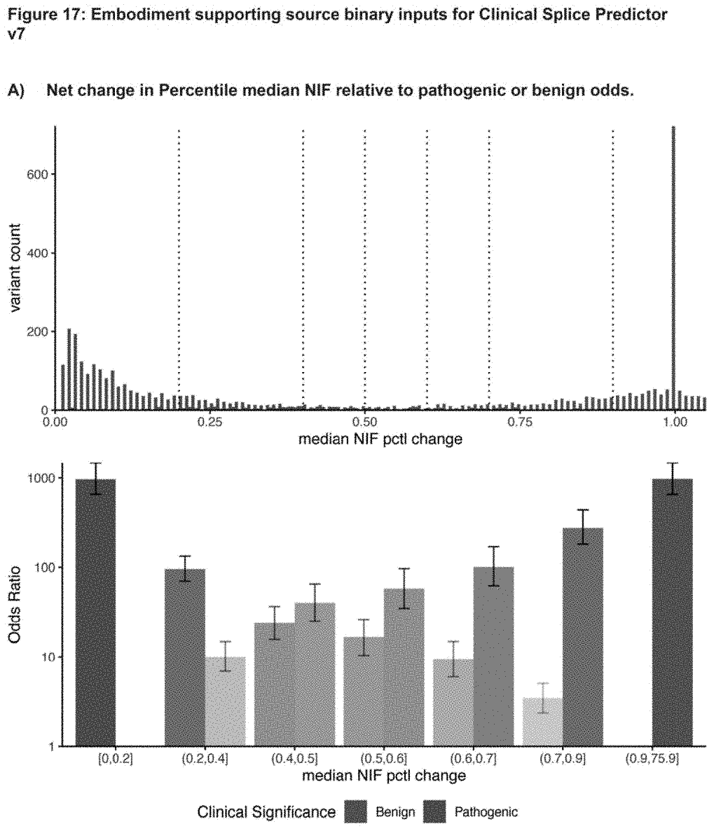

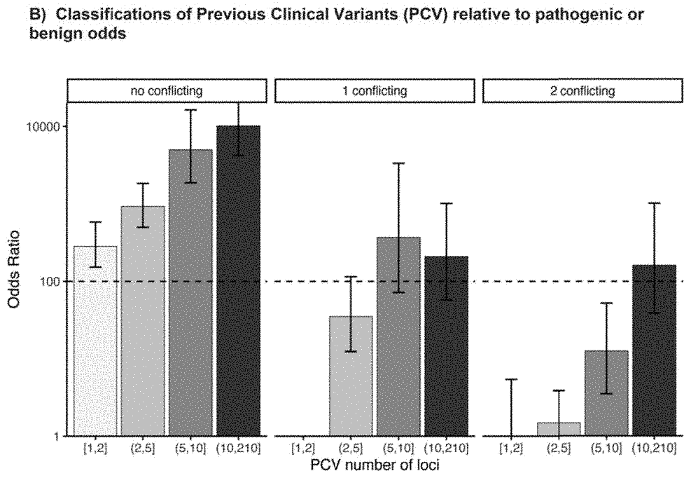

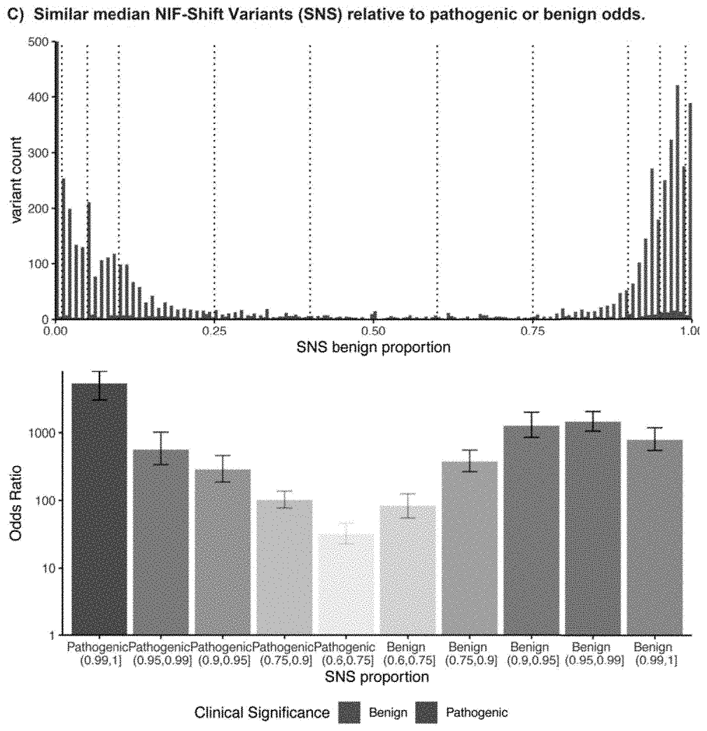

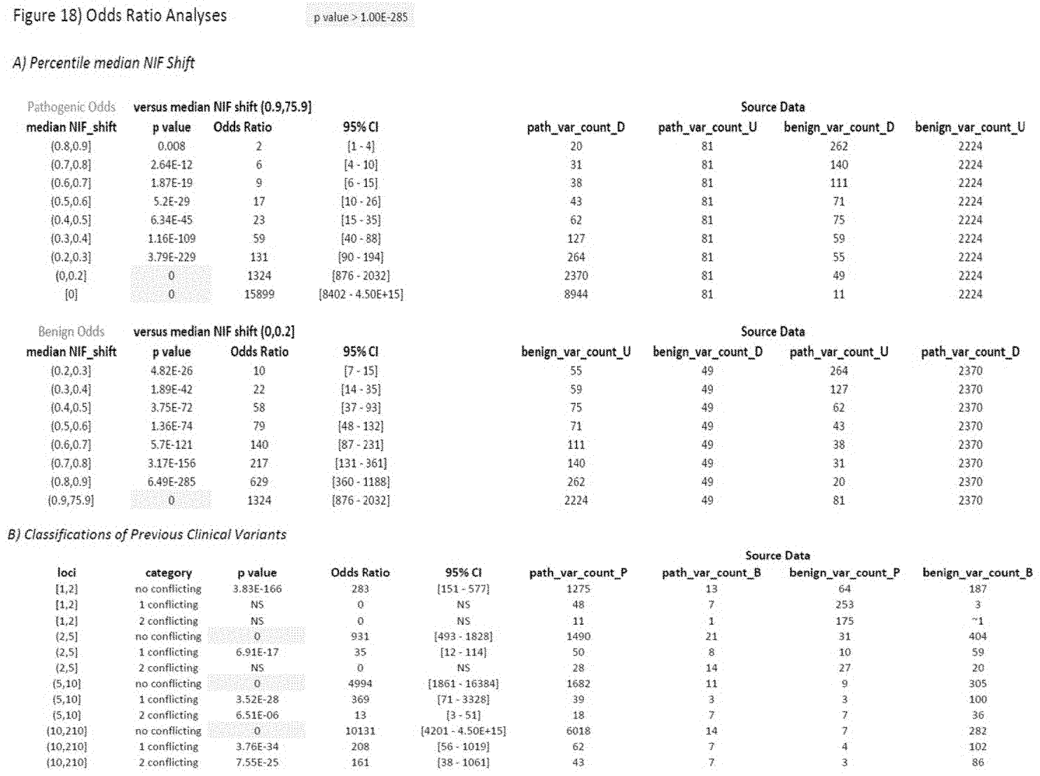

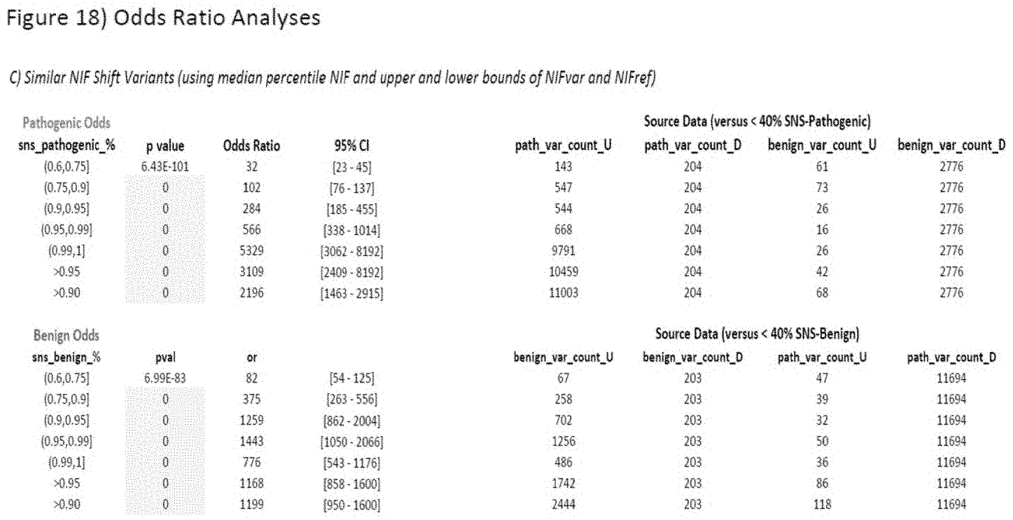

Table 3: Blinded trial of Clinical Splice Predictor (V3) for putative splice variants across all fields of genomic medicine, with RNA-sequencing providing confirmation of splicing outcomes. Clinical Splice Predictor reports were analysed blinded for thirty-nine putative splice variants identified in a range of OM IM genes associated with different Mendelian disorders. Genomic variants were classified according to defined criteria (see Table 4). Unblinding to RNA-sequencing experimental outcomes reveals 100% predictive accuracy for True Positive (abnormal splice sites) variant splice sites and True Negative (benign variant splice sites) variant splice sites. See also FIG. 14.

TABLE 3
Blinded trial of Clinical Splice Predictor (V3): All genetic conditions with experimental confirmation of splicing outcomes by RNA-Sequencing.

| | | | | Donor | Report | | | | | |
|---|---|---|---|---|---|---|---|---|---|---|
| Case | Phenotype | Gene | Variant | Posⁿ. | No. | Class^{fcn}. | Definition | RNA-Seq | Accurate | NOTES |
| 1 | Short chain acyl-CoA | ACADS | NM_000017.3 | −1 | BV-00001 | Class 1 | High confidence of normal | Normal | Yes | |
| | dehydrogenase deficiency | | exon 3 | | | | splicing | Splicing | | |
| 2 | Very long chain acyl-CoA | ACADVL | NM_000018.3 | +6 | BV-00002 | Class 1 | High confidence of normal | Normal | Yes | |
| | dehydrogenase deficiency | | intron 16 | | | | splicing | Splicing | | |
| 3 | Retinitis pigmentosa | ARHGEF18 | NM_015318.3 | −3 | BV-00005 | Class 2 | Normal splicing likely | Normal | Yes | |
| | | | exon 4 | | | | | Splicing | | |
| 4 | Retinitis pigmentosa | ARHGEF18 | NM_015318.3 | +6 | BV-00006 | Class 1 | High confidence of normal | Normal | Yes | |
| | | | intron 17 | | | | splicing | Splicing | | |
| 5 | Retinitis pigmentosa | ARHGEF18 | NM_015318.3 | +4 | BV-00008 | Class 2 | Normal splicing likely | Normal | Yes | |
| | | | intron 3 | | | | | Splicing | | |
| 6 | BRODY | ATP2A1 | NM_004320.4 | +3 | BV-00009 | Class 4A | High risk of abnormal | 95% Normal | No/Yes | Excellent read depth. |
| | MYOPATHY | | intron 17 | | | | splicing | Splicing | | Analyzed het/z and hom/ |
| | | | | | | | | 5% abnormal | | z. Very low levels of |
| | | | | | | | | splicing | | abnormal splicing (intron |
| | | | | | | | | | | retention, all abnormal |
| | | | | | | | | | | splicing events have |
| | | | | | | | | | | variant +3). Vast |
| | | | | | | | | | | majority normal splicing. |
| 7 | Lethal neonatal spasticity- | BRAT1 | NM_152743.3 | −2 | BV-00011 | Class 2 | Normal splicing likely | Normal | Yes | |
| | epileptic encephalopathy | | exon 10 | | | | | Splicing | | |
| | syndrome | | | | | | | | | |
| 8 | Lethal neonatal spasticity- | BRAT1 | NM_152743.3 | −1 | BV-00012 | Class 3B | VUS; tangible risk of | 65% normal | Yes | Low read depth. 4/9 reads |
| | epileptic encephalopathy | | exon 1 | | | | abnormal splicing | splicing | | use alt. donor +5 into |
| | syndrome | | | | | | | 35% abnormal | | the intron (GC donor). |
| | | | | | | | | splicing | | This donor is used |
| | | | | | | | | | | in another isoform. |
| | | | | | | | | | | Non-coding 5′UTR. |
| 9 | Childhood absence | CACNA1H | NM_021098.2 | −3 | BV-00013 | Class 2 | Normal splicing likely | Normal | Yes | Low read depth but have |
| | epilepsy | | exon 21 | | | | | Splicing | | RNA-Seq for six carriers. |
| | | | | | | | | | | All normal splicing. |
| 10 | Fatal infantile hypertonic | CRYAB | NM_001885.2 | +4 | BV-00015 | Class 1 | High confidence of normal | Normal | Yes | |
| | myofibrillar myopathy, | | intron 3 | | | | splicing | Splicing | | |
| | Early-onset cataract | | | | | | | | | |
| 11 | AD LGMD 1E, AR LGMD | DES | NM_001927.3 | −2 | BV-00016 | Class 1 | High confidence of normal | Normal | Yes | |
| | type 2R, Dilated | | exon 2 | | | | splicing | Splicing | | |
| | cardiomyopathy. | | | | | | | | | |
| 12 | MARFAN SYNDROME | FBN1 | NM_000138.4 | +3 | BV-00019 | Class 2 | Normal splicing likely | Normal | Yes | |
| | type 1 | | intron 28 | | | | | Splicing | | |
| 13 | Amyotrophic lateral | FIG4 | NM_014845.5 | +3 | BV-00020 | Class 1 | High confidence of normal | Normal | Yes | |
| | sclerosis, Charcot-Marie- | | intron 17 | | | | splicing | Splicing | | |
| | Tooth Type 4). | | | | | | | | | |
| 14 | Autosomal dominant | GARS | NM_002047.3 | +5 | BV-00023 | Class 1 | High confidence of normal | Normal | Yes | |
| | Charcot-Marie-Tooth | | intron 1 | | | | splicing | Splicing | | |
| | disease type 2D | | | | | | | | | |
| 15 | Congenital brain | GLUL | NM_002065.6 | +5 | BV-00024 | Class 2 | Normal splicing likely | Normal | Yes | |
| | dysgenesis due to | | intron 6 | | | | | Splicing | | |
| | glutamine synthetase | | | | | | | | | |
| | deficiency | | | | | | | | | |
| 16 | HEME OXYGENASE 1 | HMOX1 | NM_002133.2 | +4 | BV-00025 | Class 1 | High confidence of normal | Normal | Yes | |
| | DEFICIENCY | | intron 2 | | | | splicing | Splicing | | |
| 17 | Cardiomyopathy dilated | LMNA | NM_170707.3 | −1 | BV-00026 | Class 2 | Normal splicing likely | Normal | Yes | |
| | 1A, EMD muscular | | exon 10 | | | | | Splicing | | |
| | dystrophy, Severe | | | | | | | | | |
| | lipodystrophic | | | | | | | | | |
| | laminopathy, Charcot- | | | | | | | | | |
| | Marie-Tooth type 2B1 | | | | | | | | | |
| 18 | AD Charcot-Marie-Tooth | MARS | NM_004990.3 | +1 | BV-00028 | Class 5 | High confidence extreme | Abnormal | Yes | Patient with myopathy. |
| | disease type 2U, AR spastic | | intron 8 | | | | risk of abnormal splicing | splicing | | Check if neuropthy |
| | paraplegia type 70, | | | | | | | | | feature of phenotype. |
| | | | | | | | | | | This variant could be |
| | | | | | | | | | | disease-causing or |
| | | | | | | | | | | disease-modifier. |
| 19 | CARDIOMYOPATHY, | MYH7 | NM_000257.3 | −1 | BV-00029 | Class 1 | High confidence of normal | Normal | Yes | |
| | Classic multiminicore | | exon 8 | | | | splicing | Splicing | | |
| | myopathy. | | | | | | | | | |
| 20 | AD nonsyndromic | MYH9 | NM_002473.5 | +4 | BV-00033 | Class 2 | Normal splicing likely | Normal | Yes | |
| | sensorineural deafness | | intron 38 | | | | | Splicing | | |
| | type DFNA | | | | | | | | | |
| 21 | NEMALINE MYOPATHY 2 | NEB | NM_001271208.1 | +4 | BV-00034 | Class 1 | High confidence of normal | Normal | Yes | |
| | | | intron 81 | | | | splicing | Splicing | | |
| 22 | NEMALINE MYOPATHY 2 | NEB | NM_001271208.1 | +6 | BV-00035 | Class 1 | High confidence of normal | Normal | Yes | |
| | | | intron 51 | | | | splicing | Splicing | | |
| 23 | NEMALINE MYOPATHY 2 | NEB | NM_001271208.1 | +3 | BV-00037 | Class 4B | Very high risk of abnormal | Abnormal | Yes | Patient has hybrid NM/ |
| | | | intron 47 | | | | splicing | Splicing | | EHDS syndrome. Also has |
| | | | | | | | | | | PLOD1 variant. This |
| | | | | | | | | | | NEB variant could |
| | | | | | | | | | | explain nemaline rods |
| | | | | | | | | | | and myopathy. |
| 24 | NEMALINE MYOPATHY 2 | NEB | NM_001271208.1 | +1 | BV-00038 | Class 5 | High confidence extreme | Abnormal | Yes | Causative recessive |
| | | | intron 80 | | | | risk of abnormal splicing | Splicing | | mutation. AR NM. |
| 25 | NEMALINE MYOPATHY 2 | NEB | NM_001271208.1 | +1 | BV-00039 | Class 5 | High confidence extreme | Abnormal | Yes | Causative recessive |
| | | | intron 29 | | | | risk of abnormal splicing | Splicing | | mutation. AR NM. |
| 26 | NEMALINE MYOPATHY 2 | NEB | NM_001271208.1 | +5 | BV-00040 | Class 4B | Very high risk of abnormal | Abnormal | Yes | Causative recessive |
| | | | intron 45 | | | | splicing | Splicing | | mutation. AR NM. |
| 27 | NEMALINE MYOPATHY 2 | NEB | NM_001271208.1 | +1 | BV-00041 | Class 5 | High confidence extreme | Abnormal | | Causative recessive |
| | | | intron 25 | | | | risk of abnormal splicing | Splicing | | mutation. AR NM. |
| 28 | Microcephalic | PCNT | NM_006031.5 | −3 | BV-00042 | Class 2 | Normal splicing likely | Normal | Yes | ALAMUT programs predict |
| | osteodysplastic primordial | | exon 41 | | | | | Splicing | | abnormal splicing. Good |
| | dwarfism Type II | | | | | | | | | coverage, ~200 reads for |
| | | | | | | | | | | each patient. Clear |
| | | | | | | | | | | evidence for normal |
| | | | | | | | | | | splicing. |
| 29 | Atypical Gaucher disease, | PSAP | NM_001042465.2 | +5 | BV-00044 | Class 1 | High confidence of normal | Normal | Yes | |
| | Encephalopathy, Infantile | | intron 12 | | | | splicing | Splicing | | |
| | Krabbe disease, | | | | | | | | | |
| | Metachromatic | | | | | | | | | |
| | leukodystrophy. | | | | | | | | | |
| 30 | Autism suscpetibility, X- | RPL10 | NM_006013.4 | +3 | BV-00045 | Class 1 | High confidence of normal | Normal | Yes | |
| | linked intellectual | | intron 1 | | | | splicing | Splicing | | |
| | disability syndrome | | | | | | | | | |
| 31 | Blackfan-Diamond anemia | RPL5 | NM_000969.3 | +3 | BV-00046 | Class 3B | VUS; tangible risk of | 95% Normal | Yes | Low levels of intron |
| | | | intron 1 | | | | abnormal splicing | Splicing <5% | | retention. 36/961 reads. |
| | | | | | | | | abnormal | | All abnormal transcripts |
| | | | | | | | | splicing | | have the +3 variant. |
| 32 | Centronuclear myopathy, | RYR1 | NM_000540.2 | +3 | BV-00047 | Class 1 | High confidence of normal | Normal | Yes | |
| | Central Core disease, | | intron 37 | | | | splicing | Splicing | | |
| | Malignant hyperthermia of | | | | | | | | | |
| | anesthesia | | | | | | | | | |
| 33 | Centronuclear myopathy, | RYR1 | NM_000540.2 | +5 | BV-00048 | Class 1 | High confidence of normal | Normal | Yes | Turns rare into common |
| | Central Core disease, | | intron 48 | | | | splicing | Splicing | | donor. Check does not |
| | Malignant hyperthermia of | | | | | | | | | abnormally enhance splicing |
| | anesthesia | | | | | | | | | of a non-canonical exon |
| | | | | | | | | | | into transcript |
| | | | | | | | | | | to induce frameshift. |
| 34 | Centronuclear myopathy, | RYR1 | NM_000540.2 | +2 | BV-00049 | Class 5 | High confidence extreme | Abnormal | Yes | Causative mutation for this |
| | Central Core disease, | | intron 3 | | | | risk of abnormal splicing | Splicing | | patient. |
| | Congenital multicore | | | | | | | | | |
| | myopathy with external | | | | | | | | | |
| | ophthalmoplegia, | | | | | | | | | |
| | Malignant hyperthermia of | | | | | | | | | |
| | anesthesia | | | | | | | | | |
| 35 | COLE-CARPENTER | SEC24D | NM_014822.3 | −2 | BV-00051 | Class 1 | High confidence of normal | Normal | Yes | There is an alternative |
| | SYNDROME 1, Syndromic | | exon 5 | | | | splicing | Splicing | | acceptor being used |
| | osteogenesis imperfecta | | | | | | | | | downstream which can be |
| | | | | | | | | | | seen being used with and |
| | | | | | | | | | | without this variant. |
| 36 | AR Charcot Marie Tooth | SPG11 | NM_025137.3 | −2 | BV-00052 | Class 2 | Normal splicing likely | Normal | Yes | |
| | disease type 2X, AR spastic | | exon 16 | | | | | Splicing | | |
| | paraplegia type 11, | | | | | | | | | |
| | Juvenile amyotrophic | | | | | | | | | |
| | lateral sclerosis | | | | | | | | | |
| 37 | Early infantile epileptic | SZT2 | NM_015284.3 | +5 | BV-00054 | Class 2 | Normal splicing likely | Normal | Yes | |
| | encephalopathy | | intron 17 | | G > T | | | Splicing | | |
| 38 | Early infantile epileptic | SZT2 | NM_015284.3 | +5 | BV-00053 | Class 1 | High confidence of normal | Normal | Yes | |
| | encephalopathy | | intron 17 | | G > A | | splicing | Splicing | | |
| 39 | Combined oxidative | VARS2 | NM_020442.5 | −2 | BV-00057 | Class 3B | VUS; tangible risk of | Normal | Yes | Moderate coverage. ~50 |
| | phosphorylation defect | | exon 4 | | | | abnormal splicing | Splicing | | reads at each exon-exon |
| | type 20 | | | | | | | | | junction. Looks very normal. |
| | | | | | | | | | | Reads with and without the |
| | | | | | | | | | | SNP splice normally. |

Overall Predictive accuracy:
39/39 True Positive and True Negative predicted accurately
1/39 Marginal False positive call; CSP Predicted Class 4A; only low levels of abnormal splicing detected.

TABLE 4
Description of Clinical Splice Predictor
Variant Classification criteria.
Clinical Splice Predictor: Splice Prediction Classifications

| | |
|---|---|
| Class 1: | High confidence of normal splicing |
| Class 2: | Normal splicing likely |
| Class 3A: | Variant of uncertain significance; evidence |
| | consistent with normal splicing |
| Class 3B: | Variant of uncertain significance; evidence |
| | consistent with tangible risk of abnormal splicing |
| Class 4A: | High risk of abnormal splicing |
| Class 4B: | Very high risk of abnormal splicing |
| Class 5: | High confidence extreme risk of abnormal splicing |

### Criteria for Splice Prediction Classifications

### Class 1: High Confidence of Normal Splicing

### Criteria:

1. Variant may have an allele frequency in gnomAD that is inconsistent with: a) an autosomal dominant genetic disorder (mAF>0.001%) or b) an autosomal recessive genetic disorder (mAF>0.01%) or c) the number of observed homozygotes is inconsistent with a severe Mendelian disorder.
2. NIF: Variant splice site has all relevant windows where: a) VAR_{NIF} is maintained or increased, or b) NIF is greater than or equal to 50.
3. Previous Classifications: Multiple benign-only, or benign exceed pathogenic by 3-fold or more
4. Similar NIF-shift: Benign >>> Pathogenic. Benign classifications represent 90% or greater of all Similar NIF-shift variants.

### Class 2: Normal Splicing Likely

### Criteria:

1. Variant may have an allele frequency in gnomAD that is inconsistent with: a) an autosomal dominant genetic disorder (mAF>0.001%) or b) an autosomal recessive genetic disorder (mAF>0.01%) or c) the number of observed homozygotes is inconsistent with a severe Mendelian disorder.
2. NIF: Variant splice site has all relevant windows where: a) VAR_{NIF} is maintained or increased, or b) NIF is greater than or equal to 20.
3. Previous Classifications: Multiple benign-only, benign exceed pathogenic, or No Previous classifications with increase NIF in all relevant windows.
4. Similar NIF-shift: Benign >> Pathogenic. Benign classifications represent 75% or greater of all Similar NIF-shift variants.
   Class 3A: Variant of Uncertain Significance; Evidence Consistent with Normal Splicing

### Criteria:

1. NIF: Variant splice site has most relevant windows where: a) VAR_{NIF} is maintained or increased, or b) NIF is greater than or equal to 20.
2. Previous Classifications: No previous classifications, or benign-only, or benign=equal pathogenic, or benign exceed pathogenic.
3. Similar NIF-shift: Benign > Pathogenic.
   Class 3B: Variant of Uncertain Significance; Evidence Consistent with Tangible Risk of Abnormal Splicing

### Criteria:

1. Variant has an allele frequency in gnomAD that is consistent with a rare, severe Mendelian disorder.
2. NIF: Variant splice site has most relevant windows where VAR_{NIF} is decreased substantially
3. Previous Classifications: No previous classifications, or pathogenic-only, or pathogenic=equal pathogenic, or pathogenic exceed benign.
4. Similar NIF-shift: Pathogenic > Benign.

### Class 4A: High Risk of Abnormal Splicing

### Criteria:

1. Variant has an allele frequency in gnomAD that is consistent with a rare, severe Mendelian disorder.
2. NIF: Variant splice site has: a) at least one relevant windows where VAR_{NIF}=0, and/or, b) all relevant windows have a significant diminution in NIF count
3. Previous Classifications: a) Multiple pathogenic-only, b) Pathogenic exceed benign, or c) No previous classifications, with multiple windows of NIF=0.
4. Similar NIF-shift: Pathogenic >> Benign. Pathogenic classifications represent 90% or greater of all Similar NIF-shift variants.

### Class 4B: Very High Risk of Abnormal Splicing

### Criteria:

1. Variant has an allele frequency in gnomAD that is consistent with a rare, severe Mendelian disorder.
2. NIF: Variant splice site has: a) at least one relevant windows where VAR_{NIF}=0, and/or, b) all relevant windows have a significant diminution in NIF count with NIF<10
3. Previous Classifications: Consistent previous classifications as pathogenic across multiple windows of the variant splice site, where a) only pathogenic PC or b) pathogenic exceed benign by 3-fold or more in two or more windows of nine nucleotide.
4. Similar NIF-shift: Pathogenic >>> Benign. Pathogenic classifications represent 95% or greater of all Similar NIF-shift variants.

### Class 5: High Confidence Extreme Risk of Abnormal Splicing

### Criteria:

1. Variant has an allele frequency in gnomAD that is consistent with a rare, severe Mendelian disorder.
2. NIF: Variant splice site has three or four relevant windows where VAR_{NIF}=0
3. Previous Classifications: Multiple pathogenic-only, or pathogenic exceed benign by 3-fold or more in multiple windows.
5. Similar NIF-shift: Pathogenic >>> Benign. Pathogenic classifications represent 95% or greater of all Similar NIF-shift variants.

Appendix A. A list of Mendelian genes with clinically relevant phenotypes. This list has been filtered to exclude OMIM genes associated with traits and non-clinically relevant phenotypes such as eye colour, curly hair etc.

Appendix B. A compiled list of genes determined to induce developmental lethality with recessive knock-out in a murine mouse model via Mouse Genome Informatics (http://www.informatics.jax.org/downloads/reports/index.html) and the 8^{th} release of IMPC mouse phenotype data (ftp://ftp.ebi.ac.uk/pub/databases/impc/).

Appendix C. A compiled list of genes determined to induce human prenatal, perinatal or infantile lethality were derived from http://www.omim.org. OMIM phenotypic search terms were used to query text fields for terms associated with lethality before birth or shortly after birth.

### DETAILED DESCRIPTION OF EMBODIMENTS

Embodiments of the invention will now be described, by way of example only, with reference to the accompanying drawings.

In an embodiment related to the first embodiment, disclosed are methods of identifying an abnormal splice site in a sample splice site from a subject. Disclosed are methods relating to comparing a sample splice site from a subject with splice sites from a reference human genome sequence. The comparison comprises determining a measure of Native Intron Frequency of a splice site sequence from a subject relative to a reference human genome sequence, wherein Native Intron Frequency refers to a measure of the frequency of the splice site sequence from a subject in a reference human genome sequence. In certain embodiments, a measure of Native Intron Frequency refers to the number of times a splice site sequence from a subject appears in a reference human genome sequence. In certain embodiments, a measure of Native Intron Frequency refers to Percentile (NIF). In certain embodiments, the sample splice site from the subject is a donor splice site, a branch site, or an acceptor splice site. In certain embodiments, the sample splice site sequence comprises 4 to 12 nucleotides of a donor splice site. In certain embodiments, the sample splice site sequence comprises 4, 5, 6, 7, 8, 9, 10, 11 or 12 consecutive nucleotides of a donor splice site. In certain embodiments, the sample splice site sequence comprises 4 to 15 nucleotides of a donor splice site. In certain embodiments, the sample splice site sequence comprises 4, 5, 6, 7, 8, 9, 10, 11, 12, 13, 14 or up to 15 consecutive nucleotides of a donor splice site. In certain embodiments, the sample splice site sequence comprises 30 or more nucleotides of a donor splice site. In certain embodiments, the sample splice site sequence comprises 30 or more consecutive nucleotides of a donor splice site. In certain embodiments, the sample splice site sequence comprises 9 consecutive nucleotides of a donor splice site. In certain embodiments related to the first embodiment, the sample splice site is a donor splice site, and the method comprises more than one sample splice site sequence comprised in the same donor splice site, wherein each sample donor splice site sequence comprises 9 non-identical consecutive nucleotides of the donor splice site, and wherein the sample donor splice site sequences may comprise overlapping consecutive nucleotides of the donor splice site. In a related embodiment comprising at least six sample splice site sequences comprised in the same sample splice site, the sample splice site sequences correspond to at least nucleotide positions E⁻⁵ to D⁺⁴, E⁻⁴ to D⁺⁵, E⁻³ to D⁺⁶, E⁻² to D⁺⁷, E⁻¹ to D⁺⁸, and D⁺¹ to D⁺³ of a donor splice site. In a related embodiment comprising at least four sample splice site sequences comprised in the same sample splice site, the sample splice site sequences correspond to at least nucleotide positions E⁻⁴ to D⁺⁵, E⁻³ to D⁺⁶, E⁻² to D⁺⁷ and E⁻¹ to D⁺⁸ of a donor splice site.

In embodiments related to the first embodiment, the method of identifying an abnormal splice site in a sample splice site from a subject comprises (a) obtaining a first sample splice site sequence comprised in the sample splice site from the subject; and (b) determining a Native Intron Frequency of the first sample splice site sequence (NIFᵥₐᵣ₋₁); wherein an NIFᵥₐᵣ₋₁ of 0 indicates that the sample splice site is abnormal. In certain embodiments, the sample splice site from a subject is a donor splice site and the first sample donor splice site sequence comprises 9 consecutive nucleotides of the sample donor splice site. In certain embodiments, the sample splice site from a subject is a donor splice site and the method comprises determining a NIFᵥₐᵣ for more than one sample donor splice site sequence comprised in the same sample splice site, and the method of comprises (a) obtaining first and second sample donor splice site sequences; first, second, and third sample donor splice site sequences; first, second, third, and fourth sample donor splice site sequences; first, second, third, fourth, and fifth sample donor splice site sequences, or first, second, third, fourth, fifth, and sixth sample donor splice site sequences; wherein each sample donor splice site sequence is comprised in the sample donor splice site from the subject, wherein each sample donor splice site sequence comprises a non-identical set of 9 nucleotide positions of the sample donor splice site; and (b) determining a measure of Native Intron Frequency of the each sample donor splice site sequence; wherein a Native Intron Frequency of 0 (zero) for any sample donor splice site sequence indicates that the sample donor splice site is abnormal.

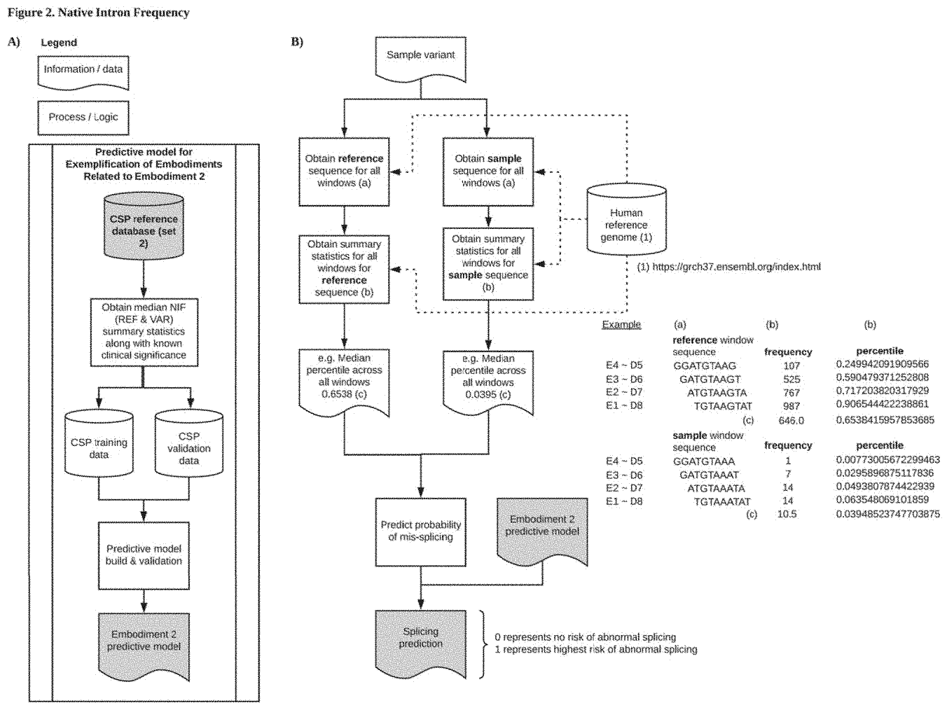

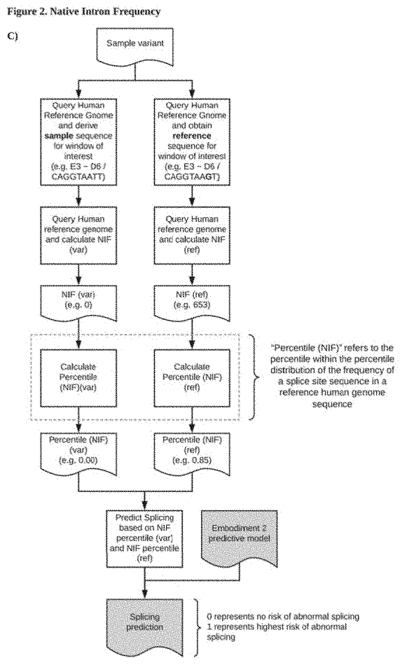

In an embodiment related to the second embodiment, methods of identifying an abnormal splice site in a sample splice site relate to comparing a measure of Native Intron Frequency of a sample splice site sequence with a measure of Native Intron Frequency of a reference splice site sequence, wherein the sample splice site sequence and reference splice site sequence originate from the same corresponding region of a gene. A change (or shift) in a measure of Native Intron Frequency of the sample splice site sequence in comparison to the Native Intron Frequency of a corresponding reference splice site sequence provides a measure of the risk of abnormal splicing for the sample splice site; the change (or shift) may be referred to herein as NIF-shift or shift in NIF for a sample splice site sequence. In certain embodiments, a measure of Native Intron Frequency of sample splice site sequence and a measure of Native Intron Frequency of a corresponding reference splice site sequence are determined, and a risk of abnormal splicing for the sample splice site is determined by comparing NIF-shift against a CSP reference database. In certain embodiments, a NIF-shift is determined for the sample splice site sequence from the measure of Native Intron Frequency of sample splice site sequence and a measure of Native Intron Frequency of a corresponding reference splice site sequence. NIF-shift may be determined by a ratiometric analysis of the measure of Native Intron Frequency of sample splice site sequence and the measure of Native Intron Frequency of a corresponding reference splice site sequence; or subtracting the measure of Native Intron Frequency of sample splice site sequence from the measure of Native Intron Frequency of a corresponding reference splice site sequence: or the like calculations. In certain embodiments, NIF-shift for the sample splice site is compared against a CSP reference database, wherein the CSP reference database comprises NIF-shift for variant splice sites clinically classified as abnormal splice sites or benign variant splice sites, and wherein the comparison comprises assessing a clinical classification(s) assigned to (a) variant splice site(s) having about the same NIF-shift as the sample splice site sequence. A risk of abnormal splicing may then be derived from the clinical classification(s) of each variant splice site having about the same NIF-shift as the sample splice site sequence. Given a CSP reference dataset comprising, e.g. NIF-shift with a known classification for each variant splice site, a machine learning or regression algorithm can be applied to calculate the risk of abnormal splicing for a sample splice site sequence. Given the input dataset, various techniques can be used to produce an indicator of the risk of abnormal splicing for the sample site sequence. Whilst a simple method is to apply a regression calculation to the data set to produce a regression equation, other techniques can be used. These can include applying support vector machines to the data set, and in the further alternative applying deep neural network learning techniques to the data set. In one embodiment, the risk of abnormal splicing is a number from 0 to 1, wherein 0 represents no risk of abnormal splicing and 1 represents highest risk of abnormal splicing. Exemplary embodiments related to the second embodiment are depicted in FIG. 2B.

In an embodiment related to the second embodiment, provided is a method of identifying an abnormal splice site in a sample splice site from a subject, said method comprising:
(a) obtaining a first sample splice site sequence comprised in the sample splice site from the subject;
(b) determining a measure of Native Intron Frequency of the first sample splice site sequence (NIFᵥₐᵣ₋₁);
(c) determining a Percentile (NIFᵥₐᵣ₋₁) of the first sample splice site sequence;
(d) determining a measure of Native Intron Frequency of a first reference splice site sequence (NIF_{ref-1}); wherein the first reference splice site sequence and the first sample splice site sequence each originate from the same corresponding region of a gene;
(e) determining a Percentile (NIF_{ref-1}) of the first reference splice site sequence; and
(f) determining a risk of abnormal splicing for the sample splice site by comparing the Percentile (NIFᵥₐᵣ₋₁) with the Percentile (NIF_{ref-1}) against a CSP reference database.

In embodiments related to the second embodiment, Percentile (NIFᵥₐᵣ₋₁) and Percentile (NIF_{ref-1}) are used in conjunction to infer the risk of abnormal splicing. In certain embodiments, a NIF-shift is determined for the sample splice site sequence from Percentile (NIFᵥₐᵣ₋₁) and Percentile (NIF_{ref-1}). NIF-shift may be determined by a ratiometric analysis of Percentile (NIFᵥₐᵣ₋₁) and Percentile (NIF_{ref-1}); or subtracting Percentile (NIFᵥₐᵣ₋₁) from Percentile (NIF_{ref-1}); or the like calculations. In certain embodiments, NIF-shift for the sample splice site sequence is compared against a CSP reference database, wherein the CSP reference database comprises NIF-shift for variant splice sites clinically classified as abnormal splice sites or benign variant splice sites, and wherein the comparison comprises assessing a clinical classification(s) assigned to (a) variant splice site(s) having about the same NIF-shift as the sample splice site sequence. A risk of abnormal splicing may then be derived from the clinical classification of each variant splice site with a clinical classification having about the same NIF-shift as the sample splice site sequence. Exemplary embodiments related to the second embodiment are depicted in FIG. 2B.

Given a dataset, e.g. a CSP reference database, comprising, e.g. a Percentile (NIFᵥₐᵣ), a Percentile (NIF_{ref}), and a known classification for each genetic variant, a machine learning or regression algorithm can be applied to calculate the risk of abnormal splicing for a sample splice site sequence. Given the input dataset, various techniques can be used to produce an indicator of the risk of abnormal splicing for the sample site sequence. Whilst a simple method is to apply a regression calculation to the data set to produce a regression equation, other techniques can be used. These can include applying support vector machines to the data set, and in the further alternative applying deep neural network learning techniques to the data set.

It will be understood that in any embodiments comprising Percentile (NIF), a measure of NIF (eg NIF or NIF (count) may be used instead.

An exemplary machine learning dataset suitable for embodiments related to any embodiment described herein, may comprise one or more datasets related to non-identical nucleotide positions of a sample splice site as shown below. It will be appreciated that the number of sample splice site sequences from the same sample splice site may vary in total nucleotide composition and nucleotide position with respect to the sample splice site.

| | | | | | | | Machine |
|---|---|---|---|---|---|---|---|
| | | | | | | | Learning |
| | E⁻⁵~D⁺⁴ | E⁻⁴~D⁺⁵ | E⁻³~D⁺⁶ | E⁻²~D⁺⁷ | E⁻¹~D⁺⁸ | D⁺¹~D⁺⁹ | dataset |
| −5 | X | | | | | | 1 |
| −4 | X | X | | | | | 2 |
| −3 | X | X | X | | | | 3 |
| −2 | X | X | X | X | | | 4 |
| −1 | X | X | X | X | X | | 5 |
| 1 | X | X | X | X | X | X | 6 |
| 2 | X | X | X | X | X | X | 6 |
| 3 | X | X | X | X | X | X | 6 |
| 4 | X | X | X | X | X | X | 6 |
| 5 | | X | X | X | X | X | 7 |
| 6 | | | X | X | X | X | 8 |
| 7 | | | | X | X | X | 9 |
| 8 | | | | | X | X | 10 |
| 9 | | | | | | X | 11 |

In the above exemplary table, the first column indicates the nucleotide position of a sample splice site in which a variation from a corresponding reference splice site sequence occurs. For example, for a sample splice site variant that resides in the −1 position of a donor splice site, a NIFᵥₐᵣ and corresponding NIF_{ref} (and/or a Percentile (NIFᵥₐᵣ) and corresponding Percentile (NIF_{ref})) for sample splice site sequences corresponding to nucleotide position E⁻⁵˜D⁺⁴ through to E⁻¹˜D⁺⁵ of the sample donor splice site may be analysed, and so on.

In certain embodiments related to the second embodiment, the sample splice site may be a donor splice site and the donor splice site sequence comprises 4 to 12 nucleotides of the sample donor splice site. In certain embodiments related to the second embodiment, the sample splice site is a donor splice site and the donor splice site sequence comprises 4, 5, 6, 7, 8, 9, 10, 11, or 12 consecutive nucleotides of the sample donor splice site. In certain embodiments related to the second embodiment, the sample splice site is a donor splice site and the donor splice site sequence comprises 4 to 15 nucleotides of a donor splice site. In certain embodiments related to the second embodiment, the sample splice site sequence comprises 4, 5, 6, 7, 8, 9, 10, 11, 12, 13, 14 or up to 15 consecutive nucleotides of a donor splice site. In certain embodiments related to the second embodiment, the sample splice site sequence comprises 30 or more nucleotides of a donor splice site. In certain embodiments, the sample splice site sequence comprises 30 or more consecutive nucleotides of a donor splice site. In certain embodiments related to the second embodiment, the sample splice site is a donor splice site and the donor splice site sequence comprises 9 consecutive nucleotides of the sample donor splice site. In further embodiments related to the second embodiment, the sample splice site from a subject is a donor splice site and the method comprises analysing more than one donor splice site sequence comprised in the same sample donor splice site, wherein said method comprises, for example, obtaining first and second sample donor splice site sequences; first, second, and third sample donor splice site sequences; first, second, third, and fourth sample donor splice site sequences; first, second, third, fourth, and fifth sample donor splice site sequences; first, second, third, further, fifth, and sixth sample donor splice site sequence, and so on; wherein each sample donor splice site sequence is comprised in the sample donor splice site from the subject. Each Percentile (NIFᵥₐᵣ₋₁) and corresponding Percentile (NIF_{ref-1}) are used in conjunction, e.g. by calculating a respective NIF-shift, against a CSP reference database to infer the risk of abnormal splicing. A risk of abnormal splicing may then be derived from the clinical classification of each variant splice site with a clinical classification having about the same NIF-shift as the sample splice site sequences. An increasing number of sample splice site sequences characterised as abnormal, increases the risk of abnormal splicing.

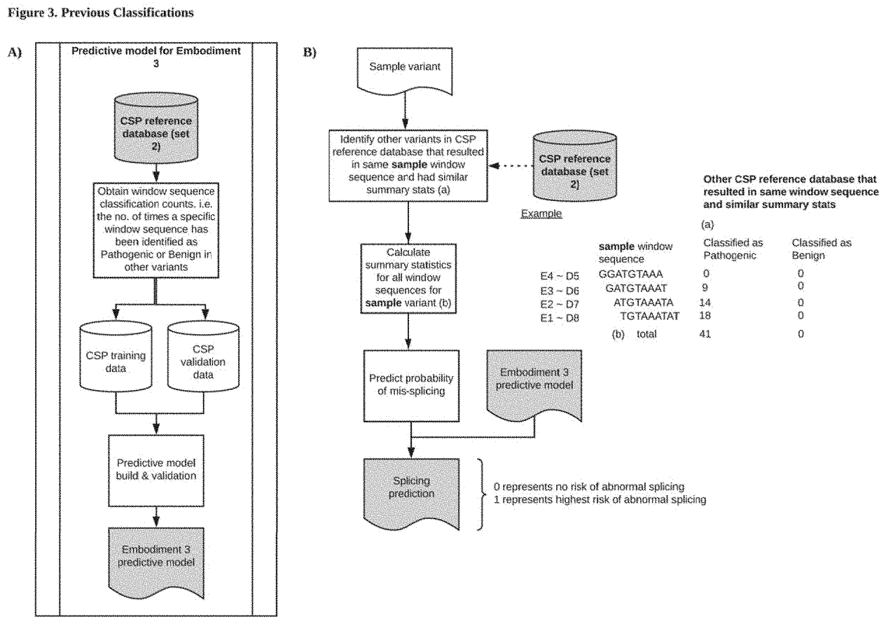

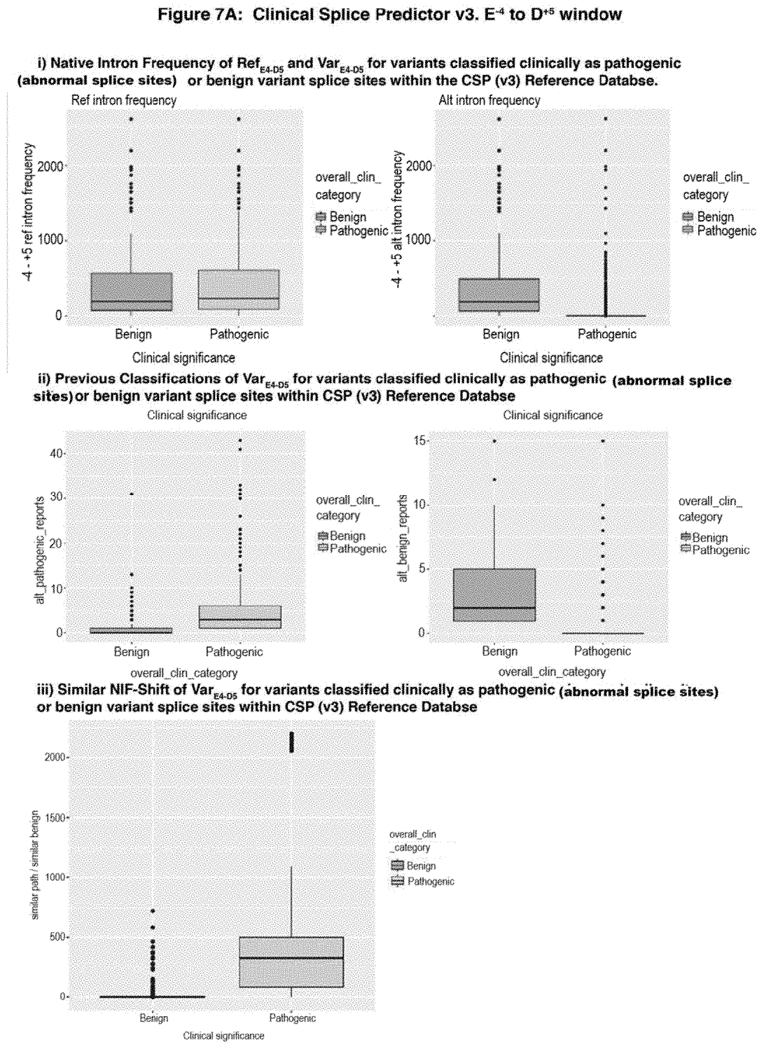

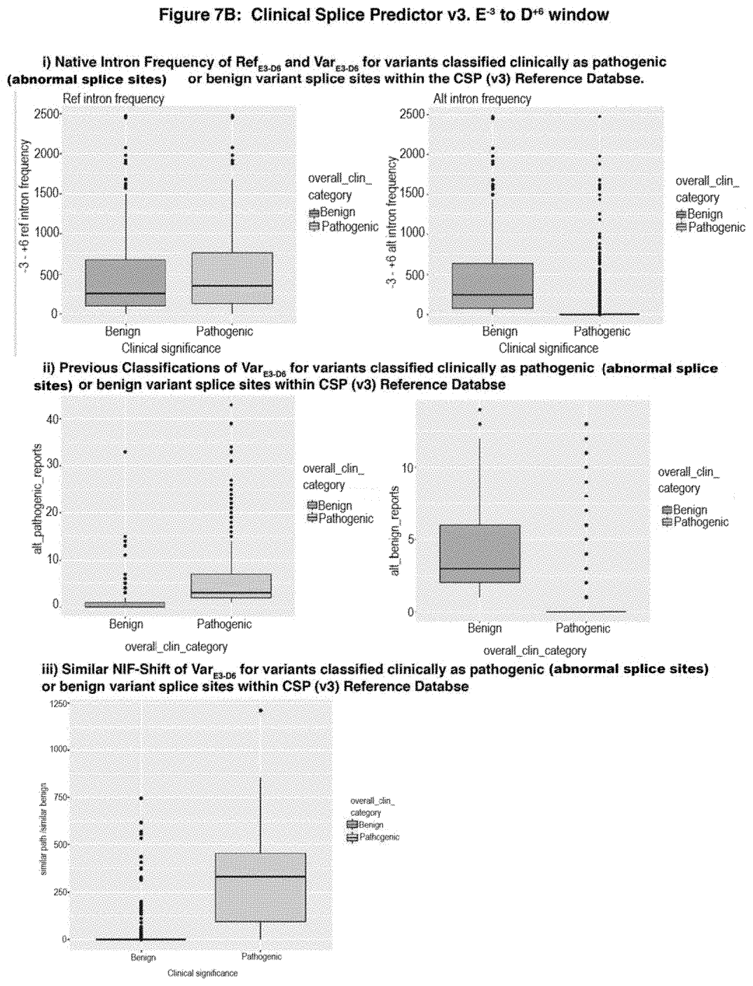

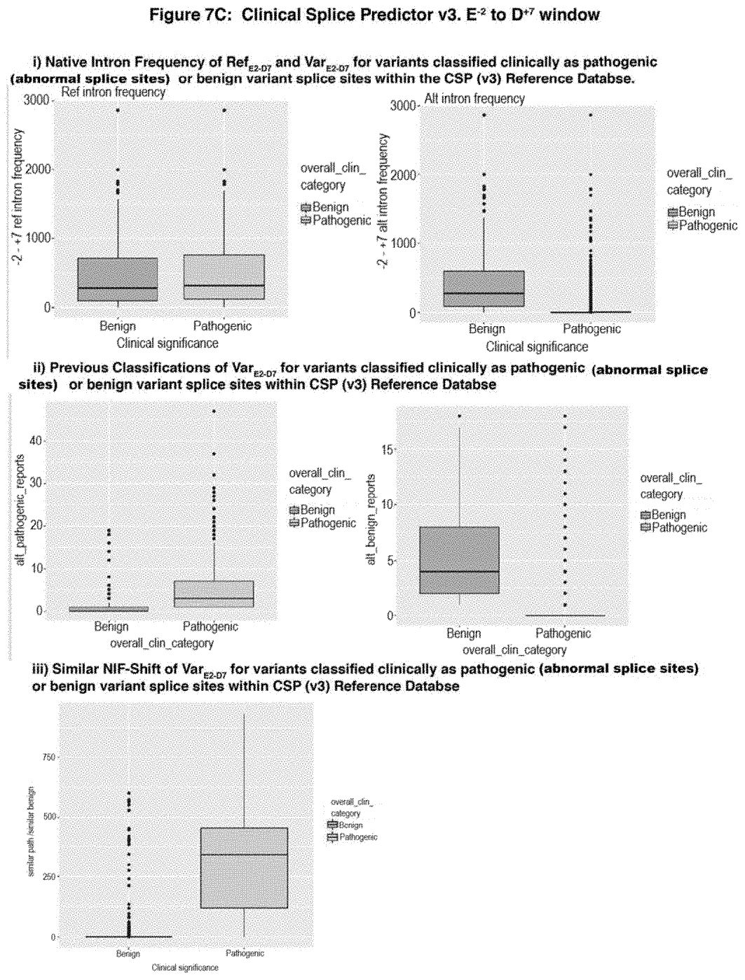

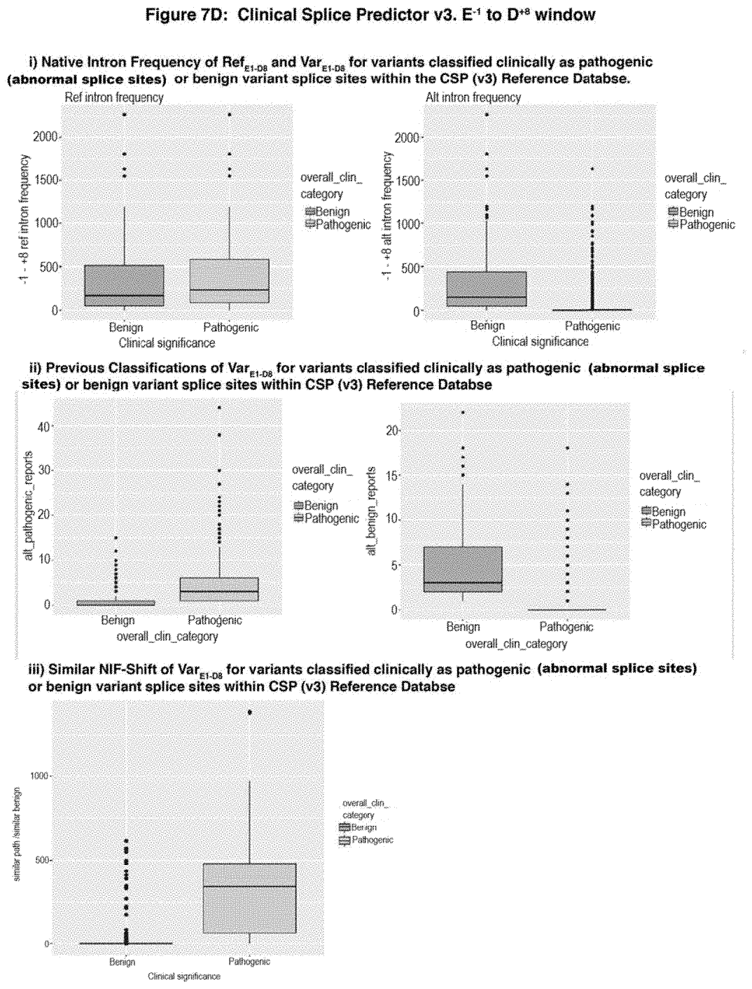

In an embodiment related to the third embodiment, provided are methods of identifying an abnormal splice site in a sample splice site from a subject related to comparing the clinical classification(s) of the nucleotide sequence of a sample splice site sequence in relation to any variant splice site comprising the same nucleotide sequence. The method comprises assessing the clinical classification(s), if available, of each appearance of a nucleotide sequence of a sample splice site sequence in any variant splice site in any gene, e.g. a splice site comprised in the same gene as the sample splice site but at another intron/exon location; a splice site comprised in a gene different from the gene comprising the sample splice site, and so on. In certain embodiments, the method further comprises assessing the clinical classification(s), if available, of each appearance of the nucleotide sequence of the reference splice site in any variant splice site in any gene. Collections of variant genes and/or variant splice sites relating to a disorder with an associated clinical classification, including for example, pathogenic, likely pathogenic, likely benign, likely benign, are available, including for example the collections available as ClinVar, HGMD, etc. A nucleotide sequence comprised in a sample splice site from a subject and/or a nucleotide sequence comprised in a corresponding reference splice site can be searched in such a collection for its appearance and the associated clinical classification of each appearance of the searched nucleotide sequence can be determined. In certain embodiments, a CSP reference database comprises variant wherein a variant clinically classified as “pathogenic” or “likely pathogenic” is assigned as an “abnormal splice site” and a variant clinically classified as “benign” or “likely benign” is assigned as a “benign variant splice site”. It will be appreciated that the same nucleotide sequence may be classified as an abnormal splice site in the context of one variant splice site comprised in a CSP database and may be classified as a benign variant splice site in the context of a different variant splice site comprised in the CSP database. A CSP reference database may comprise variants affecting only a donor splice site, including exonic variants that are non-code changing variants (synonymous exonic variants). For example, part ii of each of FIG. 7A to 7D shows that for a 9 nucleotide donor splice site sequence classified as a benign variant splice site (“benign”), there are multiple reports for this 9 nucleotide sequence as a benign variant splice site in donor splice sites of different genes (and different exon/introns) and, conversely, reports of this 9 nucleotide sequence as an abnormal splice site (“pathogenic”) are rare. Likewise, part ii of each of FIG. 7A to 7D show that that for a 9 nucleotide donor splice site sequence classified as an abnormal splice site (“pathogenic”), there are multiple reports for this 9 nucleotide sequence as an abnormal splice site (“pathogenic) in donor splice sites of different genes (and different exon/introns) and, conversely, reports of this 9 nucleotide sequence as a benign variant splice site (“benign”) are rare. An exemplary embodiment related to the third embodiment is depicted in FIG. 3.

In an embodiment related to the third embodiment, the method of identifying an abnormal splice site in a sample splice site from a subject, said method comprises:

(a) obtaining a first sample splice site sequence comprised in the sample splice site from the subject;
(b) determining a clinical classification(s) associated with the nucleotide sequence of the first sample splice site sequence;
(c) determining a risk of abnormal splicing for the sample splice site by assessing the clinical classification(s) of the nucleotide sequence of the first sample splice site sequence determined in step (b).

In an embodiment related to the third embodiment, the method of identifying an abnormal splice site in a sample splice site from a subject, said method comprises:

(a) obtaining a first sample splice site sequence comprised in the sample splice site from the subject;
(b) obtaining a first reference splice site sequence; wherein the first reference splice site sequence and the first sample splice site sequence each originate from the same corresponding region of a gene;
(c) determining a clinical classification(s) associated with the nucleotide sequence of the first sample splice site sequence;
(d) determining a clinical classification(s) associated with the nucleotide sequence of the first reference splice site sequence; and
(e) determining a risk of abnormal splicing for the sample splice site by assessing the clinical classification(s) of the nucleotide sequence of the first sample splice site sequence determined in step (c) and the clinical classification(s) of the nucleotide sequence of the first reference splice site sequence determined in step (d).

In embodiments related to the third embodiment, clinical classification(s) of a nucleotide sequence of a splice site sequence (eg, sample splice site sequence, reference splice site sequence) may be determined from a data base comprising known genetic variants with an associated clinical classification (eg, abnormal splice site, benign variant splice site). A clinical classification of a nucleotide sequence of a splice site sequence may be determined from a CSP reference database, wherein the CSP reference database comprises nucleotide sequences of variant splice sites with corresponding clinical classifications (eg, abnormal splice site, benign variant splice site).

In certain embodiments related to the third embodiment, the sample splice site may be a donor splice site and the donor splice site sequence may comprise 4 to 12 nucleotides of the sample donor splice site. In certain embodiments related to the third embodiment, the sample splice site is a donor splice site and the donor splice site sequence comprises 4, 5, 6, 7, 8, 9, 10, 11, or 12 consecutive nucleotides of the sample donor splice site. In certain embodiments related to the third embodiment, the sample splice site is a donor splice site and the donor splice site sequence comprises 4 to 15 nucleotides of a donor splice site. In certain embodiments related to the third embodiment, the sample splice site sequence comprises 4, 5, 6, 7, 8, 9, 10, 11, 12, 13, 14 or up to 15 consecutive nucleotides of a donor splice site. In certain embodiments related to the third embodiment, the sample splice site sequence comprises 30 or more nucleotides of a donor splice site. In certain embodiments, the sample splice site sequence comprises 30 or more consecutive nucleotides of a donor splice site. In certain embodiments related to the third embodiment, the sample splice site is a donor splice site and the donor splice site sequence comprises 9 consecutive nucleotides of the sample donor splice site. In further embodiments related to the third embodiment, the sample splice site from a subject is a donor splice site and the method comprises analysing more than one donor splice site sequences comprised in the same sample donor splice site, wherein said method comprises, for example, obtaining first and second sample donor splice site sequences; first, second, and third sample donor splice site sequences; first, second, third, and fourth sample donor splice site sequences; first, second, third, fourth, and fifth sample donor splice site sequences; first, second, third, fourth, fifth, and sixth sample donor splice site sequences, and so on; wherein each sample donor splice site sequence is comprised in the sample donor splice site from the subject. A clinical classification(s) associated with the nucleotide sequence of each sample splice site sequence is determined and, optionally, a clinical classification(s) associated with the nucleotide sequence of each corresponding reference splice site sequence is determined.

Embodiments related to the third embodiment, a risk of abnormal splicing for a sample splice site may be determined by assessing the clinical classifications associated with the nucleotide sequence(s) of one or more sample splice site sequences comprised in a sample splice site. The risk of abnormal splicing increases with increasing instances of abnormal splice sites comprising the nucleotide sequence of a sample splice site sequence, e.g. the number of variant splice sites comprised in a CSP reference database, wherein the variant splice site comprises the nucleotide sequence of the sample splice site sequence, and wherein the variant splice site is clinically classified as an abnormal splice site. A risk of abnormal splicing may be assigned a value from 0 to 1, wherein 0 represents no risk of abnormal splicing and 1 represents highest risk of abnormal splicing. In embodiments comprising more than one sample splice site sequence, a risk of abnormal splicing comprises analysing the clinical classification(s) of the nucleotide sequences corresponding to each sample splice site sequence.

For example, in a method of the third embodiment, wherein the sample splice site is a donor splice site, the sample donor splice site sequence comprises 9 consecutive nucleotide of the donor splice site, and the method is repeated with six non-identical donor splice site sequences comprised in the same sample splice site (E⁻⁵ to D⁺⁴, E⁻⁴ to D⁺⁵, E⁻³ to D⁺⁶, E⁻² to D⁺⁷, E⁻¹ to D⁺⁸, and D⁺¹ to D⁺⁹) it is possible to create a series of 11 data sets, as follows:

| | | | | | | | Machine |
|---|---|---|---|---|---|---|---|
| | | | | | | | Learning |
| | E⁻⁵~D⁺⁴ | E⁻⁴~D⁺⁵ | E⁻³~D⁺⁶ | E⁻²~D⁺⁷ | E⁻¹~D⁺⁸ | D⁺¹~D⁺⁹ | dataset |
| −5 | X | | | | | | 1 |
| −4 | X | X | | | | | 2 |
| −3 | X | X | X | | | | 3 |
| −2 | X | X | X | X | | | 4 |
| −1 | X | X | X | X | X | | 5 |
| 1 | X | X | X | X | X | X | 6 |
| 2 | X | X | X | X | X | X | 6 |
| 3 | X | X | X | X | X | X | 6 |
| 4 | X | X | X | X | X | X | 6 |
| 5 | | X | X | X | X | X | 7 |
| 6 | | | X | X | X | X | 8 |
| 7 | | | | X | X | X | 9 |
| 8 | | | | | X | X | 10 |
| 9 | | | | | | X | 11 |

A machine learning set is thus comprised of 11 data sets. Each dataset is specialised at summarizing the patterns of abnormal splicing site/benign variant splice site that occurs within that window. The number of abnormal splicing site/benign variant splice site are used to infer the risk of abnormal splicing of a splice site. The dataset is then used as the foundation for regression or machine learning to calculate the risk of abnormal splicing for a sample splice site from a subject. Given the input dataset, various techniques can be used to produce an indicator of the risk of abnormal splicing for the sample site sequence. Whilst a simple method is to apply a regression calculation to the data set to produce a regression equation, other techniques can be used. These can include applying support vector machines to the data set, and in the further alternative applying deep neural network learning techniques to the data set.

It will be understood that in a method related to the third embodiment, alternative compilations of data may be used to create a machine learning dataset. For example, an alternative approach with regard to the E⁻⁵ to D⁺⁹ donor sample site and having six unique donor sample site sequence each with 9 consecutive nucleotides of the donor sample site can be applied as follows:

| | | | | | | | Machine |
|---|---|---|---|---|---|---|---|
| | | | | | | | Learning |
| | E⁻⁵~D⁺⁴ | E⁻⁵~D⁺⁵ | E⁻⁵~D⁺⁶ | E⁻⁵~D⁺⁷ | E⁻⁵~D⁺⁸ | D⁺⁵~D⁺⁹ | dataset |
| −5 | X | X | X | X | X | X | 1 |
| −4 | X | X | X | X | X | X | 1 |
| −3 | X | X | X | X | X | X | 1 |
| −2 | X | X | X | X | X | X | 1 |
| −1 | X | X | X | X | X | X | 1 |
| | | | | | | | Machine |
| | | | | | | | Learning |
| | E⁻⁵~D⁺⁴ | E⁻⁴~D⁺⁵ | E⁻³~D⁺⁶ | E⁻²~D⁺⁷ | E⁻¹~D⁺⁸ | D⁺¹~D⁺⁹ | dataset |
| 1 | X | X | X | X | X | X | 2 |
| 2 | X | X | X | X | X | X | 2 |
| 3 | X | X | X | X | X | X | 2 |
| 4 | X | X | X | X | X | X | 2 |
| 5 | X | X | X | X | X | X | 3 |
| 6 | X | X | X | X | X | X | 3 |
| 7 | X | X | X | X | X | X | 3 |
| 8 | X | X | X | X | X | X | 3 |
| 9 | X | X | X | X | X | X | 3 |

Again, the data set can be utilised as an input to standard machine learning techniques to provide for a descriptive output of a subsequent test subject.

In an embodiment related to the fourth embodiment, methods of identifying an abnormal splice site in a sample splice site from a subject relate to assessing the clinical classification of a splice site determined to be similar to a sample splice site from the subject. In one embodiment, a splice site is determined to be similar to a sample splice site from the subject by determining a relative shift in NIF (NIF-shift) of a sample splice site sequence, calculating a range of values around the NIF-shift of the sample splice site sequence, and querying a database comprising NIF-shift for variant splice sites and corresponding clinical classifications (eg abnormal splice site or benign variant splice site) for variants splice sites having a NIF-shift within the calculated range of NIF-shift for the sample splice site sequence. Variant splice sites identified as having NIF-shift within the calculated range of NIF-shift for the sample splice site sequence may be referred to as “similar NIF-shift variants”. A risk of abnormal splicing may be determined by analysing the clinical classification of similar NIF-shift variants. The risk of abnormal splicing increases with increasing instances of similar NIF-shift variants that are clinically classified as abnormal splice sites, e.g. the number of variant splice sites comprised in a CSP reference database, wherein the variant splice site has an NIF-shift within the range of NIF-shift for the sample splice site, and wherein the variant splice site is clinically classified as an abnormal splice site. A risk of abnormal splicing may be assigned a value from 0 to 1, wherein 0 represents no risk of abnormal splicing and 1 represents highest risk of abnormal splicing. It will be appreciated that for embodiments comprising more than one sample splice site sequence from the sample sample splice site, a risk of abnormal splicing is considered from all similar NIF-shift variants with respect to each range of NIF-shift for each sample splice site sequence.

An embodiment related to the fourth embodiment is a method of identifying an abnormal splice site in a sample splice site from a subject, said method comprising:

(a) obtaining a first sample splice site sequence comprised in the sample splice site from the subject;
(b) determining a measure of Native Intron Frequency of the first sample splice site sequence (NIFᵥₐᵣ₋₁);
(c) determining a Percentile (NIFᵥₐᵣ₋₁) of the first sample splice site sequence;
(d) determining a measure of Native Intron Frequency of a first reference splice site sequence (NIF_{ref-1}); wherein the first reference splice site sequence and the first sample splice site sequence originate from the same corresponding region of a gene;
(e) determining a Percentile (NIF_{ref-1}) of the first reference splice site sequence;
(f) calculating a lower and an upper bound for Percentile (NIFᵥₐᵣ₋₁) and calculating a lower and an upper bound for Percentile (NIF_{ref-1});
(g) determining a range of NIF-shift by comparing the lower and upper bounds for Percentile (NIFᵥₐᵣ₋₁) with the lower and upper bounds for Percentile (NIF_{ref-1}) calculated in (f);
(h) identifying (a) similar NIF-shift variant(s), wherein a similar NIF-shift variant refers to a splice site sequence with a NIF-shift within the range of NIF-shift determined in (g);
(i) determining (a) clinical classification(s) associated with each similar NIF-shift variant identified in step (h); and
(j) determining a risk of abnormal splicing for the sample splice site by assessing the clinical classification determined in step (i) for each similar NIF-shift variant identified in step (h).

In embodiments related to the fourth embodiment, the sample splice site is a donor splice site, steps (a) to (i) are repeated with up to five sample splice site sequences and corresponding respective reference splice site sequences, and step (j) includes assessing the clinical classification associated with each similar NIF-shift variant identified in each step (h).

In embodiments related to the fourth embodiment, Percentile (NIFᵥₐᵣ₋ₓ) and Percentile (NIF_{ref-x}) may be used in combination to determine a measure of NIF-shift and a range of NIF-shift may be calculated. In one embodiment, a range of NIF-shift of the sample splice site sequence is compared to a dataset comprising variant splice sites with known clinical classification (eg, abnormal splice site or benign variant splice site) and a corresponding NIF-shift is determined from a combination of Percentile (NIFᵥₐᵣ) and a corresponding Percentile (NIF_{ref}) for each variant splice site included in the dataset. In embodiments related to the fourth embodiment, NIFᵥₐᵣ₋ₓ and NIF_{ref-x} may be used in combination to determine a measure of NIF-shift and a range of NIF-shift may be calculated. In one embodiment, a range of NIF-shift of the sample splice site sequence is compared to a dataset comprising genetic variants of splice sites with known clinical classification (eg, abnormal splice site or benign variant splice site) and a corresponding NIF-shift is determined from a combination of NIFᵥₐᵣ and a corresponding NIF_{ref} for each genetic variant included in the dataset. Given a dataset comprising NIF-shift and a known classification for each variant splice site included in the dataset, a machine learning or regression algorithm can be applied to identify genetic variants comprised in the dataset that are similar to the sample splice site of the subject.

An embodiment related to the fourth embodiment is a method of identifying an abnormal splice site in a sample splice site from a subject, said method comprising:

(a) obtaining a first sample splice site sequence comprised in the sample splice site from the subject;
(b) determining a measure of Native Intron Frequency of the first sample splice site sequence (NIFᵥₐᵣ₋₁);
(c) determining a measure of Native Intron Frequency of a first reference splice site sequence (NIF_{ref-1}); wherein the first reference splice site sequence and the first sample splice site sequence originate from the same corresponding region of a gene;
(d) calculating a lower and an upper bound for NIFᵥₐᵣ₋₁ and calculating a lower and an upper bound for NIF_{ref-1};
(e) determining a range of NIF-shift by comparing the lower and upper bounds for NIFᵥₐᵣ₋₁ with the lower and upper bounds for NIF_{ref-1} calculated in (d);
(f) identifying (a) similar NIF-shift variants, wherein a similar NIF-shift variant refers to a splice site sequence with a NIF-shift within the range of NIF-shift determined in (e);
(g) determining a clinical classification associated with each similar NIF-shift variant identified in step (f); and
(h) determining the risk of abnormal splicing for the sample splice site by assessing the clinical classification determined in step (g) for each similar NIF-shift variant identified in step (f).

In embodiments related to the fourth embodiment, identification of similarity is based on a comparison of relative shift in NIF, which is a measure of the shift in NIF of a reference splice site sequence in comparison to NIF of a variant splice site sequence. The determination of similarity is independent of nucleotide sequence. A variant splice site sequence comprised in a dataset with a clinical classification (eg, abnormal splice site or benign variant splice site) and a corresponding NIF-shift may be identified as similar to a sample splice site sequence when the NIF-shift of the variant splice site sequence falls within a range of NIF-shift values centred about a NIF-shift of the sample splice site sequence.

A range of NIF-shift for a sample splice site sequence may be calculated by

(a) determining a measure of Native Intron Frequency of a sample splice site sequence, eg, NIFᵥₐᵣ₋ₓ or Percentile (NIFᵥₐᵣ₋ₓ), and determining a measure of Native Intron Frequency of a corresponding reference splice site sequence, e.g. NIF_{ref-x} or Percentile (NIF_{ref-x}); wherein the reference splice site sequence and the sample splice site sequence each originate from the same corresponding region of a gene;
(b) determining an upper and a lower bound for each measure recited in step (a), e.g. NIFᵥₐᵣ₋ₓ and NIF_{ref-x}, wherein NIFᵥₐᵣ₋ₓ lower bound is (e^{((log(NIFvar))*(1-NIF_shift percentage))}), NIFᵥₐᵣ₋ₓ upper bound is (e^{((log(NIFvar))*(1+NIF_shift percentage))}), NIF_{ref-x} lower bound is (e^{((log(NIFref))*(1-NIF_shift percentage))}), NIF_{ref-x} upper bound is (e^{((log(NIFref))*(1+NIF_shift percentage))})_{f};
wherein the respective upper and lower bounds provide a range of NIF-shift for a sample splice site sequence. NIF-shift percentage may be about 2%, about 2.5%, about 5%, or about 10%. A machine learning dataset may be created comprising a NIF shift for each variant splice site with a clinical classification (eg, abnormal splice site or benign variant splice site). This dataset may be used for regression or machine learning to calculate the risk of abnormal splicing for a sample splice site on the basis of a range of NIF-shift of a sample splice site sequence.

In further embodiments related to the fourth embodiment, the sample splice site may be a donor splice site. In certain embodiments, the sample splice site sequence comprises 4 to 12 nucleotides of a donor splice site. In certain embodiments, the sample splice site sequence comprises 4, 5, 6, 7, 8, 9, 10, 11, or 12 consecutive nucleotides of a donor splice site. In certain embodiments related to the fourth embodiment, the sample splice site is a donor splice site and the donor splice site sequence comprises 4 to 15 nucleotides of a donor splice site. In certain embodiments related to the third embodiment, the sample splice site sequence comprises 4, 5, 6, 7, 8, 9, 10, 11, 12, 13, 14 or up to 15 consecutive nucleotides of a donor splice site. In certain embodiments related to the third embodiment, the sample splice site sequence comprises 30 or more nucleotides of a donor splice site. In certain embodiments, the sample splice site sequence comprises 30 or more consecutive nucleotides of a donor splice site. In certain embodiments, the sample splice site sequence comprises 9 consecutive nucleotides of a donor splice site.

Methods of identifying an abnormal splice site in a sample splice site further relate to combinations of any method or any embodiment herein disclosed, including combinations of embodiments related to the first, second, and third embodiments or embodiments related to the first, second and fourth embodiments. Combinations of embodiments related to the first, second, third, and/or fourth embodiments are envisioned. Combinations of embodiments related to the second, third, and fourth embodiments are envisioned. Combinations of embodiments related to the second and fourth embodiments are envisioned.

In an embodiment related to the fifth embodiment, provided is a method of identifying an abnormal splice site in a sample splice site from a subject, said method comprising

(a) obtaining a first sample splice site sequence comprised in the sample splice site from the subject;
(b) determining a measure of Native Intron Frequency of the first sample splice site sequence (NIFᵥₐᵣ₋₁);
(c) determining a Percentile (NIFᵥₐᵣ₋₁) of the first sample splice site sequence;
(d) determining a measure of Native Intron Frequency of a first reference splice site sequence (NIF_{ref-1}); wherein the first reference splice site sequence and the first sample splice site sequence each originate from the same corresponding region of a gene;
(e) determining a Percentile (NIF_{ref-1}) of the first reference splice site sequence;
(f) determining a clinical classification(s) associated with the nucleotide sequence of the first sample splice site sequence;
(g) optionally determining a clinical classification(s) associated with the nucleotide sequence of the first reference splice site sequence;
(h) calculating a lower and an upper bound for Percentile (NIFᵥₐᵣ₋₁) and calculating a lower and an upper bound for Percentile (NIF_{ref-1});
(i) determining a range of NIF-shift by comparing the lower and upper bounds for Percentile (NIFᵥₐᵣ₋₁) with the lower and upper bounds for Percentile (NIF_{ref-1}) calculated in (h);
(j) identifying (a) similar NIF-shift variant(s), wherein a similar NIF-shift variant refers to a splice site sequence with a NIF-shift within the range of NIF-shift determined in (i);
(k) determining (a) clinical classification(s) associated with each similar NIF-shift variant identified in step (j); and
(l) determining the risk of abnormal splicing for the sample splice site by (1) comparing the Percentile (NIFᵥₐᵣ₋₁) with the Percentile (NIF_{ref-1}) against a CSP reference database, (2) assessing the clinical classification(s) associated with the nucleotide sequence of the first sample splice site sequence determined in step (f); and (3) assessing the clinical classification determined in step
(k) for each similar NIF-shift variant identified in step (j).

In certain embodiments, the sample splice site is a donor splice site, steps (a) to (l) are repeated with up to five sample splice site sequences and corresponding respective reference splice site sequences, and step (l) includes assessing (1) for all sample splice site sequences, (2) for all sample splice site sequences, and (3) for all sample splice site sequences.

Machine learning and dataset analysis of step (l) may be performed in accordance with the second, third, and fourth embodiments.

In a related embodiment, step (g) is carried out; and step (l) may further comprise as part of (2), analysing the clinical classification(s) associated with the nucleotide sequence of the first reference splice site sequence determined in step (g). Embodiments may comprise determining a risk of abnormal splicing expressed as a number from 0 to 1 for each of (1), (2), and (3) comprised in step (l), wherein 0 represents no risk of abnormal splicing and 1 represents highest risk of abnormal splicing.

In further embodiments related to the fifth embodiment, the sample splice site is a donor splice site. In certain embodiments, the sample splice site sequence comprises 4 to 12 nucleotides of a donor splice site. In certain embodiments, the sample splice site sequence comprises 4, 5, 6, 7, 8, 9, 10, 11, or 12 consecutive nucleotides of a donor splice site. In certain embodiments related to the fifth embodiment, the sample splice site is a donor splice site and the donor splice site sequence comprises 4 to 15 nucleotides of a donor splice site. In certain embodiments related to the fifth embodiment, the sample splice site sequence comprises 4, 5, 6, 7, 8, 9, 10, 11, 12, 13, 14 or up to 15 consecutive nucleotides of a donor splice site. In certain embodiments related to the fifth embodiment, the sample splice site sequence comprises 30 or more nucleotides of a donor splice site. In certain embodiments, the sample splice site sequence comprises 30 or more consecutive nucleotides of a donor splice site. In certain embodiments, the sample splice site sequence comprises 9 consecutive nucleotides of a donor splice site.

Also provided in further embodiments of any of the embodiments provide herein are methods of diagnosing a subject with a known genetic disorder or cancer wherein the sample splice site originates from a gene associated with known Mendelian disorder or cancer. In the methods herein disclosed, a sample splice site obtained from the subject may be a splice site from a predetermined gene associated with known genetic disorder or cancer. Thereby identification of an abnormal splice site in a sample splice site from a subject indicates a diagnosis of a genetic disease or cancer in the subject.

Also provided in further embodiments of any of the embodiments provided herein are methods relating to providing genetic testing services, including providing a risk of abnormal splicing of a sample splice site, to an individual. In one embodiment, provided is a method of providing to an individual a risk of abnormal splicing of a sample splice site from a subject, which is directly accessible by said individual through a computer interface, said method comprising

(a) providing a mechanism for said individual to input at least one sample splice site from a subject;
(b) determining a risk of abnormal splicing of a sample splice site sequence from a subject by
(i) obtaining a first sample splice site sequence comprised in the sample splice site from the subject input by said individual; and
(ii) determining a measure of Native Intron Frequency of the first sample splice site sequence (NIFᵥₐᵣ₋₁); wherein an NIFᵥₐᵣ₋₁ of 0 indicates that the sample splice site is abnormal;
   (c) wherein the risk of abnormal splicing of a sample splice site sequence from a subject is displayed by said computer interface.

In the method, step (b) may be repeated for one or more sample splice site sequence(s) comprised in the sample splice site from the subject, wherein each sample splice site sequence comprises non-identical set of nucleotides of the sample splice site, and wherein a NIFᵥₐᵣ of 0 (zero) for any sample splice site sequence indicates that the sample site is abnormal.

In a further embodiment, provided is a method of providing to an individual a risk of abnormal splicing of sample splice site from a subject, which is directly accessible by said individual through a computer interface, said method comprising

(a) providing a mechanism for said individual to input at least one sample splice site from a subject;
(b) determining a risk of abnormal splicing of the sample splice site sequence by
(i) obtaining a first sample splice site sequence comprised in the sample splice site from the subject;
(ii) determining a measure of Native Intron Frequency of the first sample splice site sequence (NIFᵥₐᵣ₋₁);
(iii) determining a Percentile (NIFᵥₐᵣ₋₁) of the first sample splice site sequence;
(iv) determining a measure of Native Intron Frequency of a first reference splice site sequence (NIF_{ref-1}); wherein the first reference splice site sequence and the first sample splice site sequence each originate from the same corresponding region of a gene;
(v) determining a Percentile (NIF_{ref-1}) of the first reference splice site sequence; and
(vi) determining the risk of abnormal splicing for the sample splice site by comparing the Percentile (NIFᵥₐᵣ₋₁) with the Percentile (NIF_{ref-1}) against a CSP reference database;
   (c) wherein the risk of abnormal splicing of the sample splice site is displayed by said computer interface.

In the method, step (b) may be repeated for one or more sample splice site sequence(s) comprised in the sample splice site from the subject, wherein each sample splice site sequence comprises non-identical set of nucleotides of the sample splice site, and wherein the risk of abnormal splicing for the sample splice site is determined by considering step (vi) for each sample splice site sequence together.

In a further embodiment, provided is a method of providing to an individual a risk of abnormal splicing of sample splice site from a subject, which is directly accessible by said individual through a computer interface, said method comprising

(a) providing a mechanism for said individual to input at least one sample splice site from a subject;
(b) determining a risk of abnormal splicing of the sample splice site sequence by
(i) obtaining a first sample splice site sequence comprised in the sample splice site from the subject;
(ii) determining a measure of Native Intron Frequency of the first sample splice site sequence (NIFᵥₐᵣ₋₁);
(iii) determining a measure of Native Intron Frequency of a first reference splice site sequence (NIF_{ref-1}); wherein the first reference splice site sequence and the first sample splice site sequence each originate from the same corresponding region of a gene; and
(iv) determining the risk of abnormal splicing for the sample splice site by comparing NIFᵥₐᵣ₋₁ with NIF_{ref-1} against a CSP reference database;
   (c) wherein the risk of abnormal splicing of the sample splice site is displayed by said computer interface.

In the method, step (b) may be repeated for one or more sample splice site sequence(s) comprised in the sample splice site from the subject, wherein each sample splice site sequence comprises non-identical set of nucleotides of the sample splice site, and wherein the risk of abnormal splicing for the sample splice site is determined by considering step (iv) for each sample splice site sequence together.

In a further embodiment, provided is a method of providing to an individual a risk of abnormal splicing of a sample splice site from a subject, which is directly accessible by said individual through a computer interface, said method comprising

(a) providing a mechanism for said individual to input at least one sample splice site from a subject;
(b) determining a risk of abnormal splicing of the sample splice site sequence by
(i) obtaining a first sample splice site sequence comprised in the sample splice site from the subject;
(ii) determining a clinical classification(s) associated with the nucleotide sequence of the first sample splice site sequence; and
(iii) determining the risk of abnormal splicing for the sample splice site by assessing the clinical classification(s) associated with the nucleotide sequence of the first sample splice site sequence determined in step (ii);
   (c) wherein the risk of abnormal splicing of the sample splice site is displayed by said computer interface.

In the method, step (b) may be repeated for one or more sample splice site sequence(s) comprised in the sample splice site from the subject, wherein each sample splice site sequence comprises non-identical set of nucleotides of the sample splice site, and wherein the risk of abnormal splicing for the sample splice site is determined by considering step (iii) for each sample splice site sequence together.

In a further embodiment, provided is a method of providing to an individual a risk of abnormal splicing of a sample splice site from a subject, which is directly accessible by said individual through a computer interface, said method comprising

(a) providing a mechanism for said individual to input at least one sample splice site from a subject;
(b) determining a risk of abnormal splicing of the sample splice site sequence by
(i) obtaining a first sample splice site sequence comprised in the sample splice site from the subject;
(ii) obtaining a first reference splice site sequence; wherein the first reference splice site sequence and the first sample splice site sequence each originate from the same corresponding region of a gene;
(iii) determining a clinical classification(s) associated with the nucleotide sequence of the first sample splice site sequence;
(iv) determining a clinical classification(s) associated with the nucleotide sequence of the first reference splice site sequence; and
(v) determining the risk of abnormal splicing for the sample splice site by assessing the clinical classification(s) associated with the nucleotide sequence of the first sample splice site sequence determined in step (iii) and the clinical classification(s) associated with the nucleotide sequence of the first reference splice site sequence determined in step (iv);
   (c) wherein the risk of abnormal splicing of the sample splice site is displayed by said computer interface.
   In the method, step (b) may be repeated for one or more sample splice site sequence(s) comprised in the sample splice site from the subject, wherein each sample splice site sequence comprises non-identical set of nucleotides of the sample splice site, and wherein the risk of abnormal splicing for the sample splice site is determined by considering step (v) for each sample splice site sequence together.

In one embodiment, provided is a method of providing to an individual a risk of abnormal splicing of a sample splice site from a subject, which is directly accessible by said individual through a computer interface, said method comprising

(a) providing a mechanism for said individual to input at least one sample splice site from a subject;
(b) determining a risk of abnormal splicing of the sample splice site sequence by
(i) obtaining a first sample splice site sequence comprised in the sample splice site from the subject;
(ii) determining a measure of Native Intron Frequency of the first sample splice site sequence (NIFᵥₐᵣ₋₁);
(iii) determining a Percentile (NIFᵥₐᵣ₋₁) of the first sample splice site sequence;
(iv) determining a measure of Native Intron Frequency of a first reference splice site sequence (NIF_{ref-1}); wherein the first reference splice site sequence and the first sample splice site sequence originate from the same corresponding region of a gene;
(v) determining a Percentile (NIF_{ref-1}) of the first reference splice site sequence;
(vi) calculating a lower bound and an upper bound for Percentile (NIFᵥₐᵣ₋₁) and calculating a lower bound and an upper bound for Percentile (NIF_{ref-1});
(vii) determining a range of NIF-shift by comparing the lower and upper bounds for Percentile (NIFᵥₐᵣ₋₁) with the lower and upper bounds for Percentile (NIF_{ref-1}) calculated in (vi);
(viii) identifying (a) similar NIF-shift variant(s), wherein a similar NIF-shift variant refers to a splice site sequence with a NIF-shift within the range of NIF-shift determined in (vii);
(ix) determining (a) clinical classification(s) associated with each similar NIF-shift variant identified in step (viii); and
(x) determining the risk of abnormal splicing for the sample splice site by assessing the clinical classification determined in step (ix) for each similar NIF-shift variant identified in step (viii).
   (c) wherein the risk of abnormal splicing of the sample splice site is displayed by said computer interface.
   In the method, step (b) may be repeated for one or more sample splice site sequence(s) comprised in the sample splice site from the subject, wherein each sample splice site sequence comprises non-identical set of nucleotides of the sample splice site; and wherein the risk of abnormal splicing for the sample splice site is determined by considering step (x) for each sample splice site sequence together.

In one embodiment, provided is a method of providing to an individual a risk of abnormal splicing of a sample splice site from a subject, which is directly accessible by said individual through a computer interface, said method comprising

(a) providing a mechanism for said individual to input at least one sample splice site from a subject;
(b) determining a risk of abnormal splicing of the sample splice site sequence by
(i) obtaining a first sample splice site sequence comprised in the sample splice site from the subject;
(ii) determining a measure of Native Intron Frequency of the first sample splice site sequence (NIFᵥₐᵣ₋₁);
(iii) determining a measure of Native Intron Frequency of a first reference splice site sequence (NIF_{ref-1}); wherein the first reference splice site sequence and the first sample splice site sequence originate from the same corresponding region of a gene;
(iv) calculating a lower bound and an upper bound for NIFᵥₐᵣ₋₁ and calculating a lower bound and an upper bound for NIF_{ref-1};
(v) determining a range of NIF-shift by comparing the lower and upper bounds for NIFᵥₐᵣ₋₁ with the lower and upper bounds for NIF_{ref-1}, calculated in (iv);
(vi) identifying (a) similar NIF-shift variant(s), wherein a similar NIF-shift variant refers to a splice site sequence with a NIF-shift within the range of NIF-shift determined in (v);
(vii) determining a clinical classification associated with each similar NIF-shift variant identified in step (vi); and
(viii) determining the risk of abnormal splicing for the sample splice site by assessing the clinical classification determined in step (vi) for each similar NIF-shift variant identified in step (vi).
   (c) wherein the risk of abnormal splicing of the sample splice site is displayed by said computer interface.
   In the method, step (b) may be repeated for one or more sample splice site sequence(s) comprised in the sample splice site from the subject, wherein each sample splice site sequence comprises non-identical set of nucleotides of the sample splice site; and wherein the risk of abnormal splicing for the sample splice site is determined by considering step (viii) for each sample splice site sequence together.

In a further embodiment, provided is a method of providing to an individual a risk of abnormal splicing of a sample splice, which is directly accessible by said individual through a computer interface, said method comprising

(a) providing a mechanism for said individual to input at least one sample splice site sequence from a subject;
(b) determining a risk of abnormal splicing of the sample splice site sequence by
(i) obtaining a first sample splice site sequence comprised in the sample splice site from the subject;
(ii) determining a measure of Native Intron Frequency of the first sample splice site sequence (NIFᵥₐᵣ₋₁);
(iii) determining a Percentile (NIFᵥₐᵣ₋₁) of the first sample splice site sequence;
(iv) determining a measure of Native Intron Frequency of a first reference splice site sequence (NIF_{ref-1}); wherein the first reference splice site sequence and the first sample splice site sequence each originate from the same corresponding region of a gene;
(v) determining a Percentile (NIF_{ref-1}) of the first reference splice site sequence;
(vi) determining a clinical classification(s) associated with the nucleotide sequence of the first sample splice site sequence;
(vii) optionally determining a clinical classification(s) associated with the nucleotide sequence of the first reference splice site sequence;
(viii) calculating a lower bound and an upper bound for Percentile (NIFᵥₐᵣ₋₁) and calculating a lower bound and an upper bound for Percentile (NIF_{ref-1});
(ix) determining a range of NIF-shift by comparing the lower and upper bounds for Percentile (NIFᵥₐᵣ₋₁) with the lower and upper bounds for Percentile (NIF_{ref-1}) calculated in (viii);
(x) identifying (a) similar NIF-shift variant(s), wherein a similar NIF-shift variant refers to a splice site sequence with a NIF-shift within the range of NIF-shift determined in (ix);
(xi) determining a clinical classification associated with each similar NIF-shift variant identified in step (x); and
(xii) determining the risk of abnormal splicing for the sample splice site by (1) comparing the Percentile (NIFᵥₐᵣ₋₁) with the Percentile (NIF_{ref-1}) against a CSP reference database, (2) assessing the clinical classification(s) associated with the nucleotide sequence of the first sample splice site sequence determined in step (v) and, optionally, the clinical classification(s) associated with the nucleotide sequence of the first reference splice site sequence (optionally) determined in step (vi); and (3) assessing the clinical classification determined in step (xi) for each similar NIF-shift variant identified in step (x);
   (c) wherein the pathogenic risk is displayed by said computer interface.
   In the method, step (b) may be repeated for one or more sample splice site sequence(s) comprised in the sample splice site from the subject, wherein each sample splice site sequence comprises non-identical set of nucleotides of the sample splice site; and wherein the risk of abnormal splicing for the sample splice site is determined by considering step (xii) for each sample splice site sequence together.

Mechanisms to input sequence data through a computer interface are well known in the art and include, but are not limited to, keyboard, disk drive, internet connection, etc.

Methods of treatment are also further embodiments of the methods herein described. Identification of a sample splice site associated with a gene known to be associated with an inherited disease (Mendelian disorder) or cancer provides a genetic diagnosis. The genetic diagnosis will direct applicable treatments for the particular disease or cancer. For example, cancer patients with a pathogenic splice site may be resistant to certain cancer treatment. In one embodiment provided is a method of treating a Mendelian disorder, said method comprising (a) determining a risk of abnormal splicing for a sample splice site; (b) diagnosing a Mendelian disorder or risk of a Mendelian disorder in view of the risk; and (c) administering a treatment for the diagnosed Mendelian disorder. In one embodiment, provided is a method of treating cancer, said method comprising (a) determining a risk of abnormal splicing for a sample splice site from a subject suffering from cancer; and (b) administering a cancer treatment that is amenable to cancers associated with an abnormal splice site. In one embodiment, provided is a method of treating a cancer in a subject suffering from cancer or at risk of suffering from cancer, said method comprising (a) determining a risk of abnormal splicing for a sample splice site from the subject; and (b) administering a splice-related cancer therapy. In one embodiment, provided is a method of treating and/or preventing cancer or a Mendelian disorder in a subject suffering from cancer or a Mendelian disorder or at risk of suffering from cancer or a Mendelian disorder comprising (a) determining a risk of abnormal splicing for a sample splice site from the subject; and (b) treating the subject by genetically editing the splice site determined to have an abnormal splice site.

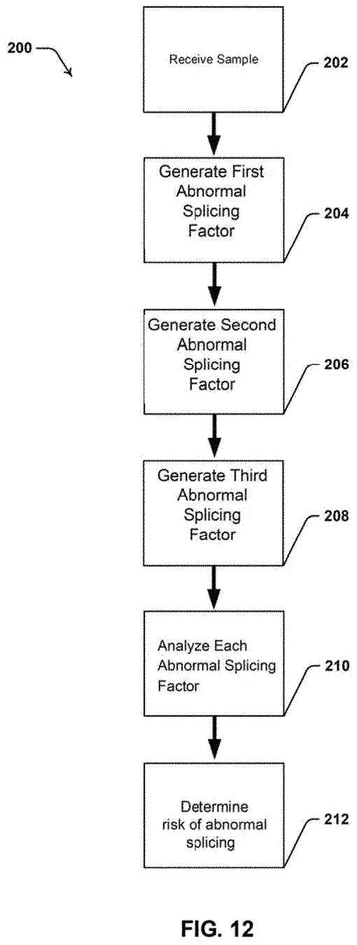

In a further embodiment, a method 200, illustrated schematically in FIG. 12 is presented for determining risk of abnormal splicing of a sample splice site. Method 200 begins when a sample splice site is received at step 202. A samples splice site sequence from the sample splice site is then compared to a corresponding reference splice site sequence to generate a first abnormal splicing factor at step 204. The first abnormal splicing factor is based on comparing a measure of Native Intron Frequency (NIF) of the sample splice site sequence (NIFᵥₐᵣ₋₁) and a NIF of a first reference splice site sequence (NIF_{ref-1}) against a CSP reference database and is described in greater detail below with reference to FIGS. 2B, 2C.

A second abnormal splicing factor is generated at step 206 by comparing a sample splice site sequence to pre-classified data. The pre-classified data includes variant splice sites which have been pre-classified as being either an abnormal splice site variant or benign variant splice site and is described in greater detail below with reference to FIG. 3B.

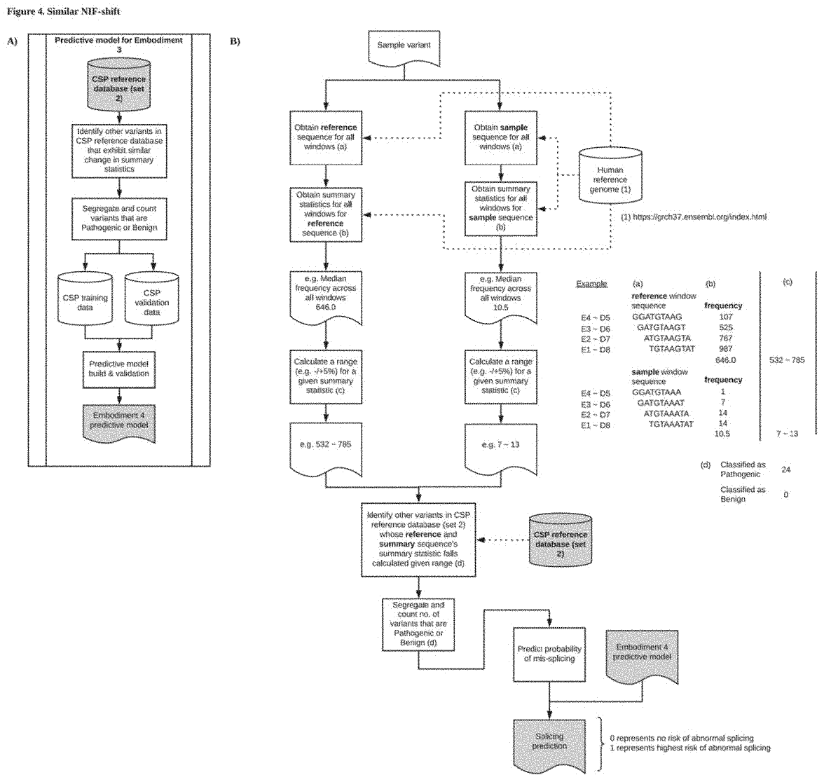

At step 208 a third abnormal splicing factor is determined based on similar NIFshift variant. The similar NIF-variants are based on pre-classified splice sites having a NIF-shift within a range of NIF-shift calculated from the NIF-shift of a sample splice site sequence and are described in detail with reference to FIG. 4B. The three abnormal splicing factors are then analysed at step 210 and a risk of abnormal splicing is determined at step 212.

It will be appreciated that there is no requirement to determine the abnormal splice site factors in the order described above and that reference to the terms “first”, “second” and “third” is not a reference to required order of determination. It will be appreciated that a method 200 may comprising determining the first and second abnormal splicing factors only or, alternatively, the first and third abnormal splicing factors only.

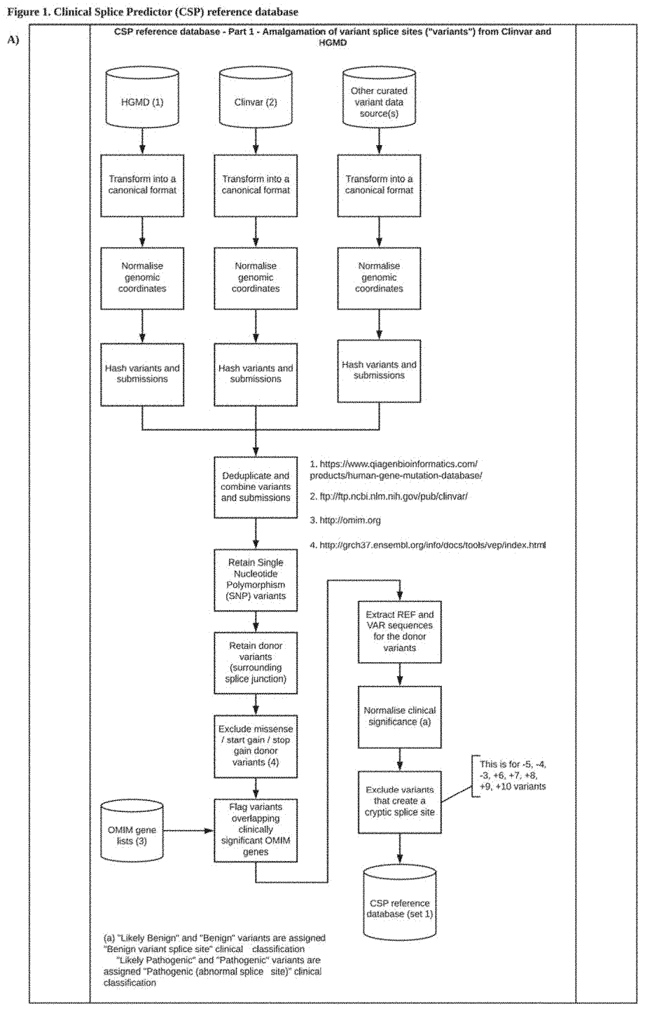

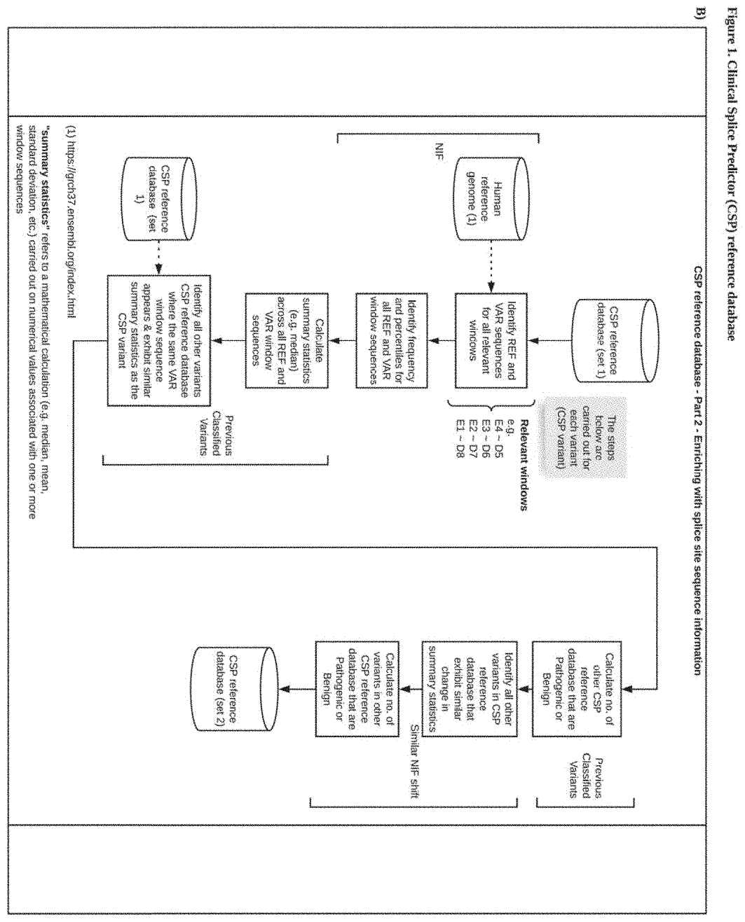

A risk of abnormal splicing for a sample splice site may be determined by comparing the abnormal risk factors to pre-classified data. In some embodiments, the pre-classified data is generated using method as exemplified in FIGS. 1A to 1C.

Pre-classified sample splice sites are taken from database comprising pre-classified data and compared to corresponding splice sites from a reference human genome sequence as exemplified in FIG. B.

Pre-classified abnormal splicing factors 204, 206 and 208 are then individually analysed 210 to produce a predictive algorithm as exemplified in FIGS. 2A and 3A. The analysis is a statistical analysis of factors 204, 206 and 208 to produce a model capable of taking abnormal splicing factors as an input and producing a risk of abnormal splicing as an output. In some embodiments, the algorithm is a logistic regression model generated by a machine learning algorithm

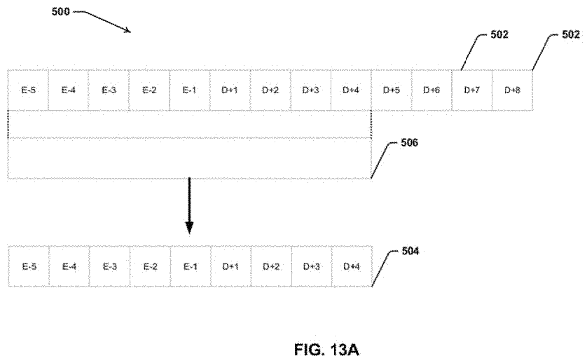

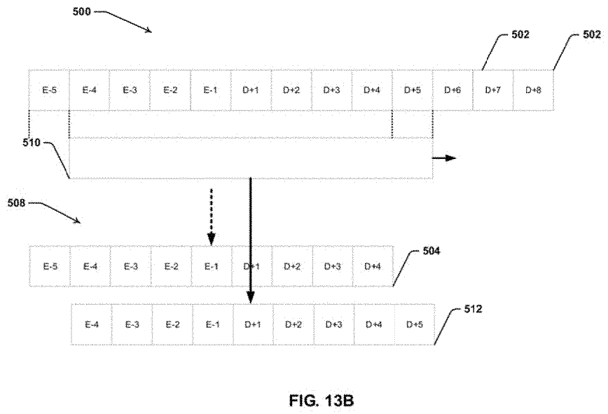

In some embodiments, exemplified in FIGS. 13A and 13B, one or more subsets of the nucleotides 500 of a sample splice sample 502 are used to generate abnormal splicing factors. A subset 504 is generated using a window 506 of predetermined length to select the nucleotides for subset 504 as shown in FIGS. 13A and 13B. In the illustrated example, window 502 is nine nucleotides in length and selects nucleotides at position E⁻⁵ to D⁺⁴ of a donor sample splice site. Each window 506 may be comprised of one or more regions of consecutive nucleotides. In certain embodiments, each window 506 may be comprised of one or more regions of consecutive nucleotides with one or more groups consisting of a single nucleotide.

In embodiments making use of a plurality of subsets 508, window 504 may be a sliding window 510, selecting a first subset 504 of nucleotides before sliding one nucleotide position along to generate the next subset 512 until the entire splice sample 500 is represented in subsets 508.

In a further embodiment, provided is a reference database comprising splice sites from a sequenced human genome. In certain embodiments, provide is a reference database comprising splice sites from a sequenced human genome, wherein each splice site sequence comprised in the reference data bases corresponds to a donor splice site. In certain embodiments, provide is a reference database comprising splice sites from a sequenced human genome, wherein each splice site sequence comprised in the reference data base comprises at least nucleotide positions E⁻⁵ to D⁺⁹ of a donor splice site or at least nucleotide positions E⁻⁵ to D⁺⁸ of a donor splice site.

In a further embodiment, provided is a Clinical Splice Predictor (CSP) reference database comprising variant splice sites with clinical classifications. In certain embodiments, provided is a CSP reference database comprising variant splice sites with clinical classifications, wherein each variant splice site comprised in the CSP reference database is classified as an abnormal splice site or as a benign variant splice site. In related embodiments, provided is a CSP reference database comprising variant splice sites with clinical classifications, wherein each variant splice site comprised in the CSP reference database is classified as an abnormal splice site or as a benign variant splice site and wherein a variant splice site classified as an abnormal splice site is also classified as a pathogenic splice site. In certain embodiments, provided is a CSP reference database comprising variant splice sites with clinical classifications, wherein each splice site sequence comprised in the CSP reference data bases corresponds to a donor splice site. In certain embodiments, provided is a CSP reference database comprising variant splice sites with clinical classifications, wherein each splice site sequence comprised in the CSP reference data base comprises at least nucleotide positions E⁻⁵ to D⁺⁹ of a donor splice site or at least nucleotide positions E⁻⁵ to D⁺⁸ of a donor splice site.

All references cited herein, including patents, patent applications, publications, and databases, are hereby incorporated by reference in their entireties, whether previously specifically incorporated or not.

### Example 1

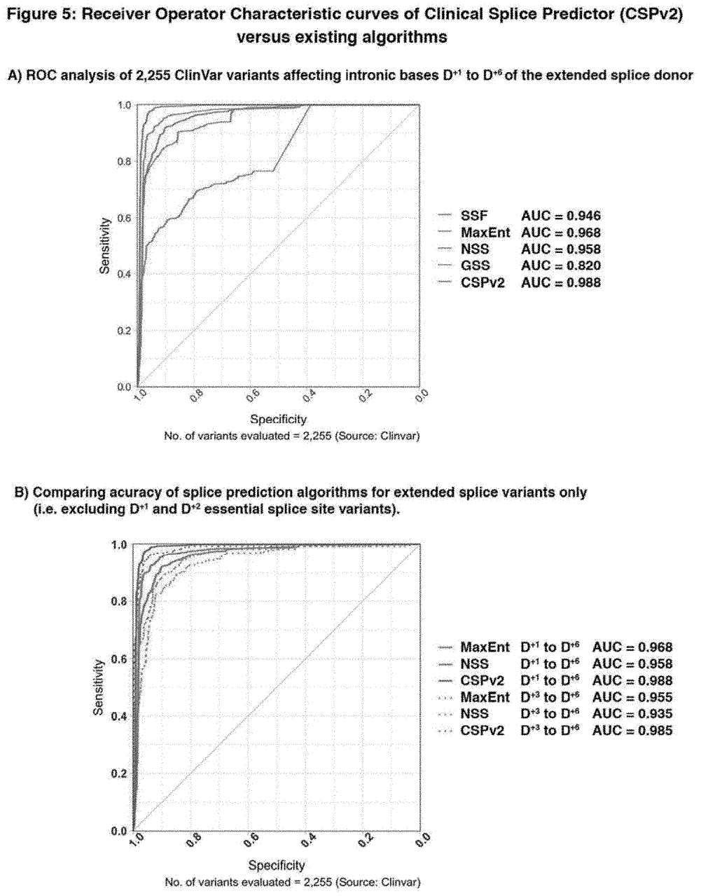

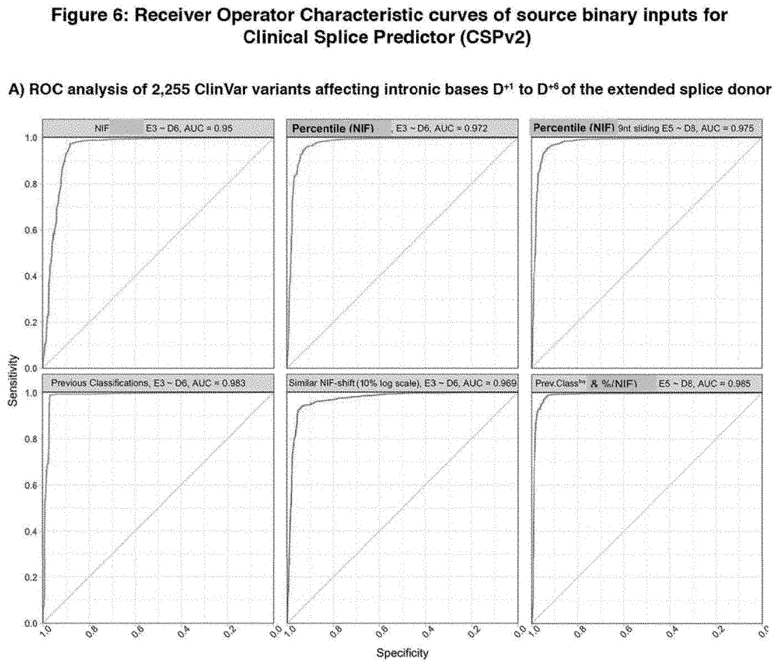

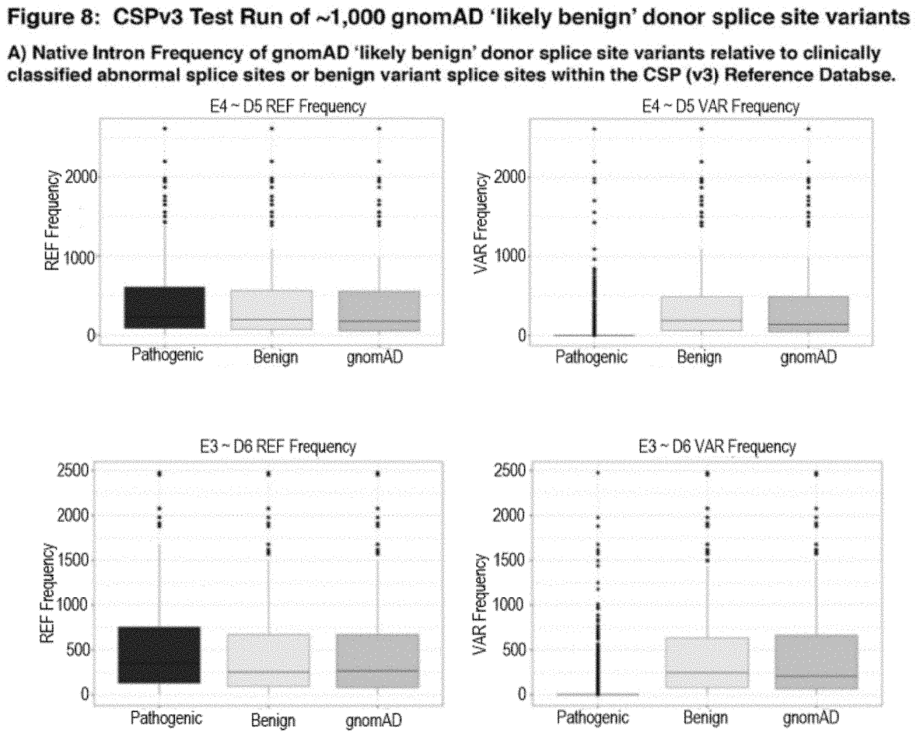

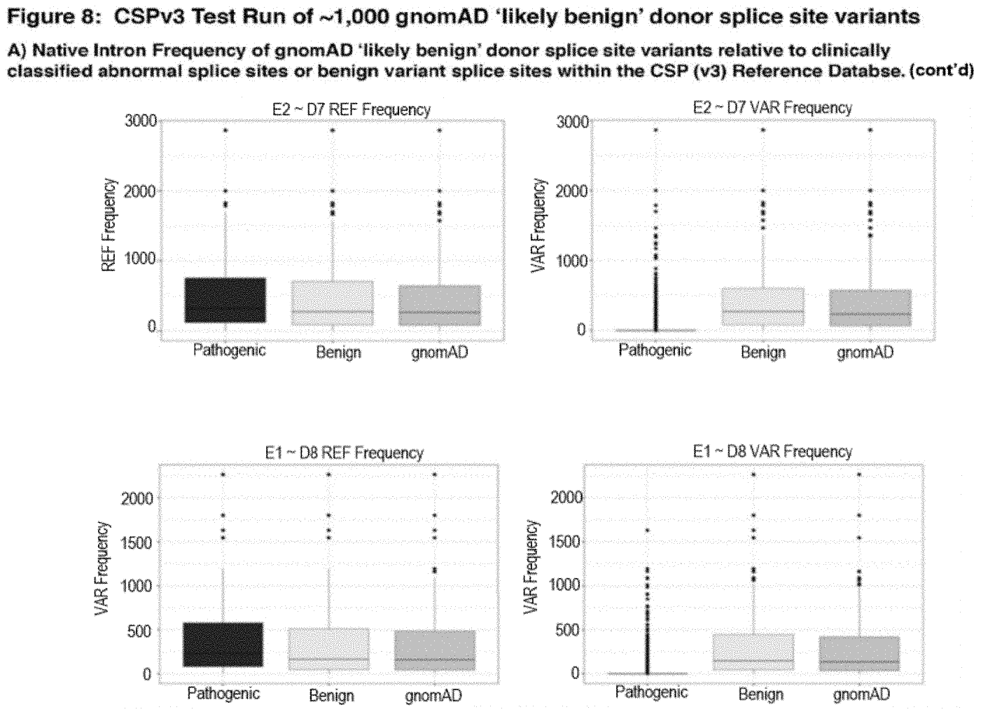

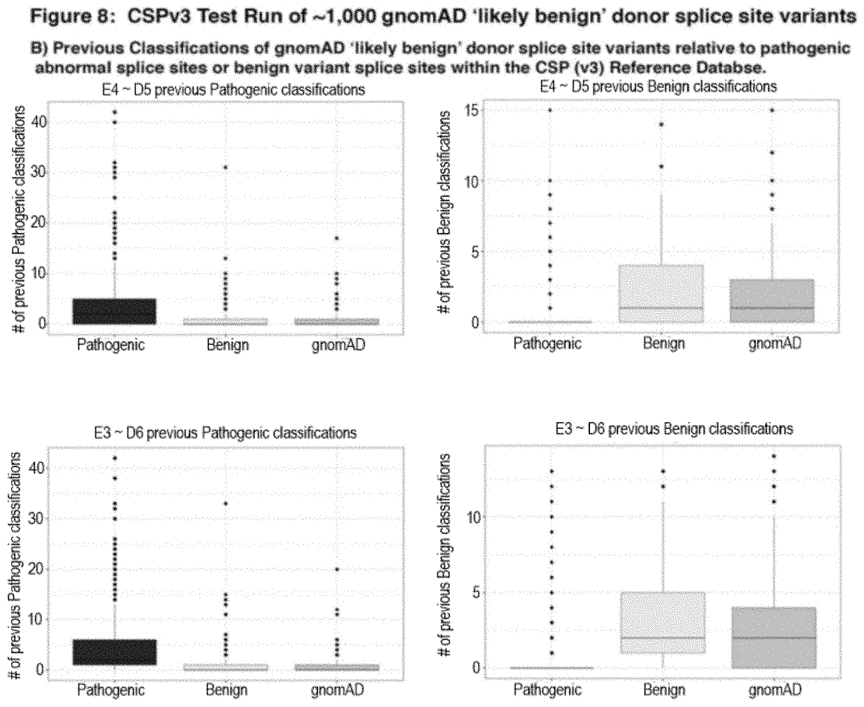

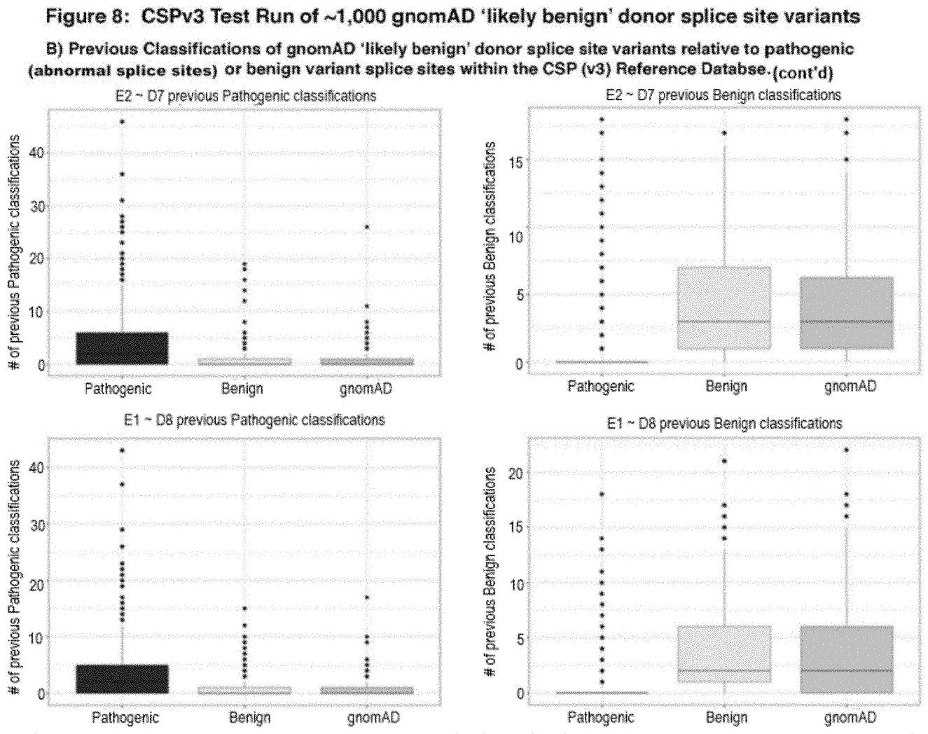

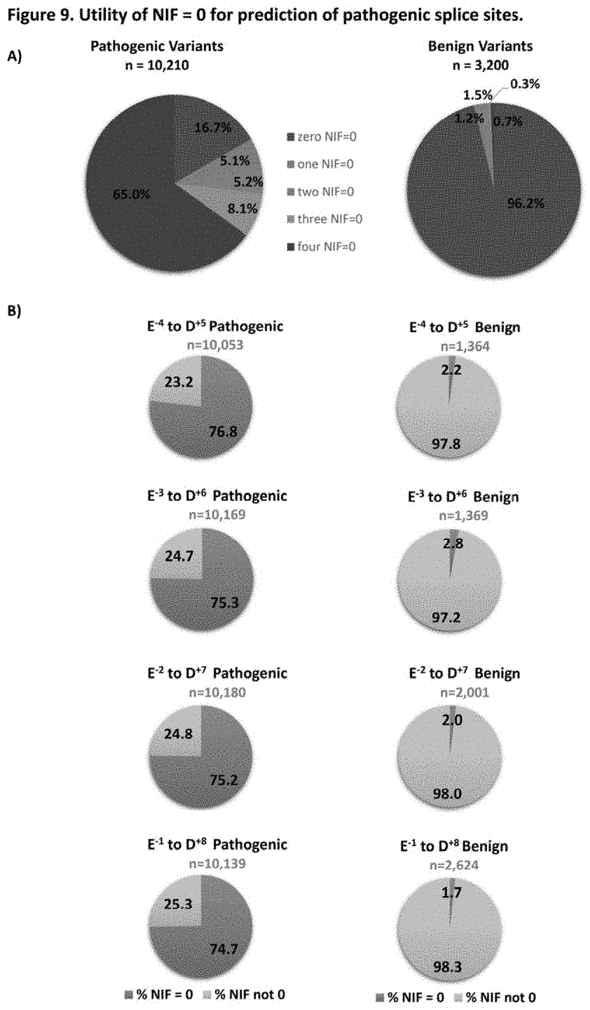

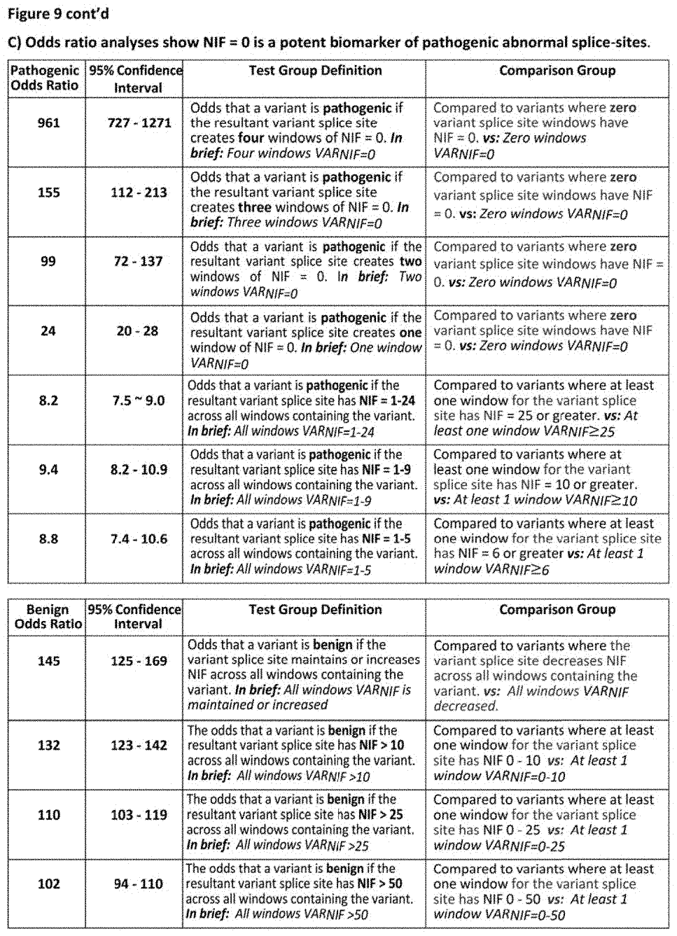

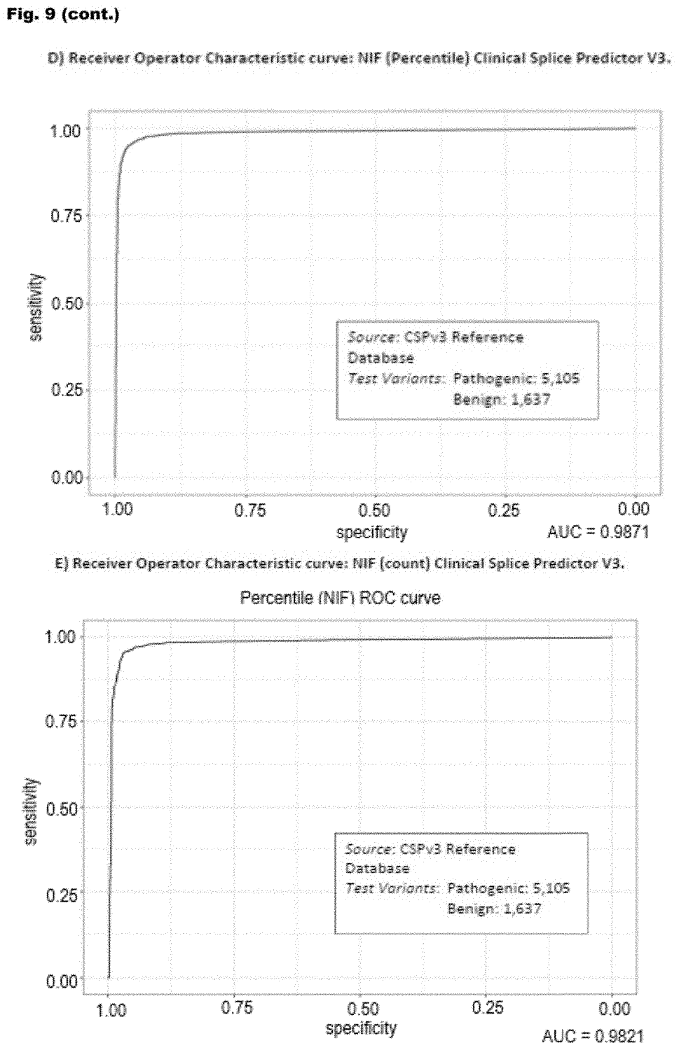

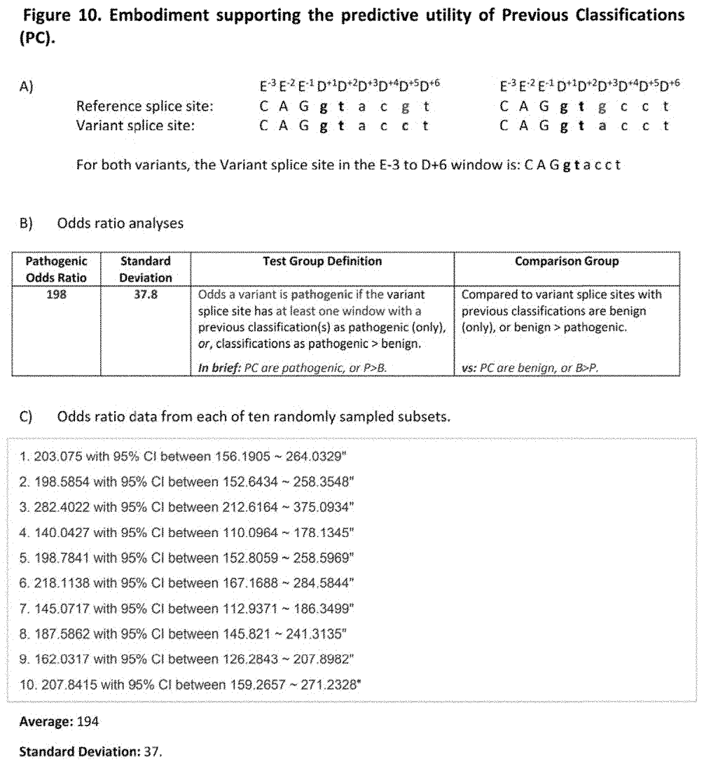

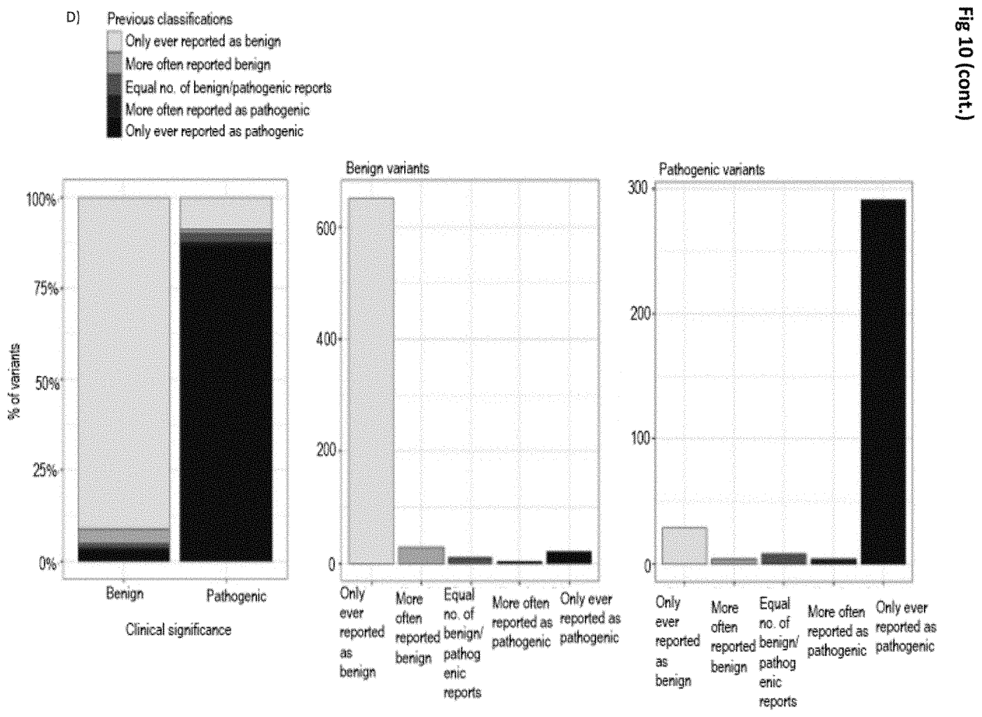

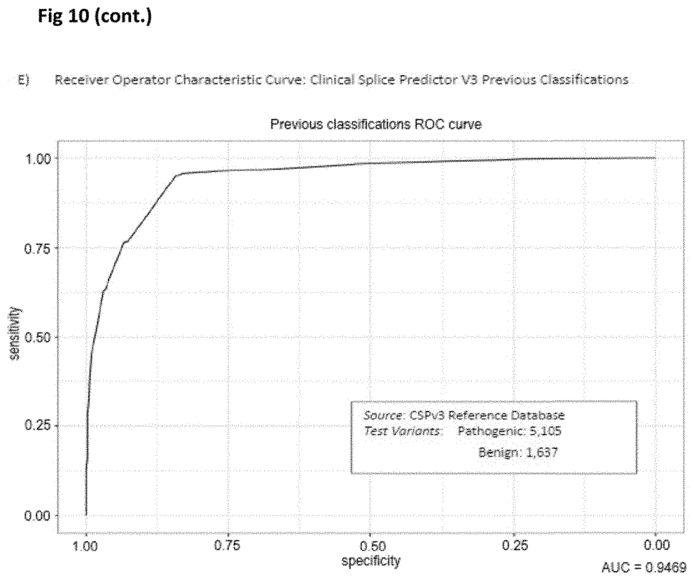

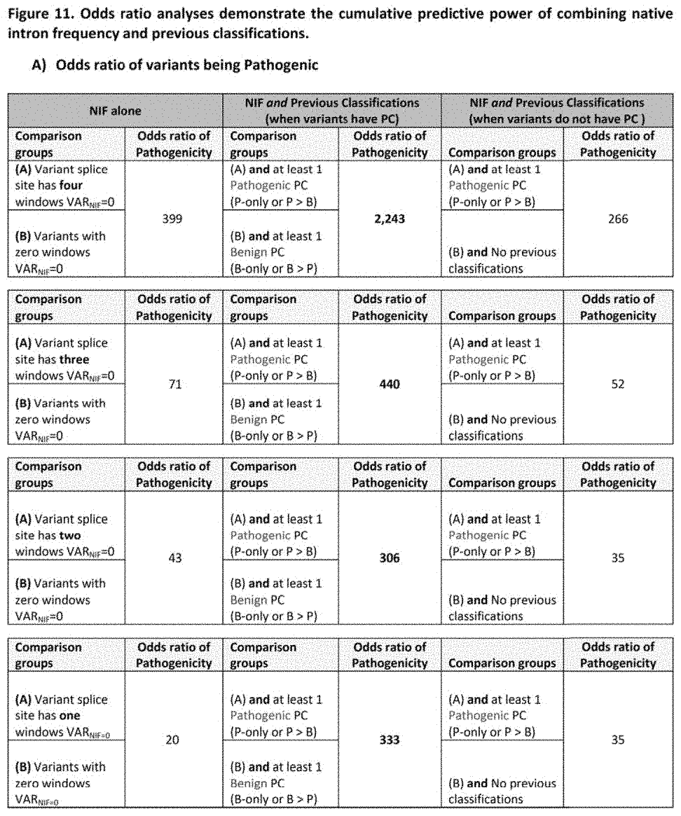

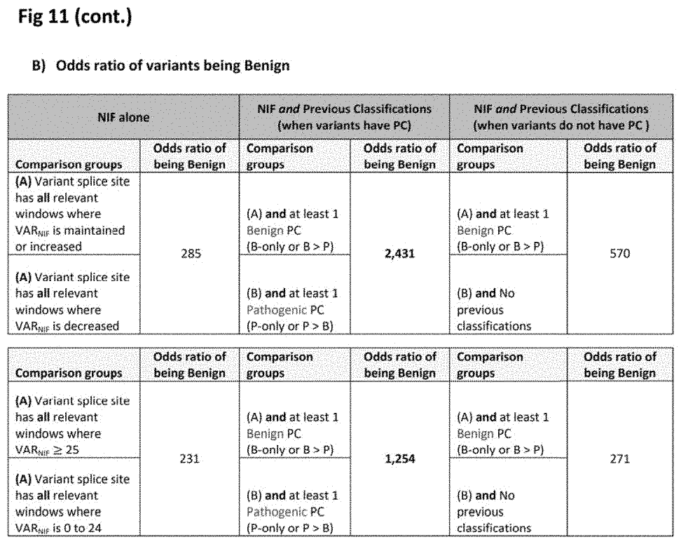

FIGS. 5 to 11 and 14 show generation of a Clinical Splice Predictor for identifying an abnormal splice site from a sample splice site by methods herein descried. For both CSP v2 and v3, the reference splice site sequences (reference human genome sequence) were derived from the “Genome Reference Consortium Build 37” (hg19), which was available from (<https://www.ncbi.nlm.nih.goviassembly/GCF 000001405.13>).

### Example 2

### Splicing Prediction Research Reports

Anonymised patient reports, which were generated subject to a confidentiality agreement. In each report, the risk of abnormal splicing of a sample splice site from a patient was assessed and the risk provided. The abnormal splicing of the splice site was confirmed by mRNA studies. In one report information under “Notes and Interpretation” was provided. In other reports, this information was not completed and while text is provided in the section, it is not associated with any information content.

### Example 3

Splicing Studies on mRNA

### Subject 1 (CLN5)

### Brief Clinical Summary Provided:

Neuronal Ceroid Lipofuscinosis (NCL)

### Results of Previous Genetic Testing:

Genetic testing of DNA extracted from blood of the affected individual identified a homozygous likely pathogenic variant in CLN5, c.320+5G>A

### CLN5 Chr13(GRCh37):g.77566411G>A

| Gene Name | Variant | Zygosity | Disease (MIM) | Inheritance | Parent Origin |
|---|---|---|---|---|---|
| CLN5 | c.320 + 5G > A | Homozygous | #256731 Ceroid | Autosomal | Both parents |
| (NM_006493.2) | | | Lipofuscinosis, | recessive | het |
| | | | Neuronal, 5 | | |

cDNA Studies Performed to Assess the Intronic Variant:

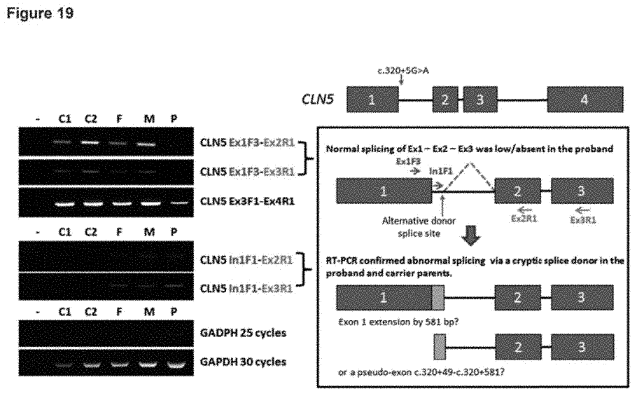

RT-PCR was performed on mRNA extracted from blood from the family trio (unaffected parents and affected individual). An abnormal pattern was observed for amplified cDNA products encompassing exons 1-2 and 1-3 of CLN5 in the proband (P) compared to controls (C1, C2) and the parental samples (F, M) (see FIG. 19).
A very low amount of CLN5 product was detected in the patient sample (P) in PCR reactions amplifying exons 1-2 or 1-3
A reduced amount of CLN5 product in PCR reactions amplifying exons 3-4.
Abnormal inclusion of intron-1 sequences into spliced products (see Figure, amplified cDNA products using intron-1 forward primer and exon 2 or exon 3 reverse primers). No product was detected in two controls (C1, C2); but all samples containing the c.320+5G>A variant (F, M, P) gave rise to a product encompassing part of intron 1 (ending at c.320+581) spliced to exon 2, indicating use of an alternative donor splice site. Amplification of GAPDH shows samples have similar amounts of total cDNA.

These data are suggestive of abnormal splicing of exon 1 in most CLN5 transcripts for the proband.

Possible consequences of the c.320+5G>A variant:

1) Omission of exon 1, with the mRNA beginning within exon 2
2) Abnormal Extension of exon 1 with inclusion of intron-1 sequences, and splicing from the cryptic intron-1 donor.
3) Omission of most/all of exon 1, with the mRNA beginning within the intron-1 pseudo-exon
4) Omission of part of exon 1, with inclusion of intronic sequences

No normally spliced exon 1-exon 2-exon-3 products were detected in the proband.

Inclusion of intronic sequences will induce a damaging effect for the encoded CLN5 protein.

### Conclusions:

mRNA studies confirm the homozygous CLN5 c.320+5G>A variant induces abnormal splicing of CLN5 transcripts.

All detected abnormal splicing events are likely to render the encoded CLN5 protein dysfunctional/non-functional.
No normal spliced exon 1-exon 2-exon 3 products were detected in the proband.

Collective data are consistent with likely pathogenicity of the CLN5c.320+5G>A variant.

Homozygous variants in CLN5 are consistent with the phenotype of neuronal ceroid lipofuscinosis in the affected individual.

### Subject 2 (CC2D2A)

### Brief Clinical Summary Provided:

Congenital hypotonia.

### Results of Previous Genetic Testing:

Homozygous class 4 variant in RYR1

### Chr19:g.38980890G>A; NM 000540.2:c.5989G>A; p. (Glu1997Lys).

Homozygous variant of uncertain significance in CC2D2A:

### Chr4:g.15504547G>T

NM_001080522.2:c.438+1G>T

This variant has not previously been reported in ClinVar. This variant is not present in the Genome Aggregation Database (gnomAD).

### CC2D2A Chr4(GRCh37):a.15504547G>T

| | | | Disease | | |
|---|---|---|---|---|---|
| Gene Name | Variant | Zygosity | (MIM) | Inheritance | Parent Origin |
| CC2D2A | c.438 + 1G > T | Homozygous | #612285 | Autosomal | Both parents are |
| (NM_001080522.2) | | | Joubert | recessive | heterozygous |
| | | | syndrome 9 | | carriers |

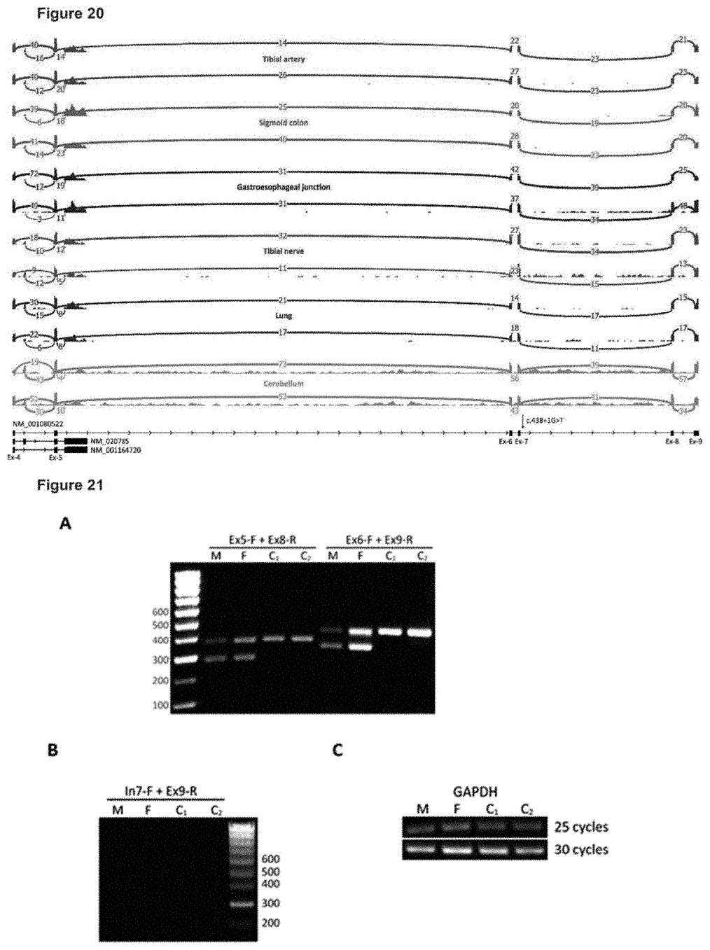

FIG. 20 Sashimi plots showing RNA sequencing (RNAseq) coverage across CC2D2A exons 4-9 (NM 001080522) derived from tibial artery, sigmoid colon, gastroesophageal junction, tibial nerve, lung and cerebellum. There are two short isoforms and one long isoform of CC2D2A. The c.438+1G>T variant is downstream of the 3′UTR of the short isoforms and therefore only predicted to affect the long CC2D2A isoform. The long isoform is the predominant transcript, although this varies (≈50-95% of CC2D2A transcripts) depending on the tissue from which the RNA is derived. Exon-7 is a canonical exon of the long CC2D2A isoform. RNAseq data obtained from the Genotype-Tissue Expression (GTEx) Project.

Conclusions
mRNA studies confirm CC2D2A c.438+1G>T variant induces abnormal splicing of CC2D2A transcripts in blood RNA.
Detection of one abnormal splicing event, in-frame exon-7 skipping. This event removes 34 amino acids p. (Ser113 Glu146del) from the CC2D2A protein, of which 24 residues are conserved in mammals.
Exon-7 is canonical in the predominant CC2D2A isoform (long isoform) across multiple tissues. The c.438+1G>T variant is not predicted to affect the two short isoforms of CC2D2A.
   mRNA Studies Performed to Assess the c.438+1G>T Variant:
   Summary of Results in mRNA Derived from Blood

RT-PCR was performed on mRNA extracted from the whole blood taken from the unaffected parent carriers of the c.438+1G>T variant.

We detected one abnormal splicing event resulting from the c.438+1G>T variant:
1. Exon-7 skipping (FIG. 21A, Band #2)

We also detected normal splicing of CC2D2A transcripts in all samples (FIG. 21A, Band #1).

RT-PCR of CC2D2A mRNA Isolated from Blood (FIG. 21).

A) Using two sets of primers flanking the c.438+1G>T variant we detect one abnormally sized band in the maternal and paternal samples (Band #2). Sanger sequencing confirmed this band corresponds to exon-7 skipping. We also detect normal exon-6-7-8 splicing in all samples (Band #1), consistent with both parents being heterozygous carriers of the c.438+1G>T variant.

B) Using a forward primer in intron-7 and a reverse primer in exon-9 we were unable to detect intron retention or use of a cryptic 5′-splice site.

C) Amplification of GAPDH demonstrates cDNA loading. Replicate samples were subject to PCR for 25 or 30 cycles in order to confirm the PCR cycling conditions were sub-saturating and able to detect lower levels or quality of a specimen. Lanes: Mother (M), Father (F), Control 1 (C1) (female, 24 years), Control 2 (C2) (male, 31 years).

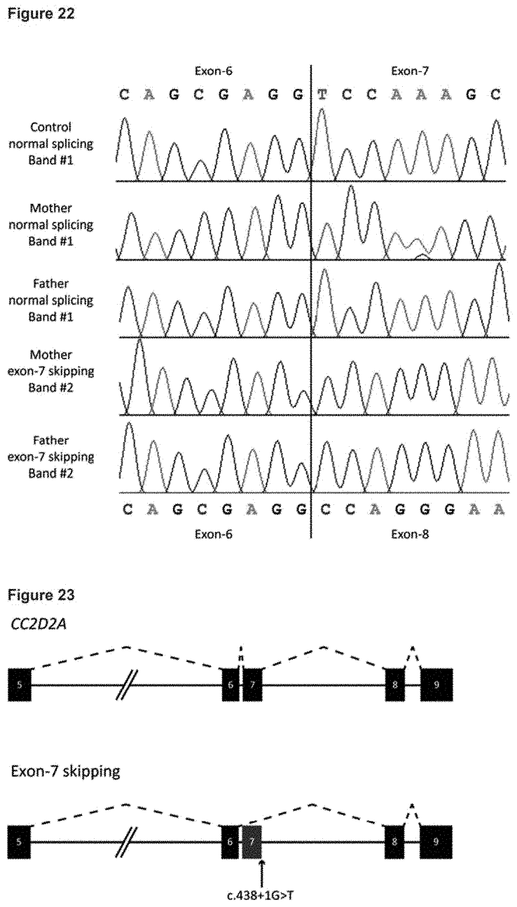

Sanger sequencing of RT-PCR amplicons showed the abnormally sized Band #2 in the maternal and paternal samples was due to exon-7 skipping (FIG. 22).

Schematic of the splicing abnormality induced by the c.438+1G>T variant. (FIG. 23)

### Consequences for the Encoded CC2D2A Protein:

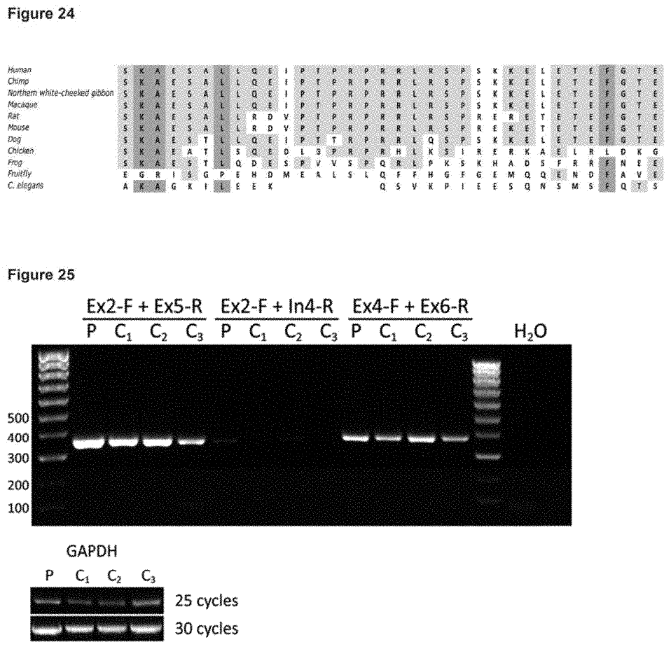

The c.438+1G>T variant results in exon-7 skipping, an in-frame event. Exon-7 skipping removes 34 amino acids p. (Ser113 Glu146del) from the CC2D2A protein, of which 24 residues are conserved in mammals as shown in FIG. 24.

### Subject 3 (CACNA1E)

### Brief Clinical Summary Provided:

Intellectual disability, epilepsy and cardiac arrhythmia.

### Results of Previous Genetic Testing:

Exome sequencing identified a heterozygous variant in CACNA1E gene:

Chr1(GRCh37):g.181547008G>A

### NM_001205293.1(CACNA1E):c.616+3G>A

p.?

This variant has not previously been reported in ClinVar. This variant is not present in the Genome Aggregation Database (gnomAD).

### CACNA1E Chr1(GRCh37):g.181547008G>A

| | | | | | Parent |
|---|---|---|---|---|---|
| Gene Name | Variant | Zygosity | Disease (MIM) | Inheritance | Origin |
| CACNA1E | c.616 + 3G > A | Heterozygous | Not OMIM listed. | Autosomal | Presumed |
| (NM_001205293.1) | | | | dominant | de novo |

### Conclusions

No evidence for abnormal splicing induced by the CACNA1E c.616+3G>A variant was found.

CACNA1E exon-4 is a canonical exon included in all RefSeq CACNA1E isoforms. Therefore splicing outcomes observed in blood RNA hold relevance to the predominant CACNA1E isoform expressed in brain.

mRNA Studies Performed to Assess the Extended Splice Site Variant:

RT-PCR was performed on mRNA extracted from the whole blood of the affected individual. We found no evidence for abnormal splicing FIG. 25. Specifically, RT-PCR of PIGN mRNA isolated from blood. FIG. 25 A No abnormal splicing was detected using 3 primer combinations. Intron 4 retention was detected in the patient and three controls (red arrows). FIG. 25 B GAPDH demonstrates similar cDNA loading. Lanes: Patient (P), control 1 (C1) (female, 26 years), control 2 (C2) (female, 27 years), control 3 (C3) (male, 3 weeks).

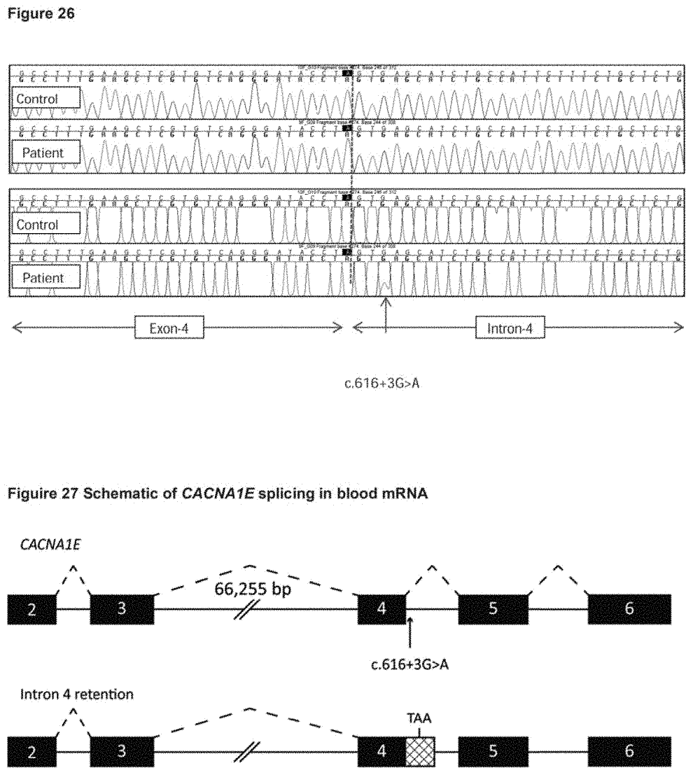

Sanger sequencing of RT-PCR amplicons confirmed intron-4 retention in the patient and controls. Levels of intron-4 retention from the c.616+3G>A variant containing allele may be reduced due to the predicted strengthening of the exon-4 5′ splice site. No common SNPs were amplified by our RT-PCRs to investigate allele imbalance. FIG. 26

### Subject 4 (ASNS)

### Brief Clinical Summary Provided:

Microcephaly and pontocerebellar hypoplasia.

### Results of Previous Genetic Testing:

Previous genetic testing identified a homozygous essential splice site variant in ASNS:

Chr7(GRCh37):g.97482371C>T

### NM_001673.4(ASNS):c.1476+1G>A

p.?

### ASNS Chr7(GRCh37):g.97482371C>T

| | | | | | Parent |
|---|---|---|---|---|---|
| Gene Name | Variant | Zygosity | Disease (MIM) | Inheritance | Origin |
| ASNS | c.1476 + 1G > A | Homozygous | #615574 | Autosomal | Mother and |
| (NM_001673.4) | | | Asparagine | recessive | father are |
| | | | Synthetase | | heterozygous |
| | | | Deficiency; | | carriers |
| | | | ASNSD | | |

Conclusions
1. Our RT-PCR results confirm the c.1476+1G>A variant induces abnormal splicing; with no evidence for residual normal splicing (though levels may be below that detected by our approaches). All abnormal splicing events exert a damaging effect for the encoded asparagine synthetase protein.
   a. Exon-12 skipping induced by the ASNS c.1476+1G>A variant abnormally removes 52 amino acids from the encoded asparagine synthetase protein.
   b. Use of the Exon-12 cryptic 5′splice-site abnormally removes 16 amino acids from the encoded asparagine synthetase protein.
   c. Retention of introns-11, intron-12 or both intron-11 and 12 each result in introduction of a premature termination codon.
2. ASNS exon-12 is a canonical exon included in all predominant ASNS isoforms expressed in brain. Therefore splicing outcomes observed in blood and fibroblast RNA hold inference to the predominant ASNS isoform in brain.
3. Studies of mRNA derived from fibroblasts obtained from the deceased sibling showed an identical pattern of abnormal splicing induced by the c.1476+1G>A variant; exon-12 skipping, use of an exon-12 cryptic 5′-splice site, retention of intron-11 and/or intron-12.

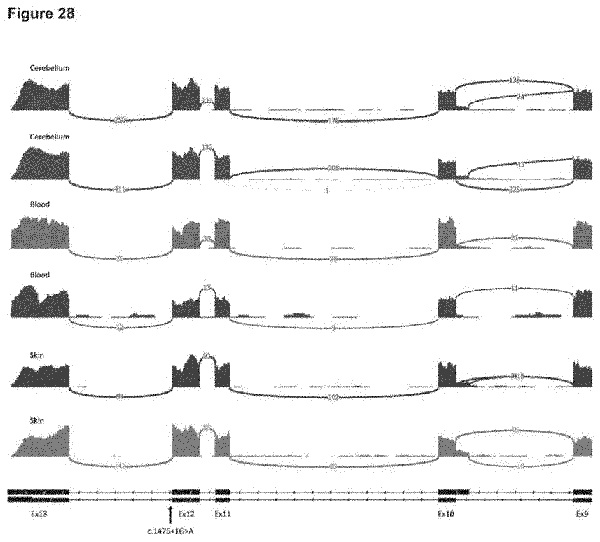

FIG. 28. Sashimi plots showing RNA sequencing coverage across ASNS exons 9-13 in RNA derived from two brain samples (red, female, 19 weeks; blue, female, 37 weeks); two blood samples (green, male, 49 years; brown, female, 30 years; purple, female, 11 years); and two skin samples (purple, male, 57 years; orange, male, 61 years). ASNS exon-12 is a canonical exon included in all predominant ASNS isoforms expressed in brain, blood and skin.

mRNA Studies to Assess the ASNS Essential Splice-Site Variant and Consequences for the Encoded Asparagine Synthetase Protein
Summary of results in blood mRNA

RT-PCR was performed on mRNA extracted from the whole blood of the proband and his unaffected parents.

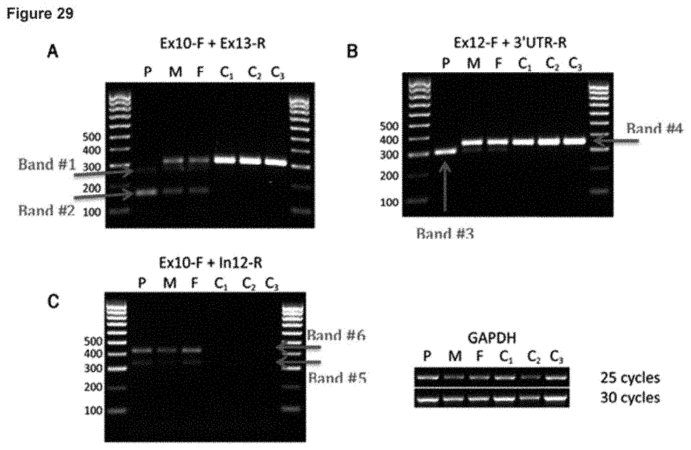

RNA studies of ASNS cDNA derived from whole blood gave robust PCR results. We found no evidence of normal splicing in the patient sample using six different primer combinations. We detect four predominant abnormal splicing events (FIG. 29):
1. Exon-12 skipping abnormally removes 156 nucleotides from the ASNS pre-mRNA. This event is in frame, deleting 52 amino acids p. (Asn441_Gln492del) from the encoded protein (FIG. 3, Band #2).
2. Use of a cryptic 5′ splice-site removes 48 nucleotides upstream of the native exon 12. This event is in-frame, deleting 16 amino acids p. (Lys478_Val493del) from the encoded protein (FIG. 29, Band #1).
3. Intron retention:
   a. Ectopic inclusion of 89 nucleotides of intron 11 including a premature termination codon (FIG. 29, Band #6).

Ectopic inclusion of at least 57 nucleotides of intron 12 including a premature termination codon (FIG. 29, Band #5).

FIG. 29 RT-PCR of ASNS mRNA isolated from blood. A) Using primers flanking the c.1476+1G>A variant (exon-10 forward and exon-13 reverse) we detected two abnormally sized bands in the patient and parental samples, relative to three controls. Sanger sequencing (FIG. 4) confirmed Band #1 corresponds to use of a cryptic 5′ splice-site, 48 nucleotides upstream of the native 5′ splice-site; and Band #2 corresponds to exon 12 skipping. B) Using a forward primer in exon 12 and a reverse primer in the 3′UTR of ASNS, the proband shows exclusive use of the cryptic 5′ splice-site in exon 12 (Band #3). We find no evidence for normal exon 12 to exon 13 splicing in the affected neonate. Parental samples showed both; 1) normal exon 12 to exon 13 splicing (Band #4) and 2) use of the exon 12 cryptic 5′ splice-site (Band #3), consistent with heterozygosity of the c.1476+1G>A variant. C) Use of a reverse primer in intron 12 shows abnormal inclusion of intronic sequence in the patient, and parental samples, that was not detected in controls. Band #5 corresponds to intron 12 inclusion and Band #6 corresponds to the inclusion of intron 11 and intron 12. D) Amplification of GAPDH demonstrates similar cDNA loading. Lanes: Patient (P), mother (M), father (F), control 1 (C1) (male, 7 months), control 2 (C2) (male, 5 years), control 3 (C3) (Female, 43 years).

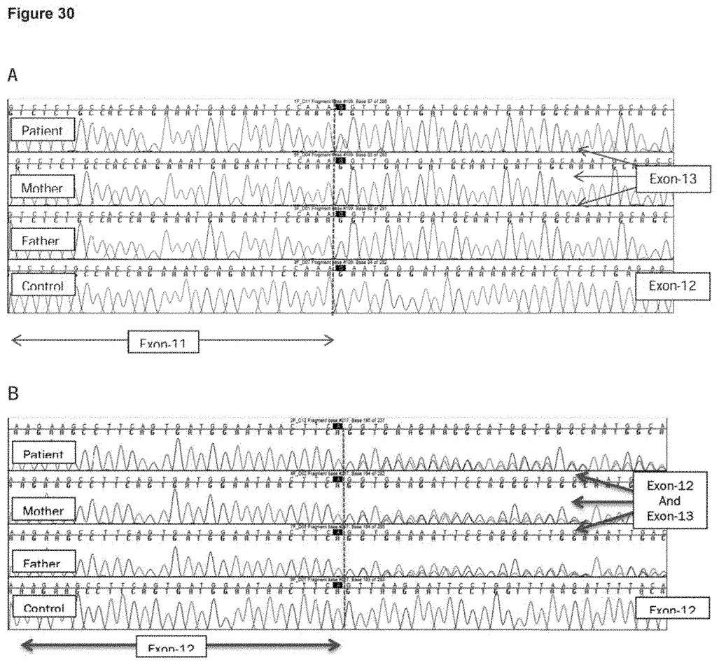

FIG. 30 Sanger sequencing of RT-PCR amplicons. A) Chromatogram showing the abnormal sized Band #2 in the patient and parental samples were due to exon-12 skipping. B) Chromatogram showing the abnormal sized Band #1 and #3 in the patient and parental samples were due to the use of the cryptic 5′ splice-site within exon 12. ASNS transcripts with normal splicing from exon 12 to exon 13 were detected in the parental samples, but not detected in the proband.

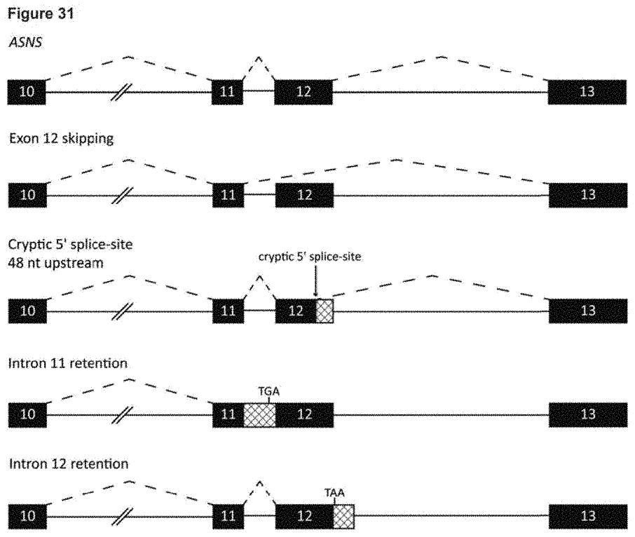

### FIG. 31: Schematic of the Splicing Abnormalities Induced by the c.1476+1G>A Variant.

### Consequences for the Encoded ASNS Protein:

Exon-12 skipping abnormally removes 156 nucleotides from the ASNS mRNA, deleting 52 amino acids p. (Asn441_Gln492del) from the encoded asparagine synthetase protein.

Use of the Exon 12 cryptic 5′splice-site abnormally removes 48 nucleotides from exon 12, deleting 16 amino acids p. (Lys478_Val493del) from the encoded asparagine synthetase protein.

Retention of intron 11, or intron 12, or both intron 11 and 12—results inclusion of intronic sequence into the ASNS mRNA transcript. In all cases (retention of intron 11, intron 12 or both intron 11 and 12) the resultant abnormal mRNA encodes a premature termination codon, and thus may be targeted by nonsense-mediated decay. Any ASNS transcripts escaping nonsense-mediated decay encode asparagine synthetase proteins lacking a complete asparagine synthetase enzymatic domain, and are therefore likely to be dysfunctional/non-functional.

All splicing outcomes impact the asparagine synthetase domain (p. 213-536) and are consistent with a damaging effect on the asparagine synthetase protein.

### Subject 5 ARMC4:

### Brief Clinical Summary Provided:

Primary ciliary dyskinesia.

### Results of Previous Genetic Testing:

Previous genetic testing identified two compound heterozygous variants in ARMC4:

Variant of Uncertain Significance

### Chr10(GRCh37):g.28233146C>G

### NM_018076.4(ARMC4):c.1743+5G>C

p.?

This variant has not previously been reported in ClinVar. This variant is not present in the Genome Aggregation Database (gnomAD).

Nonsense Variant

### Chr10(GRCh37):g.28149735G>T

### NM_018076.4(ARMC4):c.2840C>A

### p. (Ser947*)

This variant has previously been reported in ClinVar. This variant is present in the Genome Aggregation Database (gnomAD) at an allele frequency of 0.000007969 (1/125486).

### ARMC4 Chr10(GRCh37):g.28233146C>G

### ARMC4 Chr10(GRCh37):g.28149735G>T

| | | | | | Parent |
|---|---|---|---|---|---|
| Gene Name | Variant | Zygosity | Disease (MIM) | Inheritance | Origin |
| ARMC4 | c.1743 + 5G > C | Heterozygous | #615451 | Autosomal | Paternal |
| (NM_018076.4) | | | Ciliary dyskinesia, | recessive | |
| | | | primary, 23; CILD23 | | |
| | c.2840C > A | Heterozygous | | | Maternal |

Conclusions
1. mRNA studies indicate the heterozygous ARMC4 c.1743+5G>C variant induces abnormal splicing of ARMC4 transcripts in mRNA from a skin biopsy taken from the heterozygous parent carrier (father) of the variant.
2. We detect increased levels of ARMC4 exon-12 skipping relative to normal splicing of exons 11-12-13 in the parental carrier of the c.1743+5G>C variant, relative to controls. Exon-12 skipping is in-frame, removing 70 amino acids p. (Ile512_Leu581del) from the conserved Armadillo domain of ARMC4.
3. Collective results indicate the allele bearing the ARMC4 c.1743+5G>C variant predominantly produces ARMC4 transcripts with exon-12 skipping. However, interpretation of results remains challenging, as natural exon-12 skipping is observed in controls, across multiple tissues. We are unable to definitively determine whether the paternal allele bearing c.1743+5G>C variant manifests complete or partial mis-splicing.
4. Among the 70 residues removed by ARMC4 exon-12 skipping, 30 residues are conserved from mammals to fruit-fly, and a further 18 residues are conserved from mammals to zebrafish. Conservation of 48/70 deleted residues throughout vertebrate evolution strongly support their functional importance.
5. Exon-12 is included in all predominant ARMC4 isoforms across multiple tissues.
6. If ARMC4 is phenotypically concordant with the affected individual's presentation, we consider recessive inheritance of the c.1743+5G>C splicing variant in trans with the c.2840C>A nonsense variant molecularly consistent as plausible causal variants, due to deficiency of encoded full-length ARMC4 protein.

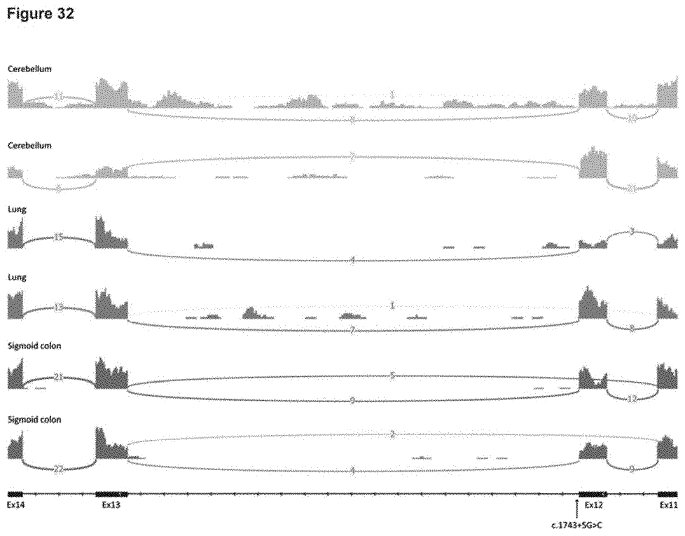

FIG. 32 Sashimi plots showing RNA sequencing (RNAseq) coverage across ARMC4 exons 11-14 in RNA derived from cerebellum, lung and sigmoid colon. ARMC4 exon-12 is included in the predominant isoform and exon-12 skipping is a normal low frequency event. RNAseq data obtained from the Genotype-Tissue Expression (GTEx) Project.

mRNA Studies Performed to Assess the c.1743+5G>C Variant:
Summary of Results in mRNA Derived from Skin

RT-PCR was performed on mRNA extracted from the skin of the unaffected father.

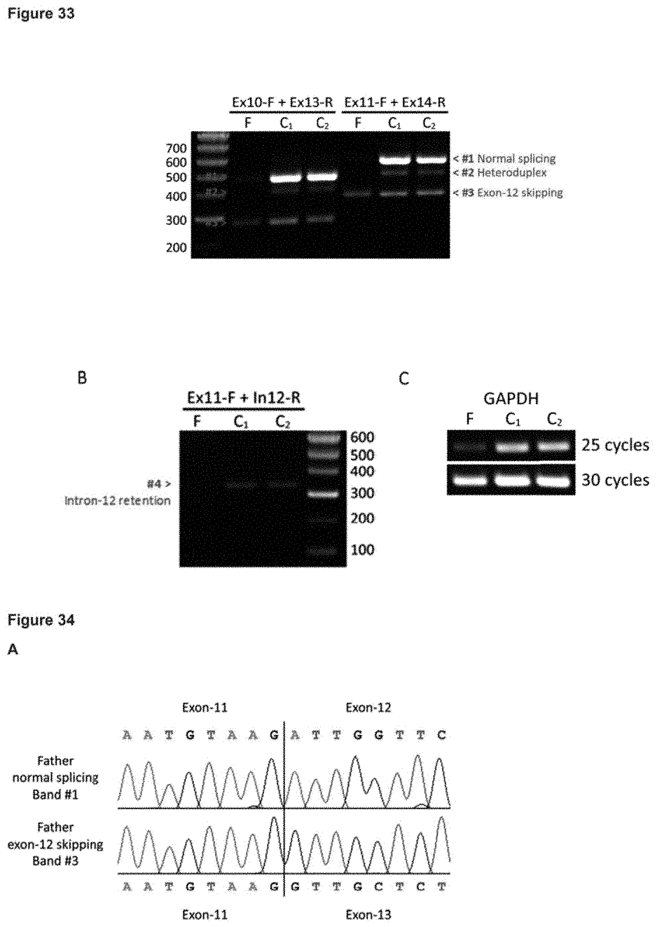

In the paternal and control samples we detect:
1. Normal exon-11-12-13 splicing (FIG. 33A, Band #1)
2. Exon-12 skipping (FIG. 33A, Band #3)

In control samples we also detect:
1. A heteroduplex amplicon of both normal splicing and exon-12 skipping (FIG. 33A, Band #2)

Intron-12 retention (FIG. 33B, Band #4)

FIG. 33

RT-PCR of ARMC4 mRNA isolated from skin.
A) Using two sets of primers flanking the c.1743+5G>C variant we detect three amplicons:
Band #1: Normal exon-11-12-13 splicing (paternal and control samples).
Band #2: Heteroduplex (controls only).
Band #3: Exon-12 skipping (paternal and control samples).
B) Using a reverse primer in intron-12 we detect intron-12 retention in control samples (Band #4)*. Intron-12 retention was not detected in the paternal sample.
C) Amplification of GAPDH demonstrates cDNA loading. Replicate samples were subject to PCR for 25 or 30 cycles in order to confirm the PCR cycling conditions were sub-saturating and able to detect lower levels or quality of a specimen. Lanes: Father (F), Control 1 (C₁) (male, 48 years), Control 2 (C₂) (male, 52 years)
FIG. 34, Sanger sequencing of RT-PCR amplicons.
A) In the paternal sample:
Band #1 corresponds to normal splicing
Band #3 corresponds to exon-12 skipping
B) and C) In control samples:
Band #1 corresponds to normal splicing
Band #2 is a heteroduplex of DNA consisting of normal splicing and exon-12 skipping
Band #3 corresponds to exon-12 skipping
Band #4 corresponds to intron-12 retention

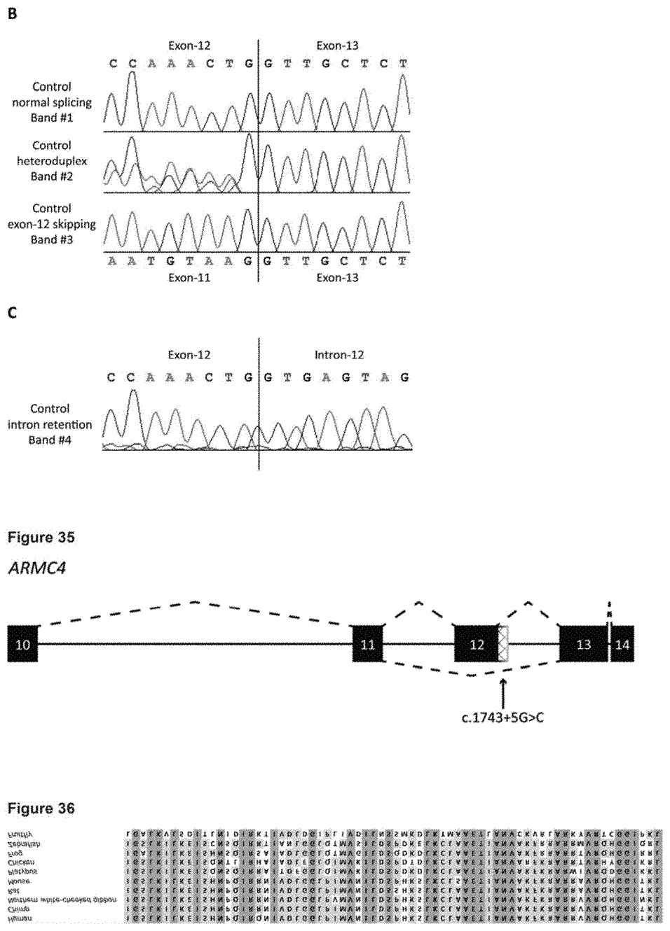

FIG. 35: Schematic of ARMC4 splicing and coordinates of the c.1743+5G>C variant. The predominant ARMC4 isoforms splice exon-10-11-12-13-14 sequentially.

### Consequences for the Encoded ARMC4 Protein:

We detect increased levels of ARMC4 exon-12 skipping relative to normal splicing of exons 11-12-13 in the parental carrier of the c.1743+5G>C variant, relative to controls. Exon-12 skipping removes 70 amino acids p. (Ile512 Leu581del) from the Armadillo domain of the ARMC4 protein, of which 30 residues are highly conserved between mammals, birds, fish, amphibians and insects. Evolutionary conservation of deleted residues within the Armadillo domain throughout vertebrate evolution strongly infer a functional importance.

FIG. 36. ARMC4 exon-12 amino acid conservation from mammals to fruitfly.

### Subject 6 AHI1

### Brief Clinical Summary Provided:

Joubert syndrome.

### Results of Previous Genetic Testing:

### AHI1 Chr6(GRCh37):g.135751015C>T

### AHI1 Chr6(GRCh37):g.135778732G>A

| | | | Disease | | Parent |
|---|---|---|---|---|---|
| Gene Name | Variant | Zygosity | (MIM) | Inheritance | Origin |
| AHI1 | c.2492 + 5G > A | Heterozygous | #608629 | Autosomal | Father |
| (NM_001134831.1) | | | Joubert | recessive | |
| | | | Syndrome 3; | | |
| AHI1 | c.1051C > T | Heterozygous | JBTS3 | | Mother |
| (NM_001134831.1) | | | | | |

### Nonsense Variant:

Previous genetic testing identified a nonsense variant in the AHI1 gene:

### Chr6(GRCh37):g.135778732G>A

### NM_001134831.1(AH11):c.1051C>T

### p. (Arg351*)

This variant has previously been reported in ClinVar (RCV000002087.3) as pathogenic.

### Extended Splice Site Variant:

Previous genetic testing identified an extended splice site variant in the AHI1 gene:

### Chr6(GRCh37):g.135751015C>T

### NM_001134831.1(AHI1):c.2492+5G>A

p.?

This variant has not previously been reported in ClinVar. This variant is not present in the Genome Aggregation Database (gnomAD).

mRNA Studies Performed to Assess the Extended Splice Site Variant:

RT-PCR was performed on mRNA extracted from the family trio (unaffected parents and affected individual). Several abnormally spliced products were observed in the patient (P) and paternal (F) samples (who carries who carries the c.2492+5G>A variant) using primers in exon 16 and exon 19. A band approximately 40 bp larger than expected, and another approximately 120 bp smaller than expected were observed in the patient and paternal samples.

No splicing defects were detected in the maternal sample (carrying the nonsense variant) using any primer combination.

Sanger sequencing revealed the c.2492+5G>A variant results in:
1. Skipping of exon 18.
2. The use of a cryptic donor splice site 40 bp downstream of the native exon 18 donor to retain 40 bp of intron 18 sequence. The use of this cryptic donor was predicted upon in silico analysis and encodes a premature termination codon. These transcripts are likely targeted by nonsense-mediated decay (NMD).

Abnormal splicing events were confirmed in two separate experiments using two different primer pairs.

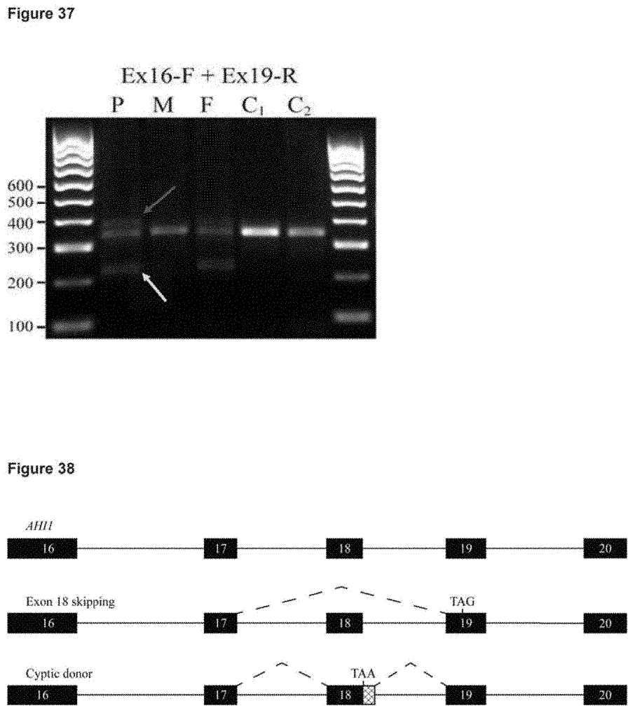

### FIG. 37

RT-PCR of AHI1 mRNA Isolated from Blood.

RT-PCR using primers in exons 16 and 19 of AHI1.

The c.2492+5G>A variant induces exon 18 skipping (yellow arrow) and use of a cryptic donor (red arrow).

Lanes: Patient (P), mother (M), father (F) control 1 (C₁), control 2 (C₂).

### Consequences for the Encoded AHI1 Protein:

Both the c.2492+5G>A and c.1051C>T variants induce premature termination codons with a clear, damaging effect for the encoded AHI1 protein. Both premature termination codons are predicted to target AHI1 transcripts for nonsense-mediated decay. Any AHI1 transcripts escaping nonsense-mediated decay encode AHI1 proteins lacking key functional domain(s) (WD domain(s) and SH3 domain) and are therefore likely to be dysfunctional or non-functional.

### Conclusions:

mRNA studies confirm the heterozygous c.2492+5G>A variant induces abnormal splicing of AHI1 transcripts. All splicing outcomes induce a premature termination codon and are unlikely to be translated into functional protein.

The heterozygous c.1051C>T nonsense variant has been previously reported as pathogenic in ClinVar.

Collective data from RT-PCR are consistent with likely pathogenicity of the AHI1c.2492+5G>A variant.

Compound heterozygous variants in AHI1 are consistent with autosomal recessive Joubert syndrome.

### Subject 7 (TAZ)

### Brief Clinical Summary Provided:

Neonate in intensive care with cardiac complications. Suspected Barth syndrome.

### Results of Previous Genetic Testing:

### TAZ ChrX(GRCh37):g.153640551G>C

| Gene Name | Variant | Zygosity | Disease (MIM) | Inheritance | Parent Origin |
|---|---|---|---|---|---|
| TAZ | c.238G > C | Hemizygous | #302060 Barth | X-linked | De novo |
| (NM_000116.3) | | | syndrome | recessive | |
| | | | #300394 Tafazzin | | |

Conclusions

1. mRNA studies confirm the hemizygous TAZ c.238G>C variant induces abnormal splicing of TAZ transcripts in blood and myocardial mRNA.
2. TAZ exon-2 is a canonical exon included in all predominant TAZ isoforms expressed in heart.
3. All detected abnormal splicing events are in-frame, though insert (use of intron-2 cryptic 5′ splice-site) or delete (exon-2 skipping) numerous amino acids within an evolutionarily conserved region of the tafazzin protein.
4. Abnormal splicing outcomes detected are consistent with a damaging effect for the encoded tafazzin protein.
cDNA Studies to Assess the Missense/5′ Splice-Site Variant (Last Base of Exon):

RT-PCR was performed on mRNA extracted from the affected individual.

### Splicing of TAZ is Complex (See FIG. 2).

TAZ exon-1 naturally uses two alternate 5′ splice-sites. The first exon-1 5′ splice-site is used most commonly.
TAZ exon-3 naturally uses multiple alternate donor splice sites. The first exon-3 5′ splice-site is used most commonly.
This gives rise to multiple products using primers in exons-1 and 4 flanking the exon-2 variant (see controls)

Summary of Results in Blood cDNA:
1. RNA studies of TAZcDNA derived from RNA derived from whole blood gave robust PCR results.
2. Exon-2 is a canonical exon within the predominant TAZ isoform in heart.
3. The c.238G>C p.Gly80Arg variant was not detected in the maternal sample by Sanger sequencing of PCR amplicons, indicating a de novo change in the patient.
4. TAZpre-mRNA splicing Exon 1-2-3-4 is normal in the maternal cDNA, and normal in cDNA derived from whole blood from four controls (two male controls aged 3 yrs and adult; two female controls, adult).
5. We find no evidence for normal splicing of Exon 1-2-3-4 in TAZ mRNA in the affected neonate, using 5 different primer combinations. FIG. 1 Gel B: absent band using a forward primer in exon-1 (5′UTR-F) and reverse primer in exon-2 (Ex2-R).
6. We detect two predominant abnormal splicing events (FIG. 1 Gel A):
   a. Band #1. Use of an Intron-2 cryptic 5′ splice-site. Abnormally includes 36 nt of intron-2 into the TAZpre-mRNA.
   b. Band #2. Exon-2 skipping. Abnormally removes 129 nucleotides from the TAZ pre-mRNA.

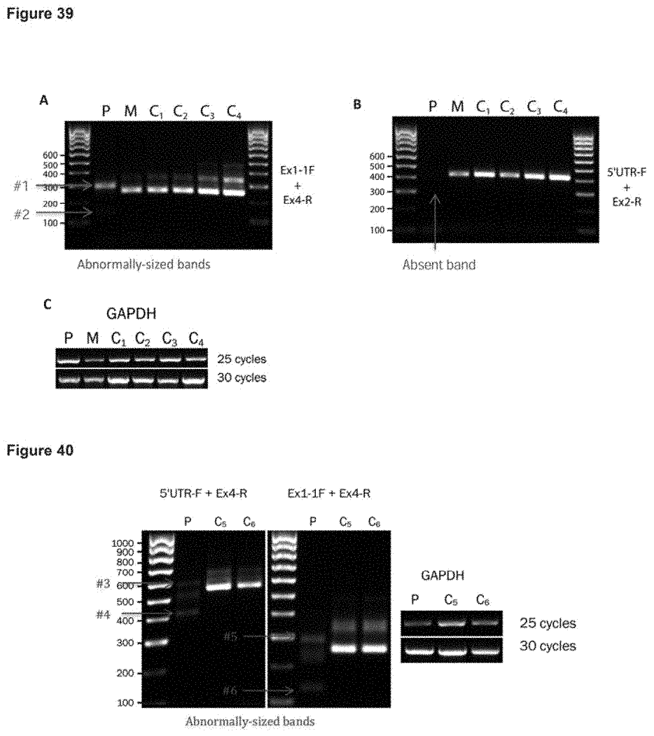

FIG. 39: RT-PCR of TAZ mRNA isolated from blood. A) Several abnormally sized bands were detected in the patient sample (P), relative to four control samples (C₁-C₄). No normally spliced products were detected in the patient sample (P) using a forward primer in exon-1 and a reverse primer in exon-4 of TAZ. B) No product was detected in the patient sample (P) using a forward primer in the 5′UTR and a reverse primer in exon-2 of TAZ, indicating exon-2 spliced into the TAZ at very low levels (exon-2 skipping). C) Amplification of GAPDH demonstrates similar cDNA loading. Lanes: Patient (P), mother (M), father (F) control 1 (C₁) (male, 4 years), control 2 (C₂) (male, 38 years), control 3 (C₃) (female, adult), control 4 (C₄) (female, 43 years).

Summary of Results in Myocardial cDNA:

RT-PCR was performed on mRNA extracted from the myocardium of the affected individual and two disease controls (C₅, C₆).
1. RNA studies of TAZcDNA derived from RNA derived from myocardium gave robust PCR results.
2. TAZpre-mRNA splicing Exon 1-2-3-4 is normal in myocardial cDNA samples from two disease controls.
3. We detect two predominant abnormal splicing events (FIG. 2):
   a. Band #3 and #5. Use of an Intron-2 cryptic 5′ splice-site. Abnormally includes 36 nt of intron-2 into the TAZpre-mRNA.
   b. Band #4 and #6. Exon-2 skipping. Abnormally removes 129 nucleotides from the TAZ pre-mRNA.

FIG. 40: RT-PCR of TAZ mRNA isolated from myocardium. Several abnormally sized bands were detected in the patient sample (P), relative to two disease control samples (C₅, C₆). No normally spliced products were detected in the patient sample (P) using forward primers in the 5′UTR and exon-1, and a reverse primer in exon-4 of TAZ. Amplification of GAPDH demonstrates similar cDNA loading. Lanes: Patient (P), control 5 (C₅) (32 years), control 6 (C₆) (female, 10 years).

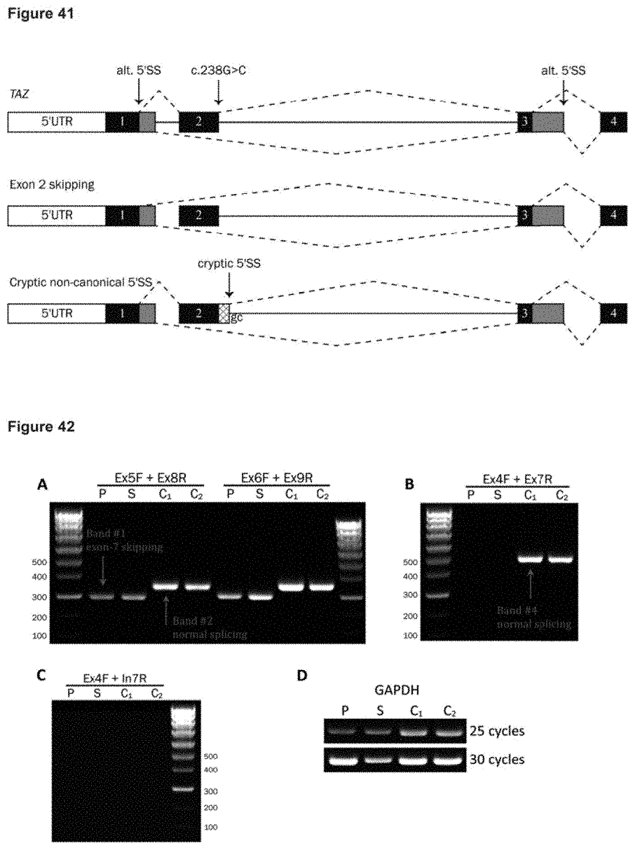

FIG. 41: Schematic of the splicing abnormalities induced by the c.238G>C variant.

### Consequences for the Encoded TAZ Protein:

Use of Intron-2 cryptic 5′ splice-site abnormally includes 36 nt of intron-2 into the TAZ pre-mRNA, encoding 12 ectopic amino acids into the tafazzin protein.

Exon-2 skipping abnormally removes 129 nucleotides from the TAZ pre-mRNA. This event is in frame, deleting 43 (highly conserved) amino acids from the encoded tafazzin protein.

The RT-PCR results infer splicing outcomes consistent with a damaging effect for the encoded tafazzin protein.

### Subject 8 (LAMP2)

### Brief Clinical Summary Provided:

Severe concentric hypertrophic cardiomyopathy. Proximal muscle weakness with a raised CK level.

### Results of Previous Genetic Testing:

Previous genetic testing identified a hemizygous variant of uncertain significance in LAMP2:

ChrX(GRCh37):g.119576451T>A

### NM_013995.2(LAMP2):c.928+3A>T

This variant has not previously been reported in ClinVar. This variant is not present in the Genome Aggregation Database (gnomAD).

### LAMP2 ChrX/(GRCh37):g.119576451T>A

| | | | | | Parent |
|---|---|---|---|---|---|
| Gene Name | Variant | Zygosity | Disease (MIM) | Inheritance | Origin |
| LAMP2 | c.928 + 3A > T | Hemizygous | #300257 | X-linked | Not |
| (NM_013995.2) | | | Danon disease | dominant | determined |

Conclusions
1. mRNA studies confirm the hemizygous LAMP2: c.928+3A>T variant induces abnormal splicing of LAMP2 transcripts in blood mRNA.
2. LAMP2 transcripts expressed in the proband and affected sibling show exon-7 skipping (p.Lys289Phefs*36). This abnormal splicing event is not observed in controls and induces a frameshift that encodes a premature termination codon, with clear damaging consequences for the encoded LAMP2 protein.
3. We were unable to find evidence for residual, normal splicing of LAMP2 exons 6-7-8 in the proband or affected sibling. Therefore, normally spliced LAMP2 transcripts are below the level of PCR detection, or absent.
4. LAMP2 exon-7 is a canonical exon included in all LAMP2 isoforms expressed in brain, myocardium, skeletal muscle and blood. Therefore splicing outcomes observed in blood mRNA hold relevance to the predominant LAMP2 isoforms in the manifesting tissues.

The most likely outcome for the encoded LAMP2 protein is protein deficiency, due to nonsense mediated decay of mis-spliced transcripts that will preclude translation of LAMP2 protein. A possible outcome is expression of a truncated, dysfunctional LAMP2 (which lack a transmembrane anchor) through translation of mis-spliced LAMP2 transcripts that escape nonsense-mediated decay.

mRNA Studies Performed to Assess the Extended Splice Site Variants:
Summary of Results in mRNA Derived from Whole Blood

RT-PCR was performed on mRNA extracted from the whole blood of the proband and affected male sibling.

We detect one abnormal splicing event resulting from the c.928+3A>T variant (FIG. 42):

1. Exon-7 skipping (FIG. 2; Band #1)

We did not detect normal splicing of LAMP2 transcripts in the proband and affected sibling (FIG. 42B).

FIG. 42: RT-PCR of LAMP2 mRNA isolated from blood.

A) Using two sets of primers flanking the c.928+3A>T variant we detect a single band corresponding to exon-7 skipping in the proband and affected sibling mRNA (Band #1). In two controls we detect a single band corresponding to normal exon-6-7-8-splicing (Band #2).
B) Using a forward primer in exon-4 and a reverse primer in exon-7 we are unable to detect any transcripts containing exon-7 in the proband or affected sibling.
C) Using a reverse primer in intron-7, designed to detect use of a potential cryptic 5′ splice site upstream of the native exon-7 5′ splice site, we found no evidence of abnormal splicing.
D) Amplification of GAPDH demonstrates cDNA loading. Lanes: Proband (P), Sibling (S) (male, 3 years), Control 1 (C₁) (male, 7 months), Control 2 (C₂) (male, 5 years). Replicate samples were subject to PCR for 25 or 30 cycles in order to confirm the PCR cycling conditions were sub-saturating and able to detect lower levels or quality of a specimen.

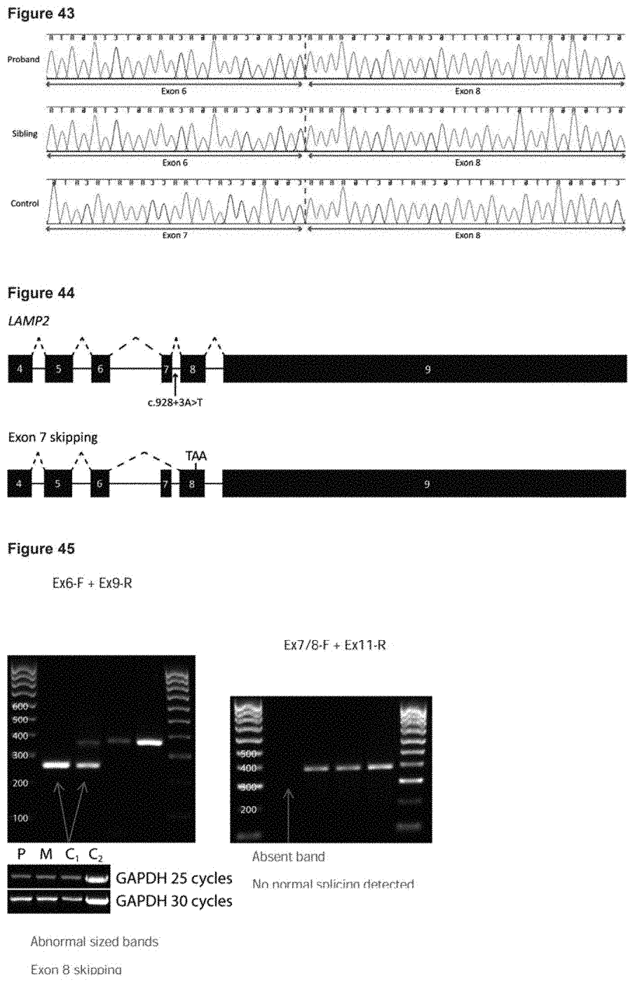

FIG. 43 Sanger sequencing of RT-PCR amplicons. Sequencing showed the abnormal sized Band #1 (FIG. 2A) in the proband and sibling samples was due to exon-7 skipping.

FIG. 44: Schematic of splicing abnormality induced by the c.928+3A>T variant.

### Consequences for the Encoded LAMP2 Protein:

The c.928+3A>T variant induces exon-7 skipping (p.Lys289Phefs*36) causing a frameshift and encoding premature termination codon. These mis-spliced transcripts are predicted to be targeted for nonsense-mediated decay. Any LAMP2 transcripts escaping nonsense-mediated decay encode LAMP2 proteins lacking the C-terminal transmembrane domain and are likely to be dysfunctional/non-functional.

### Subject 9 (OPHN1)

### Brief Clinical Summary Provided:

Mental Retardation, ataxia, distinct facial features.

### Results of Previous Genetic Testing:

Previous genetic testing identified a variant of uncertain significance in the OPHN1 gene:

### ChrX(GRCh37):g.67431946T>C

### NM_002547.2(OPHN1):c.702+4A>G

p.?

This variant has not previously been reported in ClinVar. This variant is not present in the Genome Aggregation Database (gnomAD).

### OPHN1 ChrX(GRCh37):g.67431946T>C

| Gene Name | Variant | Zygosity | Disease (MIM) | Inheritance | Parent Origin |
|---|---|---|---|---|---|
| OPHN1 | c.702 + 4A > G | Hemizygous | #300486 | X-linked | Mother |
| (NM_002457.2) | | | Mental | recessive | |
| | | | retardation, X- | | |
| | | | linked, with | | |
| | | | cerebellar | | |
| | | | hypoplasia and | | |
| | | | distinctive facial | | |
| | | | appearance | | |

mRNA Studies Performed to Assess the Extended Splice Site Variant:

RT-PCR was performed on mRNA extracted from the whole blood of the affected individual and his unaffected mother

FIG. 45. RT-PCR of OPHN1 mRNA isolated from blood. A) Abnormally sized bands were detected in the patient and maternal samples relative to two control samples. B) No product was detected in the patient sample using a forward primer bridging the exon-7/exon-8 junction to specifically probe for normally spliced transcripts. C) Amplification of GAPDH demonstrates similar cDNA loading. Lanes: Patient (P), mother (M), control 1 (C₁) (male, 5 years), control 2 (C₂) (female, 26 years).

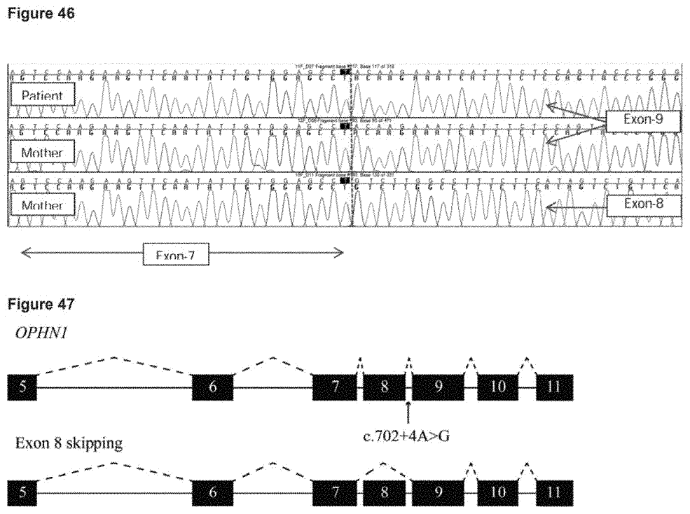

No evidence for normal splicing in the patient sample was identified (FIG. 45) using three different primer combinations (not shown, data available upon request). We detect one predominant abnormal splicing event—exon-8 skipping that removes 105 nucleotides from the OPHN1 pre-mRNA (FIG. 1 Gel A, FIG. 46, FIG. 47).

FIG. 46. Sanger sequencing of RT-PCR amplicons confirmed the abnormal sized bands in the patient and mother samples were due to exon-8 skipping. Normally spliced OPHN1 transcripts were also detected in the maternal sample.

FIG. 47: Schematic of exon-8 skipping induced by the c.702+4A>G variant.

### Consequences for the Encoded OPHN1 Protein:

Exon-8 skipping abnormally removes 105 nucleotides from the OPHN1 pre-mRNA. This event is in frame, deleting 35 amino acids p. (Val200_Asn234del) from the encoded OPHN1 protein.

Our RT-PCR results infer splicing outcomes consistent with a damaging effect for the encoded Oligophrenin-1 protein.

Conclusions:
1. mRNA studies confirm the hemizygous OPHN1 c.702+4A>G variant induces abnormal splicing of OPHN1 transcripts in blood mRNA.
2. OPHN1 exon-8 is a canonical exon included in all predominant OPHN1 isoforms expressed in brain.
3. The absence of this variant from gnomAD is consistent with a rare X-linked recessive disorder.
4. Exon 8 skipping induced by the OPHN1 c.702+4A>G variant abnormally removes 35 amino acids from the encoded Oligophrenin-1 protein.

Hemizygous variants in OPHN1 are consistent with X-linked recessive mental retardation MIM #300486

### Subject 10 (HSD17B4)

### Brief Clinical Summary Provided:

Perrault syndrome.

### Results of Previous Genetic Testing:

A clinical exome analysis identified two heterozygous variants in HSD17B4:

Pathogenic Missense Variant

### Chr5(GRCh37):g.118788316G>A

### NM_000414.3(HSD17B4):c.46G>A

### p. (Gly16Ser)

Previously reported as likely pathogenic/pathogenic in ClinVar (RCV000415821.5, RCV000008094.5, RCV000688945.1). This variant is present in the Genome Aggregation Database (gnomAD) at an allele frequency of 0.0002025 (57/281472).

Variant of Uncertain Significance

### Chr5(GRCh37):g.118842585G>C

### NM_000414.3(HSD17B4):c.1333+1G>C

p.?

This variant has no previous reports in ClinVar. This Variant is absent from the Genome Aggregation Database (gnomAD).

### HSD17B4: Chr5(GRCh37):g.118788316G>A

### HSD17B4: Chr5(GRCh37):g.118842585G>C

| | | | | | Parent |
|---|---|---|---|---|---|
| Gene Name | Variant | Zygosity | Disease (MIM) | Inheritance | Origin |
| HSD17B4 | c.46G > A | Heterozygous | #233400 | Autosomal | Not provided |
| (NM_000414.3) | | | | recessive | |
| | c.1333 + 1G > C | Heterozygous | Perrault | | Not provided |
| | | | Syndrome 1 | | |

Conclusions
1. Messenger RNA studies confirm the c.1333+1G>C variant induces abnormal splicing of HSD1764.
2. We detect one predominant abnormal splicing event, exon-15 skipping. This is an in-frame event that removes 24 amino acids (p.Gly421_Asp444del) from the Enoyl-CoA hydratase 2 region of the HSD17B4 protein.
   mRNA derived from blood and fibroblasts were used as controls
   mRNA Studies Performed to Assess the c.1333+1G>C Variant:

RT-PCR was performed on mRNA extracted from a transformed lymphoblast cell line derived from the affected individual.
We detect one predominant abnormal splicing event, exon-15 skipping. c.1262_1333del (FIG. 2 A-C). This event is in-frame, removing 24 amino acids (p.Gly421_Asp444del) from the Hydroxysteroid (17-beta) dehydrogenase 4 protein.
We also detect normal exon-14-exon-15-exon-16 splicing in the patient that is likely derived from the second HSD17B4 allele (FIG. 2 A-C).
The patient lymphoblast cells were also cultured in the presence of cycloheximide (CHX), a nonsense-mediated mRNA decay (NMD) inhibitor, in order to detect splicing outcomes targeted by NMD. This did not reveal further abnormal splicing events (FIGS. 2 B & C).

In the absence of appropriate lymphoblast cell control RNA samples, we used mRNA from peripheral blood mononuclear cells (PBMCs) and primary human fibroblasts (PHF) as controls. It must be noted that HSD17B4 transcripts may be spliced differently between these tissues and consequently mRNA studies from PBMCs and fibroblasts may not accurately reflect splicing in the transformed lymphoblast cell line from the proband.

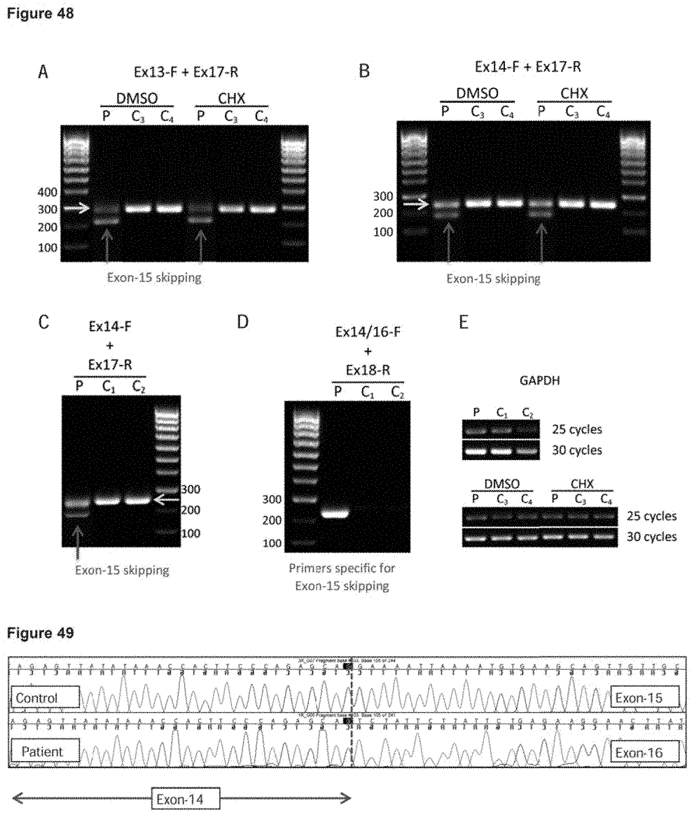

FIG. 48. RT-PCR of HSD17B4 mRNA isolated from patient lymphoblasts. A)-C) Primers flanking the c.1333+1G>C variant amplified an abnormal lower band in the patient sample (red arrows). Sanger sequencing confirmed these amplicons correspond with exon-15 skipping. Yellow arrows: RT-PCR amplicon with normal exon-14-exon-15-exon-16 splicing was also detected in patient RNA, confirmed by Sanger sequencing, and presumably derived from the HSD17B4 allele bearing the c.46G>A variant. D) Using a forward primer (Ex14/16-F) designed to anneal with the exon-14-exon-16 junction we were able to specifically amplify HSD17B4 transcripts that skipped exon-15. Levels of exon-15 skipping are notably higher in the patient mRNA relative to two controls. E) GAPDH demonstrates similar cDNA loading. Lanes: Patient (P), control 1 (C₁) (PBMC mRNA, female, 43 years), control 2 (C₂) (PBMC mRNA, female, 37 years), control 3 (C₃) (PHF mRNA, female, 7 years), control 4 (C₄) (PHF mRNA, female, 53 years).

FIG. 49. Sanger sequencing of RT-PCR amplicons confirm exon-15 skipping in HSD17B4 transcripts of the patient mRNA.

### Consequences for the Encoded HSD17B4 Protein

The c.1333+1G>C variant induces exon-15 skipping in HSD17B4 transcripts. This is an in-frame event which removes 24 amino acids (p.Gly421 Asp444del) from the Enoyl-CoA hydratase 2 region of the Hydroxysteroid (17-beta) dehydrogenase 4 protein.

### Subject 11 (ACE)

### Brief Clinical Summary Provided:

In-utero death and post mortem revealed renal tubular dysgenesis.

### Results of Previous Genetic Testing:

Sequencing of ACE identified a homozygous variant of uncertain significance:

Chr17:g.61561337G>C

### NM_000789.3:c.1709+5G>C

This variant has not previously been reported in ClinVar. This variant is not present in the Genome Aggregation Database (gnomAD).

### ACE Chr17(GRCh37):g.61561337G>C

| | | | | | Parent |
|---|---|---|---|---|---|
| Gene Name | Variant | Zygosity | Disease (MIM) | Inheritance | Origin |
| ACE | c.1709 + 5G > C | Homozygous | #267430 Renal | Autosomal | Parents both |
| (NM_000789.3) | | | Tubular | Recessive | confirmed |
| | | | Dysgenesis; | | unaffected |
| | | | RTD | | carriers |

Conclusions
1. RNA studies confirm the ACE c.1709+5G>C variant induces abnormal splicing of ACE transcripts in blood mRNA.
2. We detect two abnormal splicing events:
   a. In-frame exon 11 skipping. This event removes 41 amino acids from the peptidase M2 domain of ACE, among which 26 residues are conserved from mammals to fish.
   b. Use of a cryptic 5′-splice site which induces a frameshift and encodes a premature termination codon p. (Ala565Glufs*64). These transcripts are predicted to be degraded by nonsense mediated decay. Any ACE transcripts escaping nonsense-mediated decay encode a truncated ACE protein lacking 741 amino acids from the C-terminus
3. ACE exon 11 is a canonical exon in all long isoforms of ACE expressed in kidney, blood, fibroblasts and renal epithelia. Therefore splicing outcomes observed in blood, fibroblasts and renal epithelia mRNA hold relevance to the long ACE isoform(s) in the manifesting tissue (kidney).
4. The short testis-specific isoform of ACE uses an alternative promoter in intron 12, downstream of the c.1709+5G>C variant, and is therefore unlikely to be affected.
   mRNA Studies Performed to Assess the Extended Splice Site Variants:
   Summary of Results in mRNA Derived from Blood

RT-PCR was performed on mRNA extracted from the whole blood of the unaffected parent carriers.

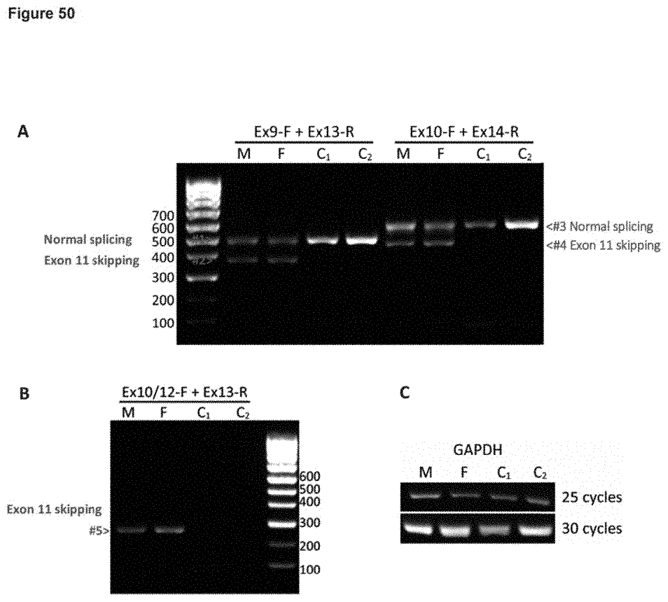

We detect one abnormal splicing event resulting from the c.1709+5G>C variant (FIG. 50):

1. Exon 11 skipping (Bands #2, #4).

FIG. 50 RT-PCR of ACE mRNA isolated from whole blood.

A) Using primers flanking the c.1709+5G>C variant we detected 2 bands:
Band #1 and Band #3: normally spliced ACE transcripts
Band #2 and Band #4: exon 11 skipping (only detected in the maternal and paternal samples).
B) We used a forward primer designed to anneal with the exon 10-exon 12 junction to specifically amplify ACE transcripts with exon 11 skipping. Exon 11 skipping was only observed in the maternal and paternal mRNA samples (Band #5), and was not detected in two controls.
C) Amplification of GAPDH demonstrates cDNA loading. Lanes: Mother (M), Father (F), Control 1 (C₁) (Female, 36 years), Control 2 (C₂) (Male, 39 years).

We also detect normal splicing of ACE transcripts in the maternal and paternal samples.

We used a reverse primer in intron 11 to specifically amplify ACE transcripts with intron 11 retention. There were no detectable levels of intron 11 retention in all samples (data not shown, available on request).

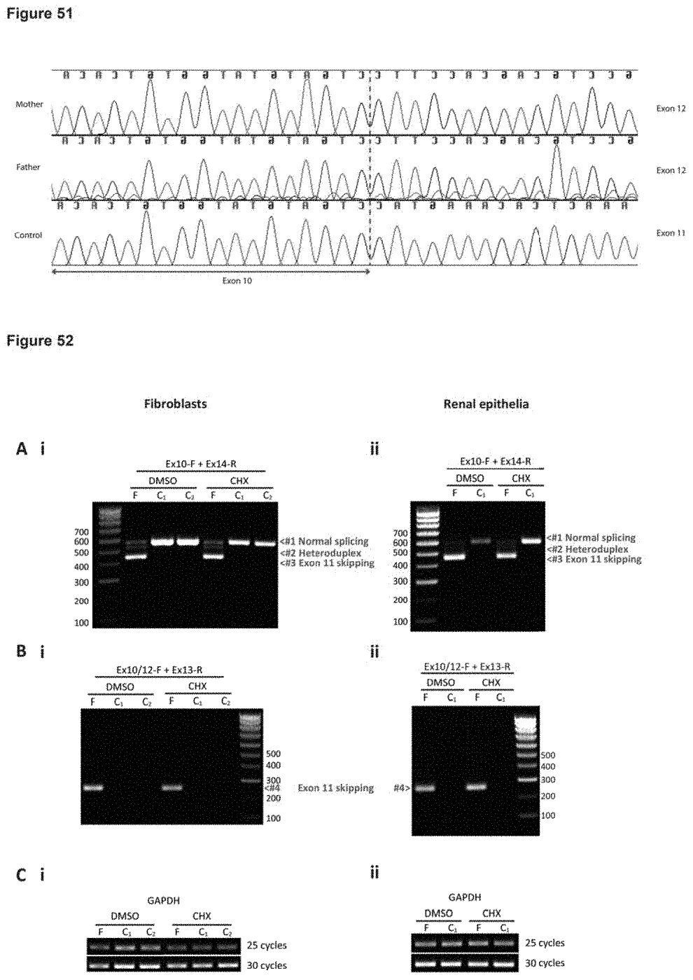

FIG. 51: Sanger sequencing of RT-PCR amplicons. Sequencing showed the abnormally sized Band #2 (FIG. 2A) in the maternal and paternal samples was due to exon 11 skipping.

Summary of Results in mRNA Derived from Fibroblasts and Renal Epithelial Cells

RT-PCR was performed on mRNA extracted from the skin fibroblasts and renal epithelia of the unaffected father.

The fibroblasts and renal epithelial cells were cultured in the presence of cycloheximide (CHX), a nonsense-mediated mRNA decay (NMD) inhibitor, or DMSO (control), in order to detect splicing outcomes targeted by NMD.

We detect three different splicing events in both cell types:
1. Normal splicing (Band #1)
2. Heteroduplex amplicon (Band #2)
   a. This band contains a mix of normally spliced transcripts and exon 11 skipping in DMSO control conditions.
   b. An additional abnormal splicing event is detected after CHX treatment. Use of a cryptic ‘GC’ 5′-splice site induces a frameshift and encodes a premature termination codon p. (Ala565Glufs*64). These transcripts are predicted to be degraded by NMD and are rescued by CHX treatment.

In-frame exon 11 skipping (Band #3, #4)

FIG. 52

RT-PCR of ACE mRNA isolated from fibroblasts (i) and renal epithelia (ii).
A) Using primers flanking the c.1709+5G>C variant we detected three bands:
Band #1: normally spliced ACE transcripts (paternal sample and controls)
Band #2 Heteroduplex amplicon (paternal sample only)

DSMO: contains a mix of normally spliced transcripts and exon 11 skipping

CHX: contains normally spliced transcripts, exon 11 skipping and use of a cryptic 5′-splice site

Band #3: exon 11 skipping (only detected in the paternal sample).
B) We used a forward primer designed to anneal with the exon 10-exon 12 junction to specifically amplify ACE transcripts with exon 11 skipping. Exon 11 skipping was only observed in the paternal mRNA samples (Band #4), and was not detected in two controls.
C) Amplification of GAPDH demonstrates cDNA loading. Lanes:
i) Father (F), Control 1 (C₁) (Male, 52 years), Control 2 (C₂) (Male, 49 years).
ii) Father (F), Control 1 (C₁) (Male, 30 years).

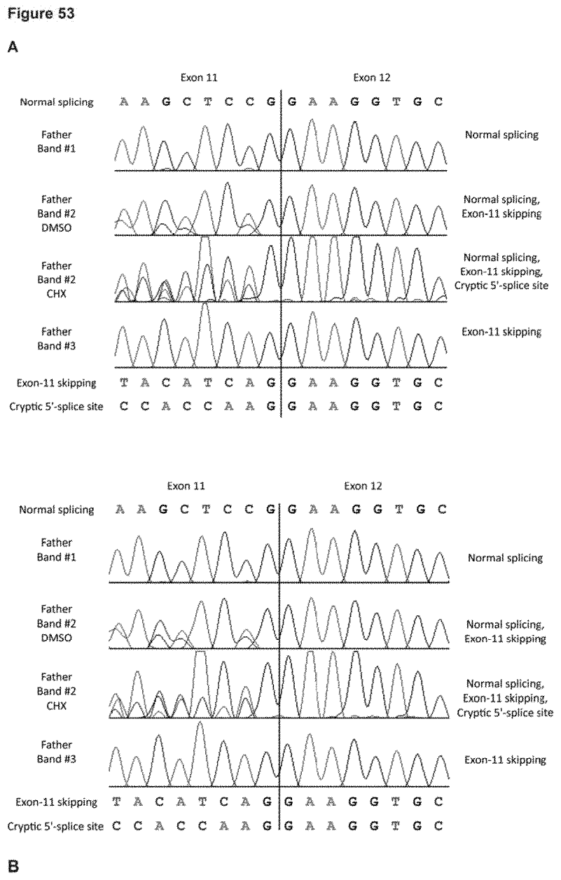

FIG. 53 Sanger sequencing of RT-PCR amplicons from fibroblasts (A) and renal epithelia (B).

Band #1 contains normally spliced exon 10-11-12 transcripts (DMSO and CHX).
Band #2 DMSO: heteroduplex containing both normally spliced transcripts and exon 11 skipping.

CHX: heteroduplex containing normally spliced transcripts, exon 11 skipping and use of

a cryptic ‘GC’ 5′-splice site.

Band #3 contains transcripts with exon 11 skipping (DMSO and CHX).

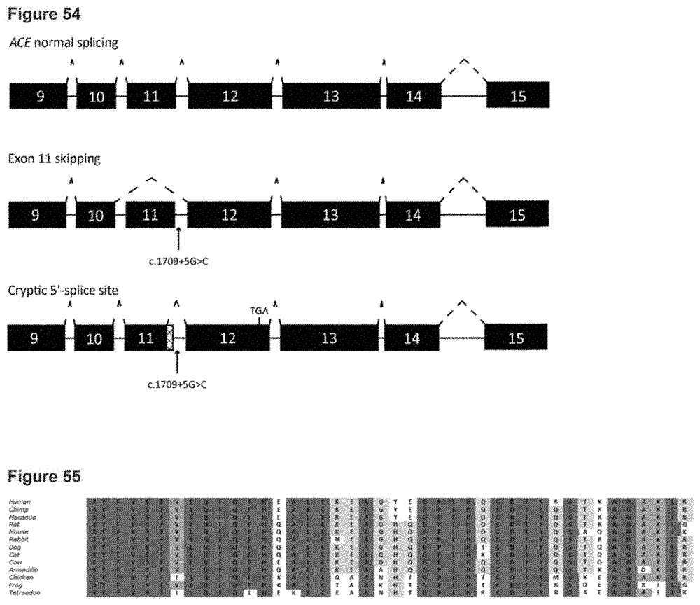

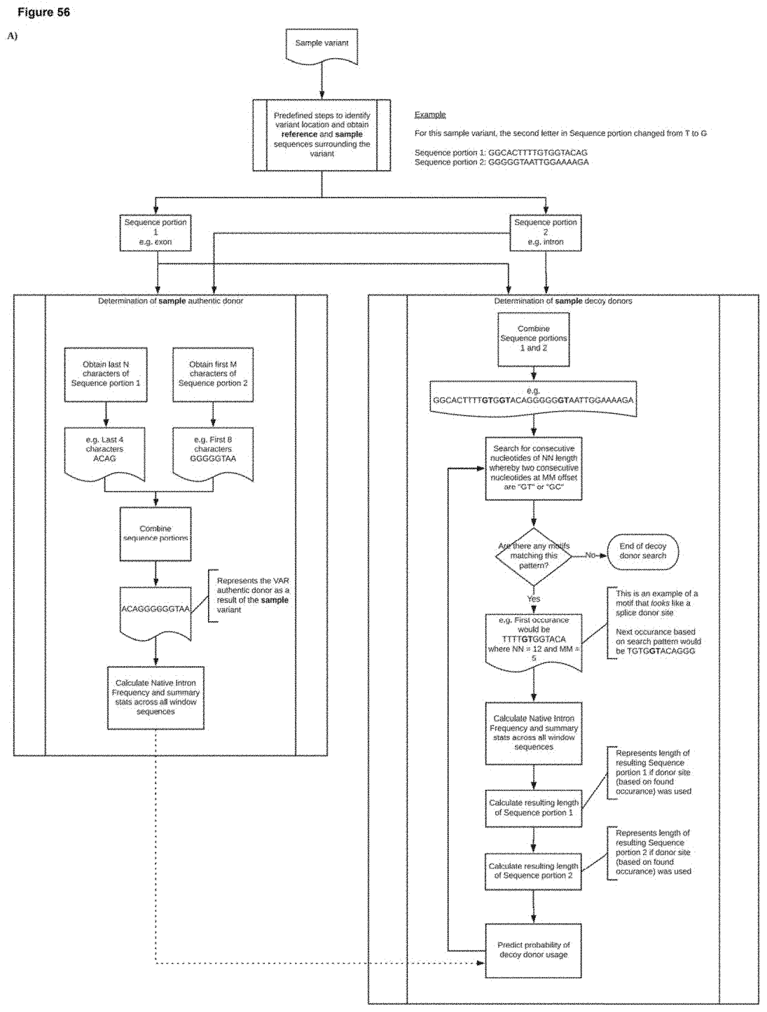

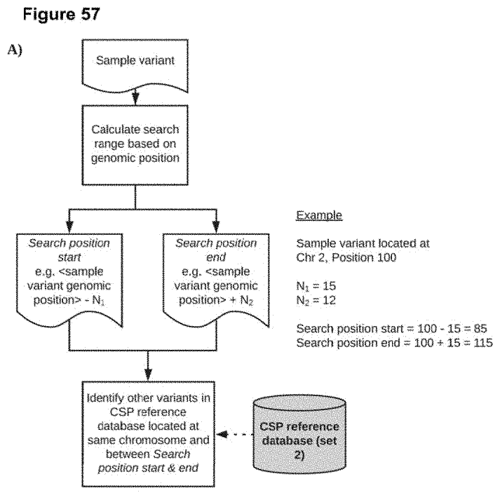

FIG. 54: Schematic of splicing abnormalities induced by the c.1709+5G>C variant.

### Consequences for the Encoded ACE Protein:

The c.1709+5G>C variant results in:

1. Exon 11 skipping, an in-frame event

2. Use of a cryptic 5′-splice site, out-of-frame

Exon 11 skipping removes 41 amino acids p. (Tyr530_Arg570del) from the peptidase M2 domain of ACE, of which 26 residues are highly conserved between mammals, birds, amphibians and fish (FIG. 55). Loss of 26 highly conserved residues is likely to exert a damaging effect for the encoded ACE protein.

Use of the cryptic ‘GC’ 5′-splice site induces a frameshift and encodes a premature termination codon p. (Ala565Glufs*64). These transcripts are predicted to be degraded by NMD, consistent with rescue of these transcripts upon CHX treatment. Any transcripts escaping NMD will result in the loss of the 741 C-terminal residues of ACE, with likely/clear damaging consequences

FIG. 55 ACE exon 11 amino acid conservation between mammals, birds, amphibians and fish.

1. A method of identifying an abnormal splice site in a sample splice site from a subject, said method comprising
(a) obtaining a first sample splice site sequence comprised in the sample splice site from the subject; and
(b) determining the frequency at which the sequence occurs in a reference genome, expressed as a Native Intron Frequency of the first sample splice site sequence (NIFᵥₐᵣ₋₁);
wherein a NIFᵥₐᵣ₋₁ of 0 (zero) indicates that the sample splice site is abnormal.

2. The method of claim 1, wherein the method is repeated with one or more sample splice site sequences comprised in the sample splice site, wherein each sample splice site sequence comprises non-identical, consecutive nucleotides of the sample splice site, and wherein a NIFᵥₐᵣ₋₁ of 0 (zero) for any sample splice site sequence indicates that the sample splice site is abnormal.

3. The method of claim 1, said method comprising:
(a) obtaining a first sample splice site sequence comprised in the sample splice site from the subject;
(b) determining a measure of Native Intron Frequency of the first sample splice site sequence (NIFᵥₐᵣ₋₁);
(c) determining a measure of Native Intron Frequency of a first reference splice site sequence (NIF_{ref-1}); wherein the first reference splice site sequence and the first sample splice site sequence each originate from the same corresponding region of a gene; and
(d) determining a risk of abnormal splicing for the sample splice site by comparing NIFᵥₐᵣ₋₁ with NIF_{ref-1} against a Clinical Splice Predictor (CSP) reference database.

4. The method of claim 1, said method comprising:
(a) obtaining a first sample splice site sequence comprised in the sample splice site from the subject;
(b) determining a measure of Native Intron Frequency of the first sample splice site sequence (NIF_{var 1});
(c) determining a measure of Native intron Frequency of a first reference splice site sequence (NIF_{ref 1}); wherein the first reference splice site sequence and the first sample splice site sequence each originate from the same corresponding region of a gene; and
(d) determining a risk of abnormal splicing for the sample splice site by comparing NIF_{var 1} with NIF_{ref 1} against a Clinical Splice Predictor (CSP) reference database;
wherein the method steps (a) to (c) are repeated with one or more sample splice site sequences comprised in the sample splice site, wherein each sample splice site sequence comprises non-identical nucleotides of the sample splice site, and wherein step (d) further includes a comparison of each further NIFᵥₐᵣ with each corresponding NIF_{ref} against a CSP reference database.

5. The method of claim 1 further comprising:
(a) determining a Percentile (NIFᵥₐᵣ-i) of the first sample splice site sequence;
(b) determining a measure of Native Intron Frequency of a first reference splice site sequence (NIF_{ref-1}); wherein the first reference splice site sequence and the first sample splice site sequence each originate from the same corresponding region of a gene;
(c) determining a Percentile (NIF_{ref-1}) of the first reference splice site sequence; and
(d) determining a risk of abnormal splicing for the sample splice site by comparing Percentile (NIFᵥₐᵣ₋₁) with Percentile (NIF_{ref-1}) against a CSP reference database.

6. The method of claim 1, further comprising
(c) determining a Percentile (NIF_{var 1}) of the first sample splice site sequence;
(d) determining a measure of Native Intron Frequency of a first reference splice site, sequence (NIF_{ref 1}); wherein the first reference splice site sequence and the first sample splice si sequence each originate from the same corresponding region of a gene;
(e) determining a Percentile (NIF_{ref 1}) of the first reference splice site sequence; and
(f) determining a risk of abnormal splicing for the sample splice site by comparing Percentile (NIF_{var 1}) with Percentile NIF_{ref 1}) against a CSP reference database;
and wherein the method steps (a) to (e) are repeated with one or more sample splice site sequences comprised in the sample splice site, wherein each sample splice site sequence comprises non-identical, consecutive nucleotides of the sample splice site, and wherein step (f) further includes a comparison of each further Percentile (NIFᵥₐᵣ) and each corresponding Percentile (NIF_{ref}) against a CSP reference database.

7-9. (canceled)

10. The method of identifying claim 1, said method further comprising:
(c) determining a clinical classification (s) associated with the nucleotide sequence of the first sample splice site sequence;
(d) determining a risk of abnormal splicing for the sample splice site by assessing the clinical classification(s) associated with the nucleotide sequence of the first sample splice site sequence determined in step (c) against a CSP reference database.

11. The method of claim 1, further comprising
(c) determining a clinical classifications) associated with the nucleotide sequence of the first sample splice site sequence;
(d) determining a risk of abnormal splicing for the sample splice site by assessing the clinical classification(s) associated with the nucleotide sequence of the first sample splice site sequence in step Cc) against a CSP reference database;
wherein steps (a) and (c) are repeated with one or more sample splice site sequences, wherein each sample splice site sequence comprises non-identical, consecutive nucleotides of the sample splice site,
and wherein step (d) comprising determining a risk of abnormal splice of the sample splice site by assessing the clinical classifications of each nucleotide sequence of each sample splice site sequence determined in (c) identified as sample splice sites of other subjects in the CSP reference database;
and wherein the classified sample splice sites of other subjects in the CSP reference database have the identical nucleotide sequence as the sample splice site sequence in the test subject but localise to a different exon-intron junction.

12. A method of identifying an abnormal splice site in a sample splice site from a subject, said method comprising:
(a) obtaining a first sample splice site sequence comprised in the sample splice from the subject;
(b) determining a measure of Native Intron Frequency of the first pc splice site sequence (NIFᵥₐᵣ₋₁);
(c) determining a Percentile (NIFvar 1) of the first sample splice site sequence and determining a Percentile (NIFref 1) of the first reference splice site sequence;
(d) determining a measure of Native Intron Frequency of a first reference splice site sequence (NIF_{ref-1}); wherein the first reference splice site sequence and the first sample splice site sequence originate from the same corresponding region of a gene;
(e) calculating a lower bound and an upper bound for Percentile (NIFᵥₐᵣ₋₁) and calculating a lower bound and an upper bound for Percentile (NIF_{ref-1});
(f) determining a range of NIF-shift by comparing the lower and upper bounds for NIFᵥₐᵣ₋₁ with the lower and upper bounds for NIF_{ref-1} calculated in (e);
(f) identifying unique variant(s) in the CSP database that create the identical nucleotide sequence of one or more sample splice sites from the subject (var-x): wherein the identical sample splice sites identified in other subjects in the CSP database localise to a different splice site at a different exon-intron junction to the sample splice site in the test subject;
(g) repeating steps (b-f) to calculate the NIF-shift for all non-identical, consecutive nucleotide sequences of the sample splice site in the CSP database identified in (f)
(h) determining a clinical classification associated with each identical var-x nucleotide sequence in the sample splice site identified in the CSP database in (g);
(i) determining the risk of abnormal splicing or likelihood of maintaining splicing for the sample splice site in the subject by assessing the clinical classification determined in step (h) of each identical var-x nucleotide sequence in a sample splice site in the CSP database.

13-15. (canceled)

16. The method of claim 12 splice site in a sample splice site from a subject, said method further comprising:
(a′) determining a clinical classification(s) associated with the nucleotide sequence of the first sample splice site sequence;
(c′) optionally determining a clinical classification(s) associated with the nucleotide sequence of the first reference splice site sequence; and
wherein the determining the risk of abnormal splicing for the sample splice site comprises (1 comparing the NIFᵥₐᵣ₋₁ with the NIF_{ref-1} against a CSP reference database, (2) assessing the clinical classification(s) associated with the nucleotide sequence of the first sample splice site sequence determined in step (a) and, the clinical classification(s) associated with the nucleotide sequence of the first reference splice site sequence optionally determined in step (c); and (3) assessing the clinical classification determined in step (g) for each similar NW-shift variant identified in step (h).

17. (canceled)

18. The method of claim 1, comprising:
(a) obtaining a first sample splice site sequence comprised in the sample splice site from the subject;
(b) determining a measure of Native Intron Frequency of the first sample splice site sequence (NIFᵥₐᵣ₋₁);
(c) determining a Percentile (NIFᵥₐᵣ₋₁) of the first sample splice site sequence;
(d) determining a measure of Native Intron Frequency of a first reference splice site sequence (NIF_{ref-1}); wherein the first reference splice site sequence and the first sample splice site sequence each originate from the same corresponding region of a gene;
(e) calculating a lower bound and an upper bound for Percentile (NIFᵥₐᵣ₋₁) and calculating a lower bound and an upper bound for Percentile (NIF_{ref-1});
(f) determining a range of NIF-shift by comparing the lower and upper bounds for Percentile (NIFᵥₐᵣ₋₁) with the lower and upper bounds for Percentile (NIF_{ref-1}) calculated in (h);
(g) identifying unique variants in the CSP database that affect the same splice site as the sample splice site from the subject;
(h) repeating steps (b-f) to identify unique variants in the CSP database that affect the same splice site as the sample splice site that are calculated to have a similar NIF-Shift as determined in (f);
(i) determining the clinical classification(s) associated with each unique variant in the CSP database affecting the same splice site and the sample splice site from the test subject that are determined to have a similar NIF-Shift determined in (f); and
(j) determining the risk of abnormal splicing or likelihood of maintaining normal splicing for the sample splice site in the test subject by assessing the clinical classification determined in step (k) for each unique variant in the CSP database that affect the same splice site and are determined to have a similar NIF-shift variant identified in step (f).

19-20. (canceled)

21. The method of claim 1, wherein the sample splice site sequence is a donor splice site sequence, a branch site sequence, or an acceptor splice site sequence.

22. (canceled)

23. The method of claim 1, wherein each sample splice site sequence comprises at least 4 to 15 consecutive nucleotides of a donor splice site.

24-28. (canceled)

29. The method of claim 1, wherein at least one sample splice site sequence corresponds to nucleotide positions E⁻⁴ to D⁺⁵, E⁻³ to D⁺⁶, E⁻² to D⁺⁷ and E⁻¹ to D⁺⁸ of a donor splice site.

30. (canceled)

31. The method of claim 1, wherein the sample splice site is obtained by sequencing the splice site of a predetermined gene.

32-45. (canceled)

46. A method of providing a risk of abnormal splicing of a sample splice site from a subject, said method comprising:
obtaining a first sample splice site sequence comprised in the sample splice site froth the subject;
generating a first abnormal splicing factor based on a measure of Native Intron Frequency (NIF) of the sample splice site (NIFᵥₐᵣ₋₁) and a measure of NIF of a first reference splice site (NIF_{ref-1});
generating a second abnormal splicing factor by comparing the sample splice site sequence to pre-classified data wherein the pre-classified data includes splice site sequences which have been classified as an abnormal splice site or a benign variant splice site;
generating a third abnormal splicing factor based on pre-classified splice site sequences having a similar NIF_{vaf-1} and a similar corresponding NIF_{ref-1}; and
generating a risk of abnormal splicing of the sample splice site by evaluating the first, second, and third abnormal splice factors.

47.-55. (canceled)

56. The method of claim 12, wherein at least one sample splice site sequence corresponds to nucleotide positions E⁻⁴ to D⁺⁵, E⁻³ to D⁺⁶, E⁻² to D⁺⁷ and E⁻¹ to D⁺⁸ of a donor splice site.
